SEQUENCE LISTING (98 sequences)

SEQ ID NO: 1 - DNA, 2163 bp, Cannabis sativa
  source 1..2163: mol_type = genomic DNA
atgggaaaaaattataagtcacttgacagtgtggttgctagtgatttcatagccttgggtatcacatctgaggtagcggagactcttcatgggcgtttagctgagattgtgtgtaattacggggcggccacccctcagacttggatcaatatagcaaaccacatcttatctcctgatctacccttttctctacaccaaatgctgttctatgggtgttacaaagatttcgggcccgcaccgcccgcatggataccggatccagagaaagtcaaatccaccaacttaggcgcgttgcttgaaaaaagaggcaaggaattcctgggcgtcaagtacaaggaccccatctcatctttctcccacttccaagagttctctgtacgtaatccggaggtttactggcgtaccgtccttatggatgagatgaagatatcattctccaaggacccggaatgtatactaagaagggatgacattaacaacccagggggcagtgagtggcttccgggtggataccttaatagtgcaaaaaactgtcttaacgtgaactcaaacaaaaagttaaacgatacaatgatcgtgtggcgtgatgagggcaatgatgaccttccgctaaataagttgacgcttgatcagctaagaaagcgtgtctggcttgtcggatatgccctagaggagatgggccttgagaagggatgcgcgatcgccatagatatgcctatgcatgtcgatgctgtggttatatatctggccatcgttctagcgggctatgttgttgtgagtattgccgatagcttcagtgcacccgaaatatcaacgagactgcgtttgtccaaagccaaagcaatattcacacaggatcatatcattagaggaaagaagcgtattccgctatattcaagggtggtggaggccaaaagcccgatggctatagtgataccctgttcaggcagcaacatcggagctgaattaagagatggagatatatcctgggactattttttagagagggcgaaagaatttaagaactgcgagttcaccgcaagggaacagccagtggatgcgtatacaaacatactattcagttcaggtactacgggagaaccaaaggctataccctggacgcaggcaaccccgctaaaagctgccgctgacgggtggtctcatttggatatcaggaagggtgatgttatcgtatggccaacaaatcttggatggatgatgggaccgtggcttgtgtacgctagcttgttaaatggagcttcaatcgctctatataatggttcccctcttgtcagcggttttgcgaagttcgtacaggatgccaaggtaactatgctgggagtcgttcctagcatcgtcaggagttggaaatcaactaactgtgtgtctggttacgactggtccactatcagatgttttagctccagtggtgaggcctccaacgtagatgagtacctttggcttatgggacgtgccaactataaaccggtaatagagatgtgtgggggtactgaaataggaggagcgttttcagctggcagttttcttcaggctcaaagcttgtctagtttctcttcacagtgtatgggctgtacgttatacattcttgataagaacggttatccaatgcccaaaaacaaaccaggtataggagaattggcgctgggtccagtcatgtttggcgctagtaaaactctactgaacggaaatcatcacgacgtttactttaaaggtatgcctactcttaatggagaagtacttaggcgtcatggcgatatctttgagttgacatccaatggctactatcacgcccatggcagggcggatgacaccatgaacatcggggggatcaaaatctccagtatagagatcgagagagtgtgtaacgaggtagacgaccgtgtattcgagacaacagccattggggttccacccctaggtggtggccccgaacaacttgttatcttttttgtgctgaaggactcaaatgacactaccattgatttaaatcaactacgtttgtcattcaatctgggattacaaaagaagttgaatcctttattcaaggtcacaagagtagtaccccttagttccctgccaagaactgcgacaaacaagataatgcgtagagtgctaaggcagcagtttagtcattttgagtaa

SEQ ID NO: 2 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 3 - DNA, 1158 bp, Cannabis sativa
  source 1..1158: mol_type = genomic DNA
atgaaccacttgagagcagaagggccggccagtgtactggctatagggacagccaaccccgaaaatatactgttgcaagatgagttcccagattattactttagagtgactaaatccgagcacatgacgcaacttaaggagaaattcaggaaaatatgcgacaaatctatgattagaaaaagaaattgtttcttaaatgaggagcatctaaagcagaatccccgtctggtagaacatgaaatgcaaactttggacgcgcgtcaagacatgctagttgtcgaagtgcctaaattaggcaaagacgcgtgtgcaaaggctataaaagagtggggccaaccgaagtccaaaattacacacctaatattcacttctgcgtccaccaccgacatgcccggagccgactaccactgtgcgaaacttctaggcctatccccttcagtcaagcgtgtaatgatgtatcaactggggtgctacggaggaggcaccgttttgaggattgcaaaggatatcgctgaaaataacaagggggctcgtgtacttgctgtgtgctgtgatatcatggcctgccttttcagaggcccctcagagtcagatcttgaactgttagtaggtcaggctatcttcggagatggcgctgcagccgtcatagttggggcggagcctgacgaatcagttggggagaggcccattttcgagctggtcagtacgggacagaccatcttgccaaatagcgagggcacgatcggaggccacataagggaggcgggtttgatatttgaccttcataaggatgtaccgatgttgatctccaataatattgagaagtgtcttattgaagcatttacccctattggtatttcagactggaacagtatcttctggattacgcatccgggaggtaaggcgattcttgataaagtcgaagaaaagctacacctgaagtcagacaagttcgttgactccagacacgttctttcagagcacggcaacatgagttcttccaccgtccttttcgtaatggacgagctgaggaaacgtagccttgaggaaggtaaaagtacgacaggagatgggtttgagtggggagtgttgtttggcttcggcccagggttaacagttgaacgtgtagtcgttagatctgtccctattaaatactaa

SEQ ID NO: 4 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 5 - DNA, 306 bp, Cannabis sativa
  source 1..306: mol_type = genomic DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaataa

SEQ ID NO: 6 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 7 - DNA, 1517 bp, Cannabis sativa
  source 1..1517: mol_type = genomic DNA
atcaataataatcttcatgggactctcattagtttgtaccttttcatttcaaactaattatcatactttattaaaccctcataataagaatcccaaaaactcattattatcttatcaacaccccaaaacaccaataattaaatcctcttatgataattttccctctaaatattgcttaaccaagaactttcatttacttggactcaattcacacaacagaataagctcacaatcaaggtccattagggcaggtagcgatcaaattgaaggttctcctcatcatgaatctgataattcaatagcaactaaaattttaaattttggacatacttgttggaaacttcaaagaccatatgtagtaaaagggatgatttcaatcgcttgtggtttgtttgggagagagttgttcaataacagacatttattcagttggggtttgatgtggaaggcattctttgctttggtgcctatattgtccttcaatttctttgcagcaatcatgaatcaaatttacgatgtggacatcgacaggataaacaagcctgatctaccactagtttcaggggaaatgtcaattgaaacagcttggattttgagcataattgtggcactaactgggttgatagtaactataaaattgaaatctgcaccactttttgttttcatttacatttttggtatatttgctgggtttgcctattctgttccaccaattagatggaagcaatatccttttaccaattttctaattaccatatcgagtcatgtgggcttagctttcacatcatattctgcaaccacatcagctcttggtttaccatttgtgtggaggcctgcttttagtttcatcatagcattcatgacagttatgggtatgactattgcttttgccaaagatatttcagatattgaaggcgacgccaaatatggggtatcaactgttgcaaccaaattaggtgctaggaacatgacatttgttgtttctggagttcttcttctaaactacttggtttctatatctattgggataatttggcctcaggttttcaagagtaacataatgatactttctcatgcaatcttagcattttgcttaatcttccagactcgtgagcttgctctagcaaattacgcctcggcgccaagcagacaattcttcgagtttatctggttgctatattatgctgaatactttgtatatgtatttatataagaccataatataacatatatatgtttattacataaaattgggacacaaaaacgtcaattatttggacaaaagtactcagaaagacctctttcactacaaggggaggccatttagttatacttgggtttcaatcaacaaatttataaatttttaagattttatttacaaaacattttcatgtgtaattaaatcgatcgtcatttattttttggatacaacttggttcaacttattttaattagagtgcttcgtaatttaactacaattatagaagggcattttataaaaatactggatttggggt

SEQ ID NO: 8 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 9 - DNA, 954 bp, synthetic construct
  misc_feature 1..954: note = Synthetic polynucleotide
  source 1..954: mol_type = other DNA
atgatcgaaggttcacctcatcatgaaagtgataacagcatagccacgaagattttgaatttcggccatacttgttggaagctacagaggccgtacgtcgttaaggggatgatttccattgcgtgcggtctgtttggcagggaattatttaacaacagacacttattcagttggggcctgatgtggaaggccttcttcgctcttgtacccattctgtccttcaacttttttgcagcgatcatgaatcaaatatacgatgtagacatcgatagaataaacaagcccgatttacctctggtatcaggcgaaatgagcatcgaaactgcgtggattttatcaatcatcgttgcattgactgggctgatagtgaccataaagttaaagtcagccccgttgtttgtcttcatatacatcttcggcattttcgcgggctttgcgtatagtgtacctcccattagatggaagcagtacccgtttactaactttcttattacaattagcagccatgtcggtcttgcattcacgtcctactcagccaccacatccgcactggggctaccgtttgtgtggcgtccagccttcagcttcatcatcgcattcatgacagtaatgggtatgacgatagcttttgcaaaggatataagtgatatcgagggtgacgctaagtatggagtgtctactgtggccacgaagctgggggcccgtaatatgactttcgtggtatcaggtgtactattgcttaattaccttgtttctatatcaatcggaattatttggccacaagttttcaaatccaatataatgatcctatcacacgctattttagcgttttgtttgatatttcagactagagagcttgcactagcgaattacgcgagtgccccgagtaggcagtttttcgagttcatatggctattatactatgctgagtactttgtttacgtatttatttaa

SEQ ID NO: 10 - protein, 317 aa, synthetic construct
  REGION 1..317: note = Synthetic polypeptide
MIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 11 - DNA, 1635 bp, Cannabis sativa
  source 1..1635: mol_type = genomic DNA
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 12 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 13 - DNA, 1554 bp, synthetic construct
  misc_feature 1..1554: note = Synthetic polynucleotide
  source 1..1554: mol_type = other DNA
atgaacccccgtgaaaattttttgaaatgtttctctcaatacatacccaacaatgcaaccaacttaaagctggtatatactcaaaacaaccccctatatatgtctgttctaaatagtactatccataacttacgtttcacctcagataccacccctaaaccgctggtcatcgtgactccgtctcatgtttcacacatacagggcacgatattgtgctcaaaaaaggtcgggttacagattcgtacccgttcaggaggtcatgatagtgagggaatgtcttacatctcccaggtcccttttgtaattgtcgaccttcgtaatatgagatccataaagatcgacgttcattcacagacggcgtgggtagaggctggtgcaaccctaggtgaagtctactactgggtcaacgaaaaaaacgagaacttatcattagctgcggggtattgccctacagtttgtgccggaggtcattttggaggtggaggctacgggccactgatgaggaactacggtctggcagcagacaacattatagatgcacacctagtgaacgtgcatggtaaagttttagatagaaagtccatgggagaagatttgttttgggcactacgtggaggaggggctgagtcattcgggattattgtagcgtggaagatccgtctggtcgcagtccctaaatctacgatgttttccgtgaaaaagattatggaaattcacgagctagtgaaacttgtcaataagtggcagaatatagcatacaaatatgacaaggatctattgttgatgacgcatttcatcacaagaaacattacggacaatcaaggtaagaataagacggctattcacacttacttcagctccgtttttctaggaggggtagattccctagttgacctgatgaataagagttttcccgagttgggtattaaaaaaactgattgtagacagctgtcttggatcgacacaatcatattctactctggtgtggtaaactatgacaccgataatttcaacaaagaaatcttactggatagatcagccggtcaaaacggcgcgtttaaaatcaagctggattacgtaaagaagcctatacccgaatccgtatttgtacagattctggaaaagttatacgaggaagacattggggcgggtatgtacgctctttacccttacggcgggatcatggatgagatttccgaaagtgctatcccgttccctcatcgtgctggcattctgtacgagttatggtatatttgcagttgggagaagcaggaggataatgaaaagcacctaaattggattcgtaatatttataatttcatgactccctatgttagtaagaaccccagactggcctaccttaattatagagacctggacatcgggataaatgatccgaagaacccaaataactatacgcaggccaggatttggggggaaaagtatttcggaaagaactttgacagactggtgaaagttaagaccctggtggatccaaataattttttcaggaacgagcagagtattcccccgcttccacgtcacaggcattaa

SEQ ID NO: 14 - protein, 517 aa, synthetic construct
  REGION 1..517: note = Synthetic polypeptide
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 15 - DNA, 2685 bp, synthetic construct
  misc_feature 1..2685: note = Synthetic polynucleotide
  source 1..2685: mol_type = other DNA
atgagaacgcaaaagatcgtaacggttttatgcctactgcttaatactgtcttgggcgcgttactgggtatagattatggacaagagttcaccaaggccgtgttggtagcacctggtgtgccatttgaagttatccttacaccagatagcaagagaaaggacaactcaatgatggctattaaggaaaatagcaagggtgagatcgaacgttactatggttctagtgcctcatcagtatgtatcagaaatcctgaaacctgcttaaatcaccttaagtctctaattggcgttagtatagacgacgtgtcaactattgattacaaaaaataccattcaggagctgaaatggttccttcaaagaacaacagaaacactgtggcgtttaagttggggtctagcgtctatccggtagaagagatcttagctatgagcttagacgatattaaatccagagctgaagaccacttaaaacatgctgttcctgggtcatactctgtcataagcgatgccgtcattactgttccgaccttcttcacgcaaagccaaagattggccttaaaagatgctgcggaaatcagcggtttaaaagtcgtcggtttagttgatgatgggatctctgttgccgtcaactatgcatccagcagacaattcaacggcgacaaacagtatcacatgatctacgatatgggtgccggctcattacaagcgacattggtaagcatctctagcagcgacgatgggggaatcgtcatagatgtcgaagccatcgcctacgataaatcattgggtggtcaactgtttactcagtcagtctatgatatattattacagaagttcttgtctgaacatccttctttttccgaatctgactttaacaaaaactccaagagcatgtcaaagttgtggcaagctgccgagaaagcgaaaacgatccttagtgcgaacacagacactagagtcagtgtggaatccctttacaatgacattgacttcagagccacgattgccagggatgaatttgaagactacaacgctgaacacgtccataggatcacagccccaataattgaagccttgtcacatcctttgaacgggaaccttacatctccatttcctttgaccagtttgtcatcagtcatcttgacaggtggtagcacgagagtcccgatggtcaagaaacatcttgaatcactacttgggtctgagctgatagcaaagaatgttaatgctgatgaatcagcagtatttggaagcactcttcgtggtgtcacactatctcaaatgtttaaagccaagcagatgacggttaacgagaggtccgtgtacgattattgtcttaaagttggttcatctgagatcaacgtttttcctgttggaactccattagccacaaagaaagtggttgaactggaaaatgtggacagcgaaaaccaactgaccataggtctgtacgagaatgggcaactatttgcctctcatgaagtaactgatcttaaaaaatccatcaagagtttgacacaagaggggaaagagtgctccaatataaattacgaagcgactgtcgaattaagtgaatcccgtttgttgtctttaacaagacttcaggcgaaatgcgcagacgaggcagaatatttgcctccagttgatacggaatccgaagataccaaatctgagaatagcacaacctcagaaactatagagaagcctaacaagaagttattctacccggttactataccgacccagttgaaatctgtgcatgtaaagcctatgggttcaagcaccaaagtttccagttctttaaagattaaggaactgaataaaaaagatgcggtgaaacgtagcatagaagagttgaagaaccagcttgagtcaaagctgtacagagtcaggtcctatttggaagatgaggaagtggtggagaaaggtccagcttcacaagtagaagcattgtctactttagtagccgaaaacttggaatggttggactatgatagtgatgatgcgtcagctaaggacataagggaaaagcttaattcagtgagcgacagtgtcgcgttcattaagtcatacatcgatttgaatgatgtcacgttcgataacaacctttttacaacaatctacaacaccaccttaaattccatgcaaaatgtgcaggagctaatgctaaacatgtctgaagatgccctttcattaatgcaacagtacgaaaaggaaggtcttgatttcgccaaggaatctcagaaaattaagattaagtcaccaccactatctgacaaagagttggataacctgttcaacacagtcaccgaaaagttggaacacgtaagaatgcttactgaaaaggacacgatctctgacctgcctagagaagaactgttcaagctgtatcaggaactgcaaaactactcctctagattcgaagccattatggccagtttagaggatgtccattcacagaggattaaccgtttaactgacaaactgagaaaacacatcgaaagggtgtctaatgaagcgctaaaagcagccttgaaagaggctaaaaggcagcaagaggaagagaaatctcatgaacagaacgaaggagaagaacaatcatctgcgagtacatcccataccaacgaagacatcgaagaaccaagtgaaagccctaaggtccaaaccagtcacgatgaattgtaa

SEQ ID NO: 16 - protein, 894 aa, Pichia pastoris
MRTQKIVTVLCLLLNTVLGALLGIDYGQEFTKAVLVAPGVPFEVILTPDSKRKDNSMMAIKENSKGEIERYYGSSASSVCIRNPETCLNHLKSLIGVSIDDVSTIDYKKYHSGAEMVPSKNNRNTVAFKLGSSVYPVEEILAMSLDDIKSRAEDHLKHAVPGSYSVISDAVITVPTFFTQSQRLALKDAAEISGLKVVGLVDDGISVAVNYASSRQFNGDKQYHMIYDMGAGSLQATLVSISSSDDGGIVIDVEAIAYDKSLGGQLFTQSVYDILLQKFLSEHPSFSESDFNKNSKSMSKLWQAAEKAKTILSANTDTRVSVESLYNDIDFRATIARDEFEDYNAEHVHRITAPIIEALSHPLNGNLTSPFPLTSLSSVILTGGSTRVPMVKKHLESLLGSELIAKNVNADESAVFGSTLRGVTLSQMFKAKQMTVNERSVYDYCLKVGSSEINVFPVGTPLATKKVVELENVDSENQLTIGLYENGQLFASHEVTDLKKSIKSLTQEGKECSNINYEATVELSESRLLSLTRLQAKCADEAEYLPPVDTESEDTKSENSTTSETIEKPNKKLFYPVTIPTQLKSVHVKPMGSSTKVSSSLKIKELNKKDAVKRSIEELKNQLESKLYRVRSYLEDEEVVEKGPASQVEALSTLVAENLEWLDYDSDDASAKDIREKLNSVSDSVAFIKSYIDLNDVTFDNNLFTTIYNTTLNSMQNVQELMLNMSEDALSLMQQYEKEGLDFAKESQKIKIKSPPLSDKELDNLFNTVTEKLEHVRMLTEKDTISDLPREELFKLYQELQNYSSRFEAIMASLEDVHSQRINRLTDKLRKHIERVSNEALKAALKEAKRQQEEEKSHEQNEGEEQSSASTSHTNEDIEEPSESPKVQTSHDEL

SEQ ID NO: 17 - DNA, 2130 bp, synthetic construct
  misc_feature 1..2130: note = Synthetic polynucleotide
  source 1..2130: mol_type = other DNA
atggctagtgaaacttttgaatttcaagctgaaattactcagttgatgagtttgatcatcaacaccgtctattctaacaaggaaattttcttgagagaactgatatctaatgcctcggatgcgttggataaaattagatacaaatctttgtctgatccaaagcaattggaaacagaaccagatctctttattagaatcactccaaagccagagcaaaaagttttggaaatcagagattctggtattggtatgaccaaggctgaattgattaataacttgggtaccattgccaagtctggtaccaaagccttcatggaagctctatctgctggtgccgatgtatccatgattggtcaattcggtgttggtttttactctttattcttagttgccgacagagttcaggttatttcaaagagcaacgacgacgaacaatacatctgggaatccaacgctggtggttctttcactgttactctagacgaagttaatgaaagaattggtaggggtaccatcttgaggttattcttgaaagatgaccaattggagtacttggaagaaaagagaataaaggaagttatcaagagacattctgagttcgtggcctacccaatccaattagtcgtcaccaaggaagttgaaaaggaagttccaattccagaagaagaaaagaaagacgaggaaaagaaggatgaggaaaagaaggatgaagacgacaagaaaccaaaattggaagaagtcgatgaagaagaggaaaagaagccaaagacgaaaaaagttaaagaagaagttcaagagatagaagaactaaacaagactaagcctttgtggactagaaacccatctgatatcactcaagaagaatacaatgctttctataagtctatttcaaacgactgggaagacccattgtacgttaagcatttctccgttgaaggtcaattggaatttagagctatcttattcattccaaagagagcaccattcgacttgtttgagagtaaaaagaagaagaataatatcaagttgtacgttcgtcgtgttttcatcactgatgaagctgaagacttgattccagagtggttatctttcgtcaagggtgttgttgactctgaggatttaccattgaatttgtccagagaaatgttacaacaaaataagatcatgaaggttattagaaagaacattgtcaaaaagttgattgaagccttcaacgaaattgctgaagactctgaacaatttgaaaagttctactcggctttctccaaaaatatcaagttgggtgtacatgaagatacccaaaacagggctgctttggctaagttgttacgttacaactctaccaagtccgtagatgagttgacttccttaactgattacgttaccagaatgccagaacaccaaaagaacatctactacatcactggtgaatctctaaaggctgtcgaaaagtctccatttttggatgccttgaaggctaaaaacttcgaggttttgttcttgaccgacccaattgatgaatacgccttcactcaattgaaggaattcgaaggtaaaactttggttgacattactaaagatttcgaattggaagaaactgacgaagaaaaagctgaaagagagaaggagatcaaagaatatgaaccattgaccaaggccttgaaagaaattttgggtgaccaagtggagaaagttgttgtttcttacaaattgttggatgccccagctgctatcagaactggtcaatttggttggtctgctaacatggaaagaatcatgaaggctcaagccttgagagactcttccatgtcctcctacatgtcttccaagaagactttcgaaatttctccaaaatctccaattatcaaggaattgaaaaagagagttgacgaaggtggtgctcaagacaagactgtcaaggacttgactaagttattatatgaaactgctttgttgacttccggcttcagtttggacgaaccaacttcctttgcatcaagaattaacagattgatctctttgggtttgaacattgatgaggatgaagaaacagagactgctccagaagcatccaccgcagctccggttgaagaggttccagctgacaccgaaatggaagaggtagattag

SEQ ID NO: 18 - protein, 709 aa, Saccharomyces cerevisiae [sequence omitted]

SEQ ID NO: 19 - DNA, 1587 bp, synthetic construct
  misc_feature 1..1587: note = Synthetic polynucleotide
  source 1..1587: mol_type = other DNA
atgtctgctaaagttccatctaacgccacgtttaagaacaaggaaaaacctcaagaggttcgcaaagccaacatcatcgctgcacgttctgttgcagatgccatccgtacttcattgggtcccaagggtatggacaagatgattaagacatctcgtggagaaatcatcatctctaatgatggccacaccattctaaaacagatggccattctgcatccggtggccagaatgctagtagaggtttctgccgcgcaggactcggaagccggtgatggtaccacttctgtggtgatcttgaccggagctctattgggtgctgctgagaggctgttaaacaagggcatccatccaaccatcattgcggactcctttcaaagtgctgcgaagagatctgtcgatattcttttagaaatgtgccataaggtttcgttgagcgatagagaacagctggttcgtgctgcatccacttctttgagttccaagattgtgtctcaatactcttcgtttttggcgcccttggcagtggactccgtattgaagatatctgatgaaaactctaagaatgttgacctgaacgatatcagactggtcaaaaaagttggtggtaccattgatgacacagaaatgatagatggtgtggtcttgacacaaacggcaatcaaatctgctggtggtccgacaagaaaggaaaaagcaaagattgggttaattcaattccaaatatctcctccaaagcccgacacagaaaataatatcatcgttaatgactatagacaaatggataagatccttaaagaagaaagagcgtatttgctaaatatctgtaaaaaaattaaaaaggccaagtgtaacgtgctgttgattcagaaatccatcttgagagatgcggtaaatgatttggctcttcatttcttgtcaaaattgaacataatggtggtaaaggatatcgagagagaagaaatcgagtttctgtcgaagggcttgggttgtaagccaattgctgatatagaattgttcaccgaagatagattgggttccgctgatttagttgaggagatcgatagcgatggttccaagatagttagagtaactggtataagaaacaataatgcaagaccaaccgtctctgttgttattcgtggtgcaaacaacatgatcattgatgaaacagagcgttccctccatgatgcactatgtgttattcgttgtctagtgaaagagagaggtttaatcgctggtggtggtgctccagaaattgaaatatcccgtaggttgagtaaggaagcaaggtctatggaaggtgtacaggcttttatttggcaagaatttgcctctgctttggaagttattccaacgactttggctgaaaacgcaggtttgaatagtatcaaagttgtcacagaattgcgctctaagcatgaaaatggtgaattgaatgacggtatctctgttagacgatctggtactacgaacacctatgaagaacacatcttacaacccgtacttgttagtacaagtgcaattaccttagcgtctgaatgtgttaaatccatcttacgtattgatgatattgcattcagccgttaa

SEQ ID NO: 20 - protein, 528 aa, Saccharomyces cerevisiae
MSAKVPSNATFKNKEKPQEVRKANIIAARSVADAIRTSLGPKGMDKMIKTSRGEIIISNDGHTILKQMAILHPVARMLVEVSAAQDSEAGDGTTSVVILTGALLGAAERLLNKGIHPTIIADSFQSAAKRSVDILLEMCHKVSLSDREQLVRAASTSLSSKIVSQYSSFLAPLAVDSVLKISDENSKNVDLNDIRLVKKVGGTIDDTEMIDGVVLTQTAIKSAGGPTRKEKAKIGLIQFQISPPKPDTENNIIVNDYRQMDKILKEERAYLLNICKKIKKAKCNVLLIQKSILRDAVNDLALHFLSKLNIMVVKDIEREEIEFLSKGLGCKPIADIELFTEDRLGSADLVEEIDSDGSKIVRVTGIRNNNARPTVSVVIRGANNMIIDETERSLHDALCVIRCLVKERGLIAGGGAPEIEISRRLSKEARSMEGVQAFIWQEFASALEVIPTTLAENAGLNSIKVVTELRSKHENGELNDGISVRRSGTTNTYEEHILQPVLVSTSAITLASECVKSILRIDDIAFSR

SEQ ID NO: 21 - DNA, 1689 bp, synthetic construct
  misc_feature 1..1689: note = Synthetic polynucleotide
  source 1..1689: mol_type = other DNA
atggctgctcgtccacaacaacctcctatggaaatgccggatctgtcgaacgccattgtggcacaagacgagatgggtagaccctttattatcgtgaaggatcaaggtaacaagaagagacagcacggattagaggccaagaaatcacatatattggctgctagatcagtagcttctattatcaaaacttcgttgggtccccgtgggttagataagatcttgatttcacctgacggtgaaatcaccatcactaacgatggtgctacaattttgtcccaaatggagctagataatgagattgcaaagttgttagtacaactttccaaatctcaagatgatgaaattggtgatggtactactggtgttgttgttctggcaagtgcgctacttgaccaagcattggagttaattcaaaagggtatacatccaatcaaaatcgctaatggatttgatgaagccgccaaattagccatttccaagttagaagaaacatgtgacgacatttctgcgtcaaacgatgaattgttcagggactttttattgagggccgccaaaacttctttgggctccaaaattgtttccaaggatcatgatagatttgctgaaatggctgtggaagccgtcataaatgtcatggacaaagatcgtaaagacgtggatttcgatttaatcaaaatgcaaggacgggttggcggatctataagcgattcaaaattgattaatggtgtcattttggataaagatttttctcatccacaaatgcctaaatgtgttttaccaaaagagggctctgatggtgttaagttagctatattaacgtgtccatttgaacctcctaaaccaaagaccaagcataaattagacatttcttcagtagaagaataccaaaaattacagacttatgaacaagataagttcaaagaaatgatagacgatgtgaagaaagcgggcgcagatgttgttatatgccaatgggggtttgacgatgaggccaatcatctacttctacagaatgatttacctgccgtaagatgggtaggtggccaagaactagaacacattgccatttccacaaacggtcgcattgttccaagatttcaagacttgtctaaggataaattaggtacatgttccagaatttacgagcaggagtttggtactactaaggatcgtatgctgattatcgagcaaagtaaagaaacaaaaactgtaacatgttttgttcgcggttccaataaaatgatcgtggatgaagctgaacgtgcattgcatgactcactatgtgtggtacgtaacttggttaaagactcacgtgtagtttacggtgggggagcagccgaagtgactatgtccctggctgtctctgaggaagctgataagcaacgtggtattgatcagtatgcattccgtggattcgcccaagcgttagacaccattccaatgactttggctgaaaattctggtcttgatccaatcggaactttgtctacattgaaaagtaaacaactgaaagaaaagatttccaacattggtgtcgattgcttaggttacgggtctaatgacatgaaggaactatttgtcgttgatccatttattggcaaaaagcagcaaattcttttagctactcaattatgtagaatgattttaaagattgataacgtcatcattagtggtaaagatgagtattga

SEQ ID NO: 22 - protein, 562 aa, Saccharomyces cerevisiae
MAARPQQPPMEMPDLSNAIVAQDEMGRPFIIVKDQGNKKRQHGLEAKKSHILAARSVASIIKTSLGPRGLDKILISPDGEITITNDGATILSQMELDNEIAKLLVQLSKSQDDEIGDGTTGVVVLASALLDQALELIQKGIHPIKIANGFDEAAKLAISKLEETCDDISASNDELFRDFLLRAAKTSLGSKIVSKDHDRFAEMAVEAVINVMDKDRKDVDFDLIKMQGRVGGSISDSKLINGVILDKDFSHPQMPKCVLPKEGSDGVKLAILTCPFEPPKPKTKHKLDISSVEEYQKLQTYEQDKFKEMIDDVKKAGADVVICQWGFDDEANHLLLQNDLPAVRWVGGQELEHIAISTNGRIVPRFQDLSKDKLGTCSRIYEQEFGTTKDRMLIIEQSKETKTVTCFVRGSNKMIVDEAERALHDSLCVVRNLVKDSRVVYGGGAAEVTMSLAVSEEADKQRGIDQYAFRGFAQALDTIPMTLAENSGLDPIGTLSTLKSKQLKEKISNIGVDCLGYGSNDMKELFVVDPFIGKKQQILLATQLCRMILKIDNVIISGKDEY

SEQ ID NO: 23 - DNA, 318 bp, synthetic construct
  misc_feature 1..318: note = Synthetic polynucleotide
  source 1..318: mol_type = other DNA
atgttgataggatacatcttcttgaccattgcgatttgctctgagagtattggtgctgctatgttgaaggtttcagatggtttcaagaagtggaaaccatctgccttagtcgtgattggatactccttggcgttctatatgctgtctcttactttgaaccacattcctttgtcattatcctacgctacatggtcaggtgctggtactgttcttactacagtaatcggggtgaaatggttcaaagaggatctaaatgccaaagggttaattggcattcttctactgttgtcaggtgttgtccttttgaactggccgtaa

SEQ ID NO: 24 - protein, 105 aa, Bacillus subtilis
MLIGYIFLTIAICSESIGAAMLKVSDGFKKWKPSALVVIGYSLAFYMLSLTLNHIPLSLSYATWSGAGTVLTTVIGVKWFKEDLNAKGLIGILLLLSGVVLLNWP

SEQ ID NO: 25 - DNA, 993 bp, synthetic construct
  misc_feature 1..993: note = Synthetic polynucleotide
  source 1..993: mol_type = other DNA
atgaatacacaaccaacaagggcgatagaaacctcaggcttggttaaggtctataacggtactagagctgtcgatggtctggatcttaacgtacctgcgggtctggtctatgggattttgggtccaaatggtgctggtaaatcaaccaccatcagaatgcttgctactcttctgcgtcctgacggtggcactgctagagttttcggtcatgatgtgacatctgaaccagacacagttagaaggagaatttctgtgacaggacaatacgcatccgtagacgaaggtttaactggaaccgaaaacctggtcatgatgggaaggttacaaggctattcatgggctagggccagagaaagagctgcggaattgatcgatggtttcggtcttggtgatgctagggatagacttcttaagacctattcaggaggaatgagaagaaggttggatattgcggcgtcaattgtggttactccagatttgctgttccttgatgaaccgactactggtttagatccaaggtcaaggaaccaggtttgggacattgtgagggcattagttgatgccggcactactgtgctattgacaacccagtacctggatgaagctgaccaacttgccgatagaattgcagtcatcgatcatggtagggtaatagctgaaggtactactggggaactgaagtcctcattgggctcaaacgtattgagattgaggctgcatgatgcccagtcaagagctgaagctgaaagattgctatcagccgaattgggcgtaactattcacagggacagtgatcctactgccttatctgcaaggatcgatgatcccagacaaggaatgagagctcttgctgaactgagcagaacgcatttagaggtgaggagcttttcactgggacaatcaagcttggacgaggttttcttggctttgacggggcatccagcagatgacagatctactgaagaagctgctgaagaggaaaaggtcgcataa

SEQ ID NO: 26 - protein, 330 aa, Streptomyces peucetius
MNTQPTRAIETSGLVKVYNGTRAVDGLDLNVPAGLVYGILGPNGAGKSTTIRMLATLLRPDGGTARVFGHDVTSEPDTVRRRISVTGQYASVDEGLTGTENLVMMGRLQGYSWARARERAAELIDGFGLGDARDRLLKTYSGGMRRRLDIAASIVVTPDLLFLDEPTTGLDPRSRNQVWDIVRALVDAGTTVLLTTQYLDEADQLADRIAVIDHGRVIAEGTTGELKSSLGSNVLRLRLHDAQSRAEAERLLSAELGVTIHRDSDPTALSARIDDPRQGMRALAELSRTHLEVRSFSLGQSSLDEVFLALTGHPADDRSTEEAAEEEKVA

SEQ ID NO: 27 - DNA, 1188 bp, synthetic construct
  misc_feature 1..1188: note = Synthetic polynucleotide
  source 1..1188: mol_type = other DNA
atgaactctaacaacagcaacacagagtttgatggtgttagggcacgtcagagcagaaaaaagggtttccttgggttggcagccgccatcgtcgtagctggtggttcatacgccctttattggcatttcataggaagtcgttacatatccacagataacgcatacgcagccgctgagattgccgaagttacgcctgctgtcggcagagattaccgtgcatctgaatgtcgttggattccatcaacatctacgggtgatgtgctagtgcaacttgacgataccgatgcaaggctggcattactgcaagctgaagctgatttagcacttgccaagcgtcgtgtcaggtcctatctggcgaacgacgaaggactgtctgcaatggttgaagctcaagaagccaatgaacaacgtgtgaaggctcagcttaaagctgctcaagccgattttgagagagctaaaatcgacttatctaggagagaggatcttgtcgcttcagggtctgtttctggcgaagaacttactaatgctaagacaggattcgctcaagcacaagcgaaccttaatgcagctaaagcagctatggcacaagctcaagctaccaaactgtcaactatcggtagccaaaaggccaatgctgcccttacagacaatactaccgtcgatagcaatccagaggttttgttggcaaaagctaggtacgaacaagccaagatagaccttgagagaacagttattagagctcctatcagcggtatagttgcgaaaagacaagttcaagtcggaagaagggtacaggttggaatgcctcttatgacagtcgttcctacggatcacatctacgttgacgctaacttcaaggaagtagaattgagggacgttaaggtgggtcagcctgtcactttgactgcagatttgtacggtgatgacgtaacatatcatggtgttgtagccggtttctctggaggaactggaagcgccttcagtatgatacctgcacaaaatgcaactggtaattggatcaaagtagtccagagattgcccattagaatagagttggacccaaaagatttgcaagcataccctttacaagtgggtctgtctatggtggctaccattgatacagccggtaccactgatcctcaaacacttgtccaatatagagcagccaaagtttctgaacagggttaa

SEQ ID NO: 28 - protein, 395 aa, Vibrio cholerae
MNSNNSNTEFDGVRARQSRKKGFLGLAAAIVVAGGSYALYWHFIGSRYISTDNAYAAAEIAEVTPAVGRDYRASECRWIPSTSTGDVLVQLDDTDARLALLQAEADLALAKRRVRSYLANDEGLSAMVEAQEANEQRVKAQLKAAQADFERAKIDLSRREDLVASGSVSGEELTNAKTGFAQAQANLNAAKAAMAQAQATKLSTIGSQKANAALTDNTTVDSNPEVLLAKARYEQAKIDLERTVIRAPISGIVAKRQVQVGRRVQVGMPLMTVVPTDHIYVDANFKEVELRDVKVGQPVTLTADLYGDDVTYHGVVAGFSGGTGSAFSMIPAQNATGNWIKVVQRLPIRIELDPKDLQAYPLQVGLSMVATIDTAGTTDPQTLVQYRAAKVSEQG

SEQ ID NO: 29 - DNA, 1188 bp, synthetic construct
  misc_feature 1..1188: note = Synthetic polynucleotide
  source 1..1188: mol_type = other DNA
atggatagcgatagagatgctgctgctttgaggaggcaattagctgatcctcttcttgctgctactcaccctgtgtacagaccattgttatggacccttctaggttgcgttctgttgttcattggttgggccgcttgggcacaactagatgaagtgacacgtggagatgggagagttgtgccattttccaggatacaaaagattcagagtttagaaggaggtatccttgatagactgttagtcaaggaaggcgatcttgttgaggttggacaaccactagttagactagacgaaactcgttttttgactaacttccaagaatcagcgaaccaggcttctgtgttaagggcagcaattgctagacttgatgcagaggtcttaggcaagaaatccattgagttcccaccagatgttgacccagaaggtcctctagcaagaagcgaaagggaactgttcaaatccagaagggacaaactggtggaaggtactcaggccattcagagacagattcatctggctcagtcacagctggacttagttagaccactagtcgctaaaagagctgtttcacagatggaggcccttaagctaagccaggacatcgcaaccctatctggcaaactaacggaactgaagtctacctactttcaagatgcctacacagaaagagcgcaaaggaaagctgatttgtctgctttagagcctatcgtacagcaaagacaagaccaactgagaagaacggagattttgtctccagtgagagggcgtgtcaacaccgttttgatcaacactagaggaggtgtcatccaacctggagaacctatcatggaagtgataccggttgaagaacgtcttcttgtcgaagccaagatcaagccaagagacgttgccttcttagtgcctggtatgccagcaaaagtcaagataactgcatacgactacacgatctatggggatctgaaaggaacgttggaacaaatctcagcggataccattgaagaagatacacctcatgggaaagaaagctactatcaagttctaatcaagacggatggtagccagttgaagagaggcgaggaggtcctaccaatcattccaggtatggtagcagaggttgacatactaagcggtaagagatctgttttgaactacttgctacgtcctcttatcaaggctagactatactaa

SEQ ID NO: 30 - protein, 395 aa, Pseudomonas aeruginosa
MDSDRDAAALRRQLADPLLAATHPVYRPLLWTLLGCVLLFIGWAAWAQLDEVTRGDGRVVPFSRIQKIQSLEGGILDRLLVKEGDLVEVGQPLVRLDETRFLTNFQESANQASVLRAAIARLDAEVLGKKSIEFPPDVDPEGPLARSERELFKSRRDKLVEGTQAIQRQIHLAQSQLDLVRPLVAKRAVSQMEALKLSQDIATLSGKLTELKSTYFQDAYTERAQRKADLSALEPIVQQRQDQLRRTEILSPVRGRVNTVLINTRGGVIQPGEPIMEVIPVEERLLVEAKIKPRDVAFLVPGMPAKVKITAYDYTIYGDLKGTLEQISADTIEEDTPHGKESYYQVLIKTDGSQLKRGEEVLPIIPGMVAEVDILSGKRSVLNYLLRPLIKARLY

SEQ ID NO: 31 - DNA, 1380 bp, synthetic construct
  misc_feature 1..1380: note = Synthetic polynucleotide
  source 1..1380: mol_type = other DNA
atgagtacatttcacaaggtcctgatcaatactatgatcagcaatgtcacaactggtttccttttctttgccgttgtcttttggatgtacctttccactggaaatgtggcgctaactggtattgtctctggcatctacatgggtttgattgcagtctgtagcattttcttcggtactgtagtagatcacaatcgtaaaaagtctgtcatgctgttttcctcagttacgaccctagtcttctactgtttgtctgccttagtttgggtgttttggctagaggaagacgggctatccattggtaatacagctttgtgggtttttgtgtcattcatactgattgggtcaatcgtcgagcatatgaggaacattgccctttctactgtcgtcactctgttagttccagaagcagaaagagataaggcgaatggtttagtaggtgccgttcagggagttgggttcttggtaaccagcgtcattgcaggttctgctattggcttcttaggcatggaaattacgctgtggatctgcttgggccttagcctagtcgctttgttgcacttattgcctataagagtggatgaacccgaaataattactcaggaagacgctcaacctactgtgtctgatgattcagttccaacaccgactagcgatctagctattgtatccaaaggcatcgatctgaaaggctccatgaagatcatactgtcagttccaggactactggcattggttctatttgcttcattcaacaacttaataggtggagtgtacagtgcgttgatggacccgtatggtttggaattattctcacctcagttatggggacttcttctgggtttaacgagtttgggtttcattgttggaggagcggttatctcaaagacggggcttggtaagaatccagtgaggactttgttgttagttaacgtaggtgttgcatttgtcggcatgttgttcgccatcagagaatggtggtggttgtacattttgggaatcttcattttcatggcaatcacgccagcagccgaagctgctgaacaaacgatcttgcagcgtgttgtaccatttagacaacagggaagagtctttggtttggctatggctgtggaaatggcagctaatccactttctacagtcatagttgcgattttagctgaggcatacctgatcccatggatggctggcccgggtgcagacactatctggggtgtgatccttggtgaaggcaaagctagaggaatggccctgatgttcttggcaagcggtgccatcatgttggtggttgtcttattagcatttatgtcaagatcctacaggaaactgtctcaatactacgctacaacaagccaggatattgcaggtgctgccgagaaataa

SEQ ID NO: 32 - protein, 459 aa, Corynebacterium glutamicum
MSTFHKVLINTMISNVTTGFLFFAVVFWMYLSTGNVALTGIVSGIYMGLIAVCSIFFGTVVDHNRKKSVMLFSSVTTLVFYCLSALVWVFWLEEDGLSIGNTALWVFVSFILIGSIVEHMRNIALSTVVTLLVPEAERDKANGLVGAVQGVGFLVTSVIAGSAIGFLGMEITLWICLGLSLVALLHLLPIRVDEPEIITQEDAQPTVSDDSVPTPTSDLAIVSKGIDLKGSMKIILSVPGLLALVLFASFNNLIGGVYSALMDPYGLELFSPQLWGLLLGLTSLGFIVGGAVISKTGLGKNPVRTLLLVNVGVAFVGMLFAIREWWWLYILGIFIFMAITPAAEAAEQTILQRVVPFRQQGRVFGLAMAVEMAANPLSTVIVAILAEAYLIPWMAGPGADTIWGVILGEGKARGMALMFLASGAIMLVVVLLAFMSRSYRKLSQYYATTSQDIAGAAEK

SEQ ID NO: 33 - DNA, 1380 bp, synthetic construct
  misc_feature 1..1380: note = Synthetic polynucleotide
  source 1..1380: mol_type = other DNA
atgttattagatttgaacaggttcagcttctctgtcttcttaaaggaggtgagattgctaactgcacttgccttgcccatgttgttggctcaagtggcccaggttggtattggctttgtcgataccgtaatggcaggaggtgctggtaaagaggacctggctgcagtagcactgggcagtagcgcttttgcgactgtctacatcacgtttatgggcataatggccgcccttaatcctatgatagcccagttatatggagctgggaaaacagacgaagttggagagactggtaggcaaggaatctggtttggactgtttctgggtgtattcggtatggtacttatgtgggccgctattacacccttcagaaactggttgacattgtcagattatgtcgaaggcactatggcccaatatatgctattcacttctcttgctatgccagcagctatggttcaccgtgcgttacacgcttacgcgtcctccttaaacagaccaaggttaataatgttggtctcttttgctgctttcgtcctgaacgtaccactaaactacatctttgtttatggcaagttcggtatgcccgctttaggaggcgctgggtgtggtttagccacaatggctgtcttctggttttcagcactagctttatggatctacattgcaaaggaaaatttcttcaggccatttggcctaacagcgaaattcggaaagcctgactgggcggttttcaaacaaatctggaagattggtgctccaataggtcttagctatttccttgaagcatcagcattctccttcatcgttttcctgatcgctccttttggtgaggactacgtggctgctcaacaggttggtatcagcttaagtggtattctgtacatgatccctcaatcagtcggatctgcaggtacggtcagaatcggcttctctttgggaagaagagagttcagtagagcgagatacatcagtggggtttcattggtctctggttggatgttggctgttatcacagtcttgtcacttgtgctattcagaagcccattggtttcaatgtacaacaacgatccagcagtcttgtctattgccgccactgtgttgttgtttgctgggctttttcaaccggcagacttcacccaatgtatcgcttcatacgctctaagaggatacaaggttacaaaggttcctatgttcatccatgctgcagctttttgggggtgtggattgttaccaggttacctacttgcatacaggttcgatatgggcatatacggattctggactgctcttatagcgtcactgacgattgctgctatcgctttagtttggtgcttggaattgtgcagcagagagatggtcagatctcacaaggctgtctaa

SEQ ID NO: 34 - protein, 459 aa, Neisseria sp.
MLLDLNRFSFSVFLKEVRLLTALALPMLLAQVAQVGIGFVDTVMAGGAGKEDLAAVALGSSAFATVYITFMGIMAALNPMIAQLYGAGKTDEVGETGRQGIWFGLFLGVFGMVLMWAAITPFRNWLTLSDYVEGTMAQYMLFTSLAMPAAMVHRALHAYASSLNRPRLIMLVSFAAFVLNVPLNYIFVYGKFGMPALGGAGCGLATMAVFWFSALALWIYIAKENFFRPFGLTAKFGKPDWAVFKQIWKIGAPIGLSYFLEASAFSFIVFLIAPFGEDYVAAQQVGISLSGILYMIPQSVGSAGTVRIGFSLGRREFSRARYISGVSLVSGWMLAVITVLSLVLFRSPLVSMYNNDPAVLSIAATVLLFAGLFQPADFTQCIASYALRGYKVTKVPMFIHAAAFWGCGLLPGYLLAYRFDMGIYGFWTALIASLTIAAIALVWCLELCSREMVRSHKAV

SEQ ID NO: 35 - DNA, 1554 bp, synthetic construct
  misc_feature 1..1554: note = Synthetic polynucleotide
  source 1..1554: mol_type = other DNA
atgtcacatgatccaactgctggaagagcagcagtagttgctcctcctaacaggcgtttatttgccgtaatgactgccgcggtcgtcgctatgctgttggctgctttggattccttaatcctgggaacggcgatgcctaccatcgtaggagaattgggtggactagaacacctgtcatgggttgcgtcagcttacatgttggcaacagctgctacaactcctatctgggggaaacttggggatatgtacggtaggaaaggcgtcttccttacgtccattgccctgttcctggctggatcagttgttgctggtctagctcaatccatgggtcagttgattgggtgtagagttttgcaagggctgggttcaggtgggttgatggtcggtgctcttagcttgatcggggaactagtgacacctagagaaagggggaagtttcaggccatgagttcagcagtgatgggtgtagcaatggtcggcggacctcttgctggagggtttctgacagatcacttaggttggcgttactgcttttacgtgaatgtcccattcggtctggccgcgtttgttacgatagcggtcgcgttaaaactgcctaagaggagatcacaagcacctattgattatcttggagctgctttgttggctgctgtaatcactagcctggtcttggttactacttggggtggaagacaatacgactggtcaagtccggtcattatcgggctgatagcaatagcggttgctggcgctttcacgttgcctgctgtcgagaggagagcagtcgctcctgtgcttcccctgactgtatttagatcagcaaacttttcctgggcaaacgttgtttctttcctgttcggttttgtgatggttgctgctatgactttcttaccgatttatcagcaaaccgtgcaaggagcttccgcttcatctgcgggactattgatgttgcctttattattaggtatggttggaaccaatgcttttggtggccaattgatgtcaaagcaagggaactttaagctgtatgcgattgcaggaggtgttttgctactggccggtattttactgttgtccctgatggatacaaaaacagggaggttagaaacaggtacgtatatggttgttttcggtgttggattaggtctgctgatgcaggttacattgagcgtggcgatggaatctgtggaaatgaaggatttaggagtggcttcctctatggtaaccttgacaaggaccataggcggagcttttggcgtcagtttagcaggtatcattttcaatgagagggttacggctggaatgggcgccgctggtggggctatgggtggagatatcaacaggcttagtgccgagagtcttcttaagctgccagaaccagttagagaagtttacgaaaaggccgtagctgatggtacacattgggctttccttactgttagcgtcgttgccgctgccgctcttgcagcgtcttggttcatcaaagctaggccaaagagcgccgcaccacccctggccacggcaccaactgaccaagtcggagctgccggttaa

SEQ ID NO: 36 - protein, 517 aa, Streptomyces cyanogenus
MSHDPTAGRAAVVAPPNRRLFAVMTAAVVAMLLAALDSLILGTAMPTIVGELGGLEHLSWVASAYMLATAATTPIWGKLGDMYGRKGVFLTSIALFLAGSVVAGLAQSMGQLIGCRVLQGLGSGGLMVGALSLIGELVTPRERGKFQAMSSAVMGVAMVGGPLAGGFLTDHLGWRYCFYVNVPFGLAAFVTIAVALKLPKRRSQAPIDYLGAALLAAVITSLVLVTTWGGRQYDWSSPVIIGLIAIAVAGAFTLPAVERRAVAPVLPLTVFRSANFSWANVVSFLFGFVMVAAMTFLPIYQQTVQGASASSAGLLMLPLLLGMVGTNAFGGQLMSKQGNFKLYAIAGGVLLLAGILLLSLMDTKTGRLETGTYMVVFGVGLGLLMQVTLSVAMESVEMKDLGVASSMVTLTRTIGGAFGVSLAGIIFNERVTAGMGAAGGAMGGDINRLSAESLLKLPEPVREVYEKAVADGTHWAFLTVSVVAAAALAASWFIKARPKSAAPPLATAPTDQVGAAG

SEQ ID NO: 37 - DNA, 1749 bp, synthetic construct
  misc_feature 1..1749: note = Synthetic polynucleotide
  source 1..1749: mol_type = other DNA
atgcacaatgacaaggatctatctacatggcaaacatttcgtagactatggcctaccattgctcctttcaaagccggtcttattgtcgctggtgtagctctgatcttgaatgctgcttctgacacgttcatgttatccctgttaaagcctctgctggatgatggattcgggaagacggacaggtccgtactagtctggatgccgctggtcgtaatcggactaatgattctaaggggtataacaagctacgtatcctcatactgcatatcatgggtatctggtaaagtggttatgactatgagaagaaggttgtttgggcacatgatgggtatgcctgtctctttctttgacaagcaatctactggcaccttactgtctaggattacctatgattcagaacaagtagcgagttccagttctggtgccttgatcactgttgttcgtgaaggcgcatctatcatcggtctgttcattatgatgttctactactcttggcaattaagcataatcctgatagtcttggctcctattgttagcatcgccataagagttgtctcaaagaggtttagaaacatatccaagaatatgcaaaacaccatgggtcaggttacgacctcagcggaacagatgctgaaggggcacaaagaagtcctgatatttggtggtcaggaagttgaaaccaaaagatttgataaggtgtctaacagaatgagactacaaggtatgaagatggtctcagcttcctctatctcagatcctattattcaactgatagcttctctagcgttggcttttgtcctttatgccgcaagtttcccgagtgtgatggattctctaactgctggtacgattactgtggtcttctctagcatgatagcccttatgagacccctgaagtcactgacaaacgtcaatgctcaattccaaaggggtatggcagcatgtcaaactctattcaccatattagattccgaacaggaaaaagacgaaggtaagagggtaatcgagcgtgcgactggagatgttgagtttagaaacgtcacctttacttaccctggtagagatgttcctgctctgcgtaacatcaatctgaaaatccctgctggaaaaactgttgccttggtaggtagaagcggttctgggaaaagcaccattgcatctttgatcaccaggttctacgatattgacgaaggcgaaatcctgatggacggacatgacctgagagagtatacgttggctagtctaagaaaccaagttgccttggttagccaaaacgtgcacctttttaatgacactgtggccaacaatatcgcatatgcgaggactgagcagtactcaagagaacaaatcgaagaagctgccagaatggcttatgccatggactttatcaacaagatggataacgggttggataccgttatcggagaaaacggcgtcttgttgtcaggtggtcagagacaaagaatcgccatagcgagagccttattgagggactcaccaatcttaattctggacgaagctacgtccgcacttgacacggaatcagaacgtgctattcaagctgcattggatgagttacaaaaaaacagaacgagcttagttatcgctcatcgtttaagcactatcgaaaaggctgacgaaatcgttgtagtggaagatggagtgattgtggaaagagggacgcataacgatttgcttgaacacagaggagtttacgcgcaattgcataaaatgcagttcggacaataa

SEQ ID NO: 38 - protein, 582 aa, Escherichia coli
MHNDKDLSTWQTFRRLWPTIAPFKAGLIVAGVALILNAASDTFMLSLLKPLLDDGFGKTDRSVLVWMPLVVIGLMILRGITSYVSSYCISWVSGKVVMTMRRRLFGHMMGMPVSFFDKQSTGTLLSRITYDSEQVASSSSGALITVVREGASIIGLFIMMFYYSWQLSIILIVLAPIVSIAIRVVSKRFRNISKNMQNTMGQVTTSAEQMLKGHKEVLIFGGQEVETKRFDKVSNRMRLQGMKMVSASSISDPIIQLIASLALAFVLYAASFPSVMDSLTAGTITVVFSSMIALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 39 - DNA, 1911 bp, synthetic construct
  misc_feature 1..1911: note = Synthetic polynucleotide
  source 1..1911: mol_type = other DNA
atgtatgttgatcctggtatcaacacttcagcgtccaacctaaagtgggtcctgccttactgtaaaccggatttaccgagagtcgttggtgctttgcttctgttcatcgctaacaataccatggccttaaccatcccaatactgtcaggcatcatagttgatagggttatcgtgggaggacatgctgacgagttacctcgtctttgcggtttaatgatattgatgacgataatcagagttggtgctcgttatggataccaaatgtggatggagagattcggtcaaaactccgtctataggttagtttccgatgagtatgaaaaactgcatgagctggactttacctactttaaccacacaagaacaggcgatatcatgtccagactgactagcgacaccgacgctataagacactgcttgtcttgggtttcctatcagattgccgactgtgtagtgatgttcgtgggtgcattgtgtgttatgttcgccatagactggagattagctctggtgttagcctgtgtcactccgttcattttcgtgctgacaagagggttatcaacgcatgctcatccactattttttgcaataagaaactccttagcatctttaaattctatggtcgaggagaacattgaaggaaatagggttgttaaagcctttgtaagggaaccctacgaaacggagaagtttgacgaacacaacgacgattacatgcaatgcaacatggctttagcctacaattccagaaagtacatgccttggctggacggtcttggattctcattacaattaataacactgggtttagggggctttttggtaattaaggggtacatgacccttggcaacctggtatctttcaattcttttctttggatgatcgatgggcctgtaagacagtcaggttggttaatcaatgactggcagaggttcaatgcaagctgtatcaaaattagaaagttgttgactgagcaaccaaggattgtagagaagccagatgctgttgaggctgtgaagcaagccgttactatcgtagagcaagtcaagcacgacgatgaagctaccggtgctacaggagcgcctgtggcgtcaggaacctcaggtgctcaacaatctacagaacgtaaaagctgtcctctgcatatcaaaggcgatatcagatttgaccacgtaagctttgcttttccggatgatccagaaacaccgattttgaaggatttggactttactgtacctgcaggttccaagttggggatcttaggtgagactggagccggcaagagcactctagtctccttgattagcaggttctatgatcctactgtaggacatgtgctgatcgatggcatagatgctcgtgattggccattggcaaccttaagatcacaggtctgtatagtcgcacaggatactttccttttctccgataccattggtggcaacatcggttttggtgcaagtggtgattcagatgacaggtatattcagaagatggctcaaatcgccggtgccgacaacttcatcaggtcaatgccacaaggttacgacactgtcgttggagaaagaggtgttggcttgtctggaggtcagaaacaaaggttgtcacttgctagggcattagctgacaatccgtccatattaataatggacgataccacttcagcagtcgacatggaaactgaagcagagattcagaaacatttgaaggagatggatggtggcaagactatcgtagcgatagctcacagaatctcctccgtgaaggattccgatttgatccttgtcttggaacatgggagaatcgtagaaaggggtacacatgctgaattggtggcagcacatggcagatactgggatatctaccacaaacagctgggtctgcagagcggagttgcgcaaggtttttaa

SEQ ID NO: 40 - protein, 636 aa, Bifidobacterium longum
MYVDPGINTSASNLKWVLPYCKPDLPRVVGALLLFIANNTMALTIPILSGIIVDRVIVGGHADELPRLCGLMILMTIIRVGARYGYQMWMERFGQNSVYRLVSDEYEKLHELDFTYFNHTRTGDIMSRLTSDTDAIRHCLSWVSYQIADCVVMFVGALCVMFAIDWRLALVLACVTPFIFVLTRGLSTHAHPLFFAIRNSLASLNSMVEENIEGNRVVKAFVREPYETEKFDEHNDDYMQCNMALAYNSRKYMPWLDGLGFSLQLITLGLGGFLVIKGYMTLGNLVSFNSFLWMIDGPVRQSGWLINDWQRFNASCIKIRKLLTEQPRIVEKPDAVEAVKQAVTIVEQVKHDDEATGATGAPVASGTSGAQQSTERKSCPLHIKGDIRFDHVSFAFPDDPETPILKDLDFTVPAGSKLGILGETGAGKSTLVSLISRFYDPTVGHVLIDGIDARDWPLATLRSQVCIVAQDTFLFSDTIGGNIGFGASGDSDDRYIQKMAQIAGADNFIRSMPQGYDTVVGERGVGLSGGQKQRLSLARALADNPSILIMDDTTSAVDMETEAEIQKHLKEMDGGKTIVAIAHRISSVKDSDLILVLEHGRIVERGTHAELVAAHGRYWDIYHKQLGLQSGVAQGF

SEQ ID NO: 41 - DNA, 2766 bp, synthetic construct
  misc_feature 1..2766: note = Synthetic polynucleotide
  source 1..2766: mol_type = other DNA
atgacggctaacagagatgccgctctttcctcccaccgtcatcctggttgtgctcagagacctagaacacctaccttcgcttctagctctcaaagaagatctgcttttggcttcgatgatggcaatttcccagggttgggtgaaagatcacatgctcctggttctagactaggtgccagaagaagagcaaaaactgcaagaggtcttagaggtcatcgtcaaaggggggctggtgccggtttatcacgtcctggcagtgcaagagcccctagtcctcctagacctggtggtccagaaaatcctggcggggtactttcagtcgagttgccaggtttactggcccaacttgccaggtcctttgcattgctattgccagtatatgctcttggctacttaggcctgtcatttagctgggtgctgctggctctggctttgttggcctggtgtcgtagatcaagaggtctgaaggcgttgagactatgtagagctttggccttactggaagatgaagaaagagtggttcgtttaggggtcagagcgtgcgatttgcctgcttgggtacattttccggatactgagagagctgaatggttgaacaagactgtcaaacatatgtggcctttcatatgccaatttatcgagaagctgttcagggaaaccatcgaaccagcagttagaggtgcaaatactcacttgtcaactttctcatttaccaaagtcgatgttggtcaacagccactgagaatcaatggtgttaaggtatacaccgaaaacgtcgacaagagacaaataattttagacctgcagatatcattcgttggaaactgcgagattgatctggagatcaaaagatatttctgtagagctggtgtcaaatcaattcaaatccacggtaccatgagagtcatcttagaacctttgattggtgatatgcctttagttggtgctctgtctattttcttcctgaggaagccacttttagagattaactggactggtcttactaacttgttggacgtacctggtttgaatggtctatcagatacgattatccttgacattatcagcaactacctggtgcttcccaataggattaccgttcctttggtctcagaagtccaaatagcgcaactgaggttcccggtacctaaaggagtcttgcgtatccacttcatagaagcacaagatttgcagggaaaagatacgtacttgaagggtctggttaagggcaagagcgacccttacggcatcattcgtgtaggtaatcaaatattccagtctagggttatcaaggagaacttgagcccgaaatggaatgaggtttatgaagctctggtgtatgaacatccaggtcaagaactagagattgaacttttcgacgaagatcctgacaaggatgatttcctgggctcactgatgattgatctgattgaagtagaaaaagaacgtttgctagatgaatggtttacattagacgaagttccgaaaggtaagctacacttaaggctagaatggctgacgcttatgccaaatgcttcaaatctggacaaagtcttaacagatataaaagctgacaaagaccaagcgaacgatggattatcaagtgctttattgatcttgtatctggattctgcacgtaatctgccctctggtaagaagatctcttccaacccaaatcctgttgttcagatgtccgttggacataaagcgcaagagtccaagatccgttataaaactaacgagcctgtgtgggaagaaaacttcaccttttttattcacaatcctaagagacaagatttggaagttgaagttagagatgagcagcaccagtgttccttaggcaaccttaaggtaccgttatctcaattgttaacctctgaggatatgacagtttctcagcgttttcaactgagtaacagcggtccaaactctactatcaagatgaagattgctttgagagttcttcacttggagaagagggagaggccaccggatcatcaacactcagcccaagttaagagacctagtgtttcaaaggagggaaggaaaacttccataaagagccatatgtcaggttccccaggtcctggaggctcaaatacagctccttcaacaccggtcataggtggaagtgacaagcctggtatggaagaaaaagcgcagcctcctgaagccggtcctcaaggtcttcatgatttgggcagatcctctagctcccttctggcatctcccggtcacatttccgttaaagaacccactccttctattgcgagtgacatctcattgcctattgcaacgcaggaacttcgtcaaagattgaggcagttggaaaacggcactacccttgggcagtcaccacttggtcaaattcagttaactatccgtcattcctcacaaagaaataagctgattgttgtcgtgcatgcatgtagaaatctgatcgctttttccgaggacggatctgatccctacgtaagaatgtacttgttacccgataagaggagatcaggtagaagaaagacgcatgtttcaaaaaagactctgaacccggttttcgaccaatctttcgatttttctgtttctctacctgaggtacaaagaaggacgttagatgttgcagtcaagaactcaggtggtttcttgagcaaggacaaagggttgctgggtaaggtgttggttgcgttggcttcagaggagttggctaaagggtggactcaatggtacgatctgacggaagatggaacaagaccacaagccatgacataa

SEQ ID NO: 42 - protein, 921 aa, Homo sapiens
MTANRDAALSSHRHPGCAQRPRTPTFASSSQRRSAFGFDDGNFPGLGERSHAPGSRLGARRRAKTARGLRGHRQRGAGAGLSRPGSARAPSPPRPGGPENPGGVLSVELPGLLAQLARSFALLLPVYALGYLGLSFSWVLLALALLAWCRRSRGLKALRLCRALALLEDEERVVRLGVRACDLPAWVHFPDTERAEWLNKTVKHMWPFICQFIEKLFRETIEPAVRGANTHLSTFSFTKVDVGQQPLRINGVKVYTENVDKRQIILDLQISFVGNCEIDLEIKRYFCRAGVKSIQIHGTMRVILEPLIGDMPLVGALSIFFLRKPLLEINWTGLTNLLDVPGLNGLSDTIILDIISNYLVLPNRITVPLVSEVQIAQLRFPVPKGVLRIHFIEAQDLQGKDTYLKGLVKGKSDPYGIIRVGNQIFQSRVIKENLSPKWNEVYEALVYEHPGQELEIELFDEDPDKDDFLGSLMIDLIEVEKERLLDEWFTLDEVPKGKLHLRLEWLTLMPNASNLDKVLTDIKADKDQANDGLSSALLILYLDSARNLPSGKKISSNPNPVVQMSVGHKAQESKIRYKTNEPVWEENFTFFIHNPKRQDLEVEVRDEQHQCSLGNLKVPLSQLLTSEDMTVSQRFQLSNSGPNSTIKMKIALRVLHLEKRERPPDHQHSAQVKRPSVSKEGRKTSIKSHMSGSPGPGGSNTAPSTPVIGGSDKPGMEEKAQPPEAGPQGLHDLGRSSSSLLASPGHISVKEPTPSIASDISLPIATQELRQRLRQLENGTTLGQSPLGQIQLTIRHSSQRNKLIVVVHACRNLIAFSEDGSDPYVRMYLLPDKRRSGRRKTHVSKKTLNPVFDQSFDFSVSLPEVQRRTLDVAVKNSGGFLSKDKGLLGKVLVALASEELAKGWTQWYDLTEDGTRPQAMT

SEQ ID NO: 43 - DNA, 1560 bp, synthetic construct
  misc_feature 1..1560: note = Synthetic polynucleotide
  source 1..1560: mol_type = other DNA
atggaaggcgttctttacaagtggaccaattatttgtctggatggcaacctagatggttccttctatgtggtggcatcctttcatattacgattctccagaagatgcgtggaagggatgtaagggttcaattcagatggctgtttgcgaaatccaagtgcattccgtcgataacaccagaatggacctgattattcctggtgaacagtatttctacttgaaggcaaggagtgttgctgaaagacaaaggtggcttgttgccctaggttcagctaaggcttgtttgactgattctaggactcaaaaggaaaaagagttcgctgaaaacacagaaaacctgaaaaccaagatgtctgaactgcgtctttactgcgatttgttggttcaacaagtcgataagacaaaggaagtcacgacaactggtgttagcaattcagaagagggtatcgatgtcggtactctactaaagtctacctgtaatacgttcctgaagaccttagaggaatgtatgcaaattgcaaatgccgctttcacatcagaacttttataccgtactccgccaggttcaccgcaattggctatgttgaaaagctccaaaatgaagcaccctattataccaattcacaatagccttgagaggcagatggaattatctacttgcgagaatgggtctcttaatatggaaatcaatggtgaagaagaaatcctgatgaagaacaagaacagcttgtaccttaagtctgccgagattgattgttccattagctcagaggagaatactgatgacaacattacagtccaaggtgagatcagaaaagaagacggtatggagaacttgaagaaccacgataacaacctgactcaatctggaagtgatagttcctgctccccagaatgcctgtgggaggagggtaaggaagtaattcctacgttctttagcactatgaacacatcattcagtgacattgaactgttggaggactctggcataccgactgaagctttccttgctagctgctatgctgttgttccggttcttgacaagttaggtccgaccgttttcgccccagttaagatggatttggtcggtaacatcaagaaagttaaccaaaagtacatcactaacaaggaagagttcacaacactgcagaagatcgtcctgcatgaggtagaggctgatgtcgctcaagtcagaaacagcgcaacagaagcgttgttgtggttaaagagagggttgaagttcttgaaaggttttttaacggaagtaaaaaatggggagaaggacattcaaactgccttaaacaatgcatatggtaaaactctgagacaacatcatggttgggtcgtcagaggtgtttttgcccttgctttaagggccgctccatcctacgaagacttcgttgccgctttaactgtgaaagaaggagaccaccaaaaagaagccttttctataggaatgcaaagagatctaagcctttatcttcctgcaatggaaaagcaacttgctattctggacaccttatacgaagttcatggcttagagtcagatgaagtcgtctaa

SEQ ID NO: 44 - protein, 519 aa, Homo sapiens
MEGVLYKWTNYLSGWQPRWFLLCGGILSYYDSPEDAWKGCKGSIQMAVCEIQVHSVDNTRMDLIIPGEQYFYLKARSVAERQRWLVALGSAKACLTDSRTQKEKEFAENTENLKTKMSELRLYCDLLVQQVDKTKEVTTTGVSNSEEGIDVGTLLKSTCNTFLKTLEECMQIANAAFTSELLYRTPPGSPQLAMLKSSKMKHPIIPIHNSLERQMELSTCENGSLNMEINGEEEILMKNKNSLYLKSAEIDCSISSEENTDDNITVQGEIRKEDGMENLKNHDNNLTQSGSDSSCSPECLWEEGKEVIPTFFSTMNTSFSDIELLEDSGIPTEAFLASCYAVVPVLDKLGPTVFAPVKMDLVGNIKKVNQKYITNKEEFTTLQKIVLHEVEADVAQVRNSATEALLWLKRGLKFLKGFLTEVKNGEKDIQTALNNAYGKTLRQHHGWVVRGVFALALRAAPSYEDFVAALTVKEGDHQKEAFSIGMQRDLSLYLPAMEKQLAILDTLYEVHGLESDEVV

SEQ ID NO: 45 - DNA, 660 bp, synthetic construct
  misc_feature 1..660: note = Synthetic polynucleotide
  source 1..660: mol_type = other DNA
atggtcaagtacttcttaggtcagtcagtgttgagaagttcttgggatcaggtgtttgcggcattctggcagagatatccgaacccttatagcaagcatgtgcttacggaggacattgtccacagggaagtaactcctgaccagaaactgttgtctagacgtttattgacaaagaccaatcgtatgcctagatgggcagaaaggttgtttcctgctaacgtcgctcacagcgtctacgttttagaggattccattgtcgatccacagaaccaaactatgacgaccttcacctggaacattaaccatgctcgtctgatggtggttgaggaaaggtgtgtatattgcgtcaacagcgataactcaggttggaccgagattaggagagaagcttgggttagctcatcactgtttggagttagtcgtgctgtacaggaatttggattggctcgtttcaagtcaaatgtgactaaaacgatgaagggtttcgagtacattttggccaagctacagggagaagctccaagcaaaactcttgtggaaaccgccaaagaagcgaaagagaaggctaaggaaacagcattagctgccacagaaaaagccaaagatctggcctctaaggctgccacaaagaagcaacaacagcaacaacagtttgtctaa

SEQ ID NO: 46 - protein, 219 aa, Homo sapiens
MVKYFLGQSVLRSSWDQVFAAFWQRYPNPYSKHVLTEDIVHREVTPDQKLLSRRLLTKTNRMPRWAERLFPANVAHSVYVLEDSIVDPQNQTMTTFTWNINHARLMVVEERCVYCVNSDNSGWTEIRREAWVSSSLFGVSRAVQEFGLARFKSNVTKTMKGFEYILAKLQGEAPSKTLVETAKEAKEKAKETALAATEKAKDLASKAATKKQQQQQQFV

SEQ ID NO: 47 - DNA, 570 bp, synthetic construct
  misc_feature 1..570: note = Synthetic polynucleotide
  source 1..570: mol_type = other DNA
atgggtgtttctgtcgatgtccatcaagtgtacaagtacccgtttgaacaagttgtagcctcattcctgcgtaagtatcctaatcctatggacaaaaacgtcatttctgtgaagatcatggaagaaaagagagacgaatctacgggtgttatctacaggaagcgtattgccatctgccaaaacgttgttccggaaattctgagaaagagcttatctactttagtcatcttgtgttggaaaaaggtgtcaattctgaaggttccaaacatccaactagaagaagaaagctggcttaatcctagagaacgtaacatggctatacgttctcattgcttaacttggacgcaatacgccagtatgaaggaggaaagcgtatttcgtgaatctatggagaaccctaactggacagagttcatccagagaggtagaatctccattactggtgtaggtttccttaactgcgttctggaaacttttgcatctacattcctaagacaaggagctcaaaagggaatcagaatcatggaaatgcttcttaaggaacaatgtggtgctccactggctgaataa

SEQ ID NO: 48 - protein, 189 aa, Homo sapiens
MGVSVDVHQVYKYPFEQVVASFLRKYPNPMDKNVISVKIMEEKRDESTGVIYRKRIAICQNVVPEILRKSLSTLVILCWKKVSILKVPNIQLEEESWLNPRERNMAIRSHCLTWTQYASMKEESVFRESMENPNWTEFIQRGRISITGVGFLNCVLETFASTFLRQGAQKGIRIMEMLLKEQCGAPLAE

SEQ ID NO: 49 - DNA, 1152 bp, synthetic construct
  misc_feature 1..1152: note = Synthetic polynucleotide
  source 1..1152: mol_type = other DNA
atggcaactgcttcaccaccgttcatctcaaccttgagcttcacacacagcagcttcaagacctcatcttcttcttccttttcacctaaactgttgcgtccattgctttccttttccgtaaaggctagtaggaagcaagtcgagatcgtattcgatcctgatgagagactgaacaaaattggagatgatgtcgataaggaagcacctctgtcaagactgaaactgttctcaccatgcaagatcaacgtatttctgagaattactggcaagagggaagacggttttcatgacttagcctccctgtttcatgtgatttctcttggagatactatcaagttctcactaagcccttctaagtccaaggacaggctttctaccaacgttcagggtgttccagtagacggaagaaatttgatcatcaaagcattgaatctgtacagaaagaagacgggtagcaataggttcttttggattcacttggataagaaagtcccaactggtgcaggcctaggaggaggcagctctaatgccgcaactgcattatgggctgcgaatgagctaaacggagggttggtcactgaaaatgagctgcaagattggtcctcagaaattgggagcgatatccctttcttcttctctcatggtgctgcctactgtactggtagaggtgagatcgtccaagacttacctccaccattcccgttggatctgcctatggtgttgatcaaaccacgtgaagcctgttcaaccgcagaggtctacaagaggttaagattggatcaaactagcaacatcaaccctctaacattgctggaaaacgtcactagtaatggtgtttcccaatctatctgtgttaatgaccttgaaccacctgcattctctgtattaccatctttaaaaaggctaaaacagaggatcatagcctcaggaagaggcgaatatgatgccgtcttcatgtctggttcaggatctaccatcattggtattggttccccagatcctccacagttcatctatgacgatgaagagtacaagaatgtgttcttgtcagaagcaaacttcatgacaagggaagcgaacgaatggtacaaagagccagctagtgccaatgctactacttcatctgctgagtccagaatggacttccaataa

SEQ ID NO: 50 - protein, 383 aa, Arabidopsis thaliana
MATASPPFISTLSFTHSSFKTSSSSSFSPKLLRPLLSFSVKASRKQVEIVFDPDERLNKIGDDVDKEAPLSRLKLFSPCKINVFLRITGKREDGFHDLASLFHVISLGDTIKFSLSPSKSKDRLSTNVQGVPVDGRNLIIKALNLYRKKTGSNRFFWIHLDKKVPTGAGLGGGSSNAATALWAANELNGGLVTENELQDWSSEIGSDIPFFFSHGAAYCTGRGEIVQDLPPPFPLDLPMVLIKPREACSTAEVYKRLRLDQTSNINPLTLLENVTSNGVSQSICVNDLEPPAFSVLPSLKRLKQRIIASGRGEYDAVFMSGSGSTIIGIGSPDPPQFIYDDEEYKNVFLSEANFMTREANEWYKEPASANATTSSAESRMDFQ

SEQ ID NO: 51 - DNA, 2226 bp, synthetic construct
  misc_feature 1..2226: note = Synthetic polynucleotide
  source 1..2226: mol_type = other DNA
atggcaacaggtgttttacctgcgccagtaagcggcatcaagatcccagattctaaggtagggttcggaaagtccatgaatcttgtgagaatttgcgacgtccgtagccttaggtcagctagaagaagagtttcagtgattcgtaactctaaccaaggttctgacttagctgagttacaacctgcttcagaaggatcaccactgcttgttcctaggcagaagtattgtgaatcccttcataaaactgtacgtagaaaaacaaggactgttatggtaggtaatgtcgctctgggttctgaacatcctatcagaatccaaactatgactacttcagacacgaaagatattacgggaaccgtagatgaggttatgaggatagcagataaaggtgcagacatcgttcgtatcacagtccaggggaaaaaagaggctgatgcgtgttttgaaatcaaggacaaactggtgcaacttaactacaacatccctttggttgcggatattcattttgcacctactgttgctctgagagtcgctgaatgtttcgacaagataagagtaaatccaggtaattttgccgatagaagggctcagttcgagacaatagactacactgaagatgaataccagaaagaacttcaacacattgaacaggtctttacgcctttggttgaaaagtgcaagaagtatggtagagcgatgagaattgggacgaatcatggaagcctttcagacagaatcatgtcttactacggtgattctcctagaggcatggttgaaagtgcttttgagttcgcaaggatatgcaggaagttagactatcataactttgtcttctctatgaaagcatcaaatcctgtcatcatggttcaagcttaccgtcttttggttgcagagatgtacgtccacggttgggattatccgcttcacttgggtgtaaccgaagctggagaaggtgaagacggtaggatgaagagcgctataggaattggtacattattacaagatggtttgggtgataccattagggtgtcattaacagaacctccagaagaggaaatagacccttgtcgtcgtctagctaacttaggtaccaaagcagctaaactgcagcaaggtgttgctccattcgaagagaaacataggcactacttcgatttccaaaggagaactggcgatcttccagtccagaaggaaggtgaggaagtcgattaccgtaatgtccttcatagggatggatctgttttgatgagcattagccttgaccaattgaaggctccagaactgctatacagaagccttgctactaagttggttgtaggtatgccattcaaagatttggcaactgtggactcaatcttgcttagggagctgccacctgtagacgatcaggtcgctagattggcattaaaacgtctaatagatgtgagcatgggtgtaatcgctccgttatctgaacagttgactaagccacttccaaatgctatggtgttagtgaacttgaaggagttatcaggcggagcctataagctgttacctgaaggaacgagattagttgtctctctaagaggagatgaaccgtacgaagaattagagatcttaaagaacatagatgccacgatgatcttacacgatgtgccatttaccgaagataaggtatcaagagtccatgctgcaagaagattgttcgagtttctgtcagagaatagtgtgaatttcccggtaattcaccatatcaacttcccaacgggtatacatagggatgagctggtcatccacgcgggtacatacgcaggcggtctacttgtagacgggttaggtgatggcgtgatgctggaagcaccagatcaagacttcgattttctgagaaatacaagctttaacttgcttcaaggatgtaggatgcgtaataccaagacagaatatgtgagctgtccatcatgtggtagaacgttatttgacttacaagagatttccgccgaaattagggagaaaactagccatctaccaggtgttagtatcgctattatgggctgcatcgttaatggcccaggtgagatggccgatgctgactttggatatgtaggtggttctccggggaagatagacttgtatgtcggcaagactgttgttaagcgtgggatcgctatgactgaggctactgacgccttgatcggtcttatcaaggaacatgggagatgggttgatccacctgtcgccgacgaataa

SEQ ID NO: 52 - protein, 741 aa, Arabidopsis thaliana
MATGVLPAPVSGIKIPDSKVGFGKSMNLVRICDVRSLRSARRRVSVIRNSNQGSDLAELQPASEGSPLLVPRQKYCESLHKTVRRKTRTVMVGNVALGSEHPIRIQTMTTSDTKDITGTVDEVMRIADKGADIVRITVQGKKEADACFEIKDKLVQLNYNIPLVADIHFAPTVALRVAECFDKIRVNPGNFADRRAQFETIDYTEDEYQKELQHIEQVFTPLVEKCKKYGRAMRIGTNHGSLSDRIMSYYGDSPRGMVESAFEFARICRKLDYHNFVFSMKASNPVIMVQAYRLLVAEMYVHGWDYPLHLGVTEAGEGEDGRMKSAIGIGTLLQDGLGDTIRVSLTEPPEEEIDPCRRLANLGTKAAKLQQGVAPFEEKHRHYFDFQRRTGDLPVQKEGEEVDYRNVLHRDGSVLMSISLDQLKAPELLYRSLATKLVVGMPFKDLATVDSILLRELPPVDDQVARLALKRLIDVSMGVIAPLSEQLTKPLPNAMVLVNLKELSGGAYKLLPEGTRLVVSLRGDEPYEELEILKNIDATMILHDVPFTEDKVSRVHAARRLFEFLSENSVNFPVIHHINFPTGIHRDELVIHAGTYAGGLLVDGLGDGVMLEAPDQDFDFLRNTSFNLLQGCRMRNTKTEYVSCPSCGRTLFDLQEISAEIREKTSHLPGVSIAIMGCIVNGPGEMADADFGYVGGSPGKIDLYVGKTVVKRGIAMTEATDALIGLIKEHGRWVDPPVADE

SEQ ID NO: 53 - DNA, 1404 bp, synthetic construct
  misc_feature 1..1404: note = Synthetic polynucleotide
  source 1..1404: mol_type = other DNA
atggccttgaacttgttgtctccagctgagttaaaatccgtctcttttctagactccactagatcctctcaacttccgaagttgccggggtctttctcattgaagagaaaggagtttgggaggaaagtgcaatgtagtgctcaggctccgccgccagcatggcctggtagagcagtagcagaaccaggtagaaaaacgtgggatggtccaaagcctatttccatcgttggttctaccgggtcaataggaacccagactcttgatatagttgctgaaaacccggataaattccgtgttgttgcattagctgcaggttcaaatgtgacattacttgctgaccaagtcaagacgtttaagcctcaattagtgtccgttagagatgaatcattggttgatgagctgaaagaagctttagcggatgtggatgaaaagccagaaatcatccctggtgaacaagggattgtggaagtggccagacatccagatgcagtaagcgtggttactggtattgtaggatgtgccggtttgaagccgactgtagcagcgatagaagctggtaaagacatctgcttggctaataaggaaactttgattgccggtggcccttttgtgcttcctcttgcgcacaagtataacgtgaagatacttcctgctgattccgaacattctgccatcttccaatgtatccaaggattaccggaaggtgcattacgtagaatcatcttgacagcttcaggaggagctttcagagattggcctgtcgaaaaactgaaagacgtaaaggtcgcagatgcattgaaacatccaaattggaacatgggtaagaagatcactgtcgactccgccaccctgttcaacaaggggttggaagtcatcgaggcccactatctgtttggcgccgattatgataacatcgagatcgtgatccatccgcaatccatcatccattctatggttgaaacagaggactcctcagttttggcacaattaggatggcctgacatgaggattcccatactgtacaccatgtcttggccagatagaatctattgttctgaaatcacatggcctagacttgatctttgcaagctggggtcattaaccttcaaagcaccagataacgtcaagtatccatctatggaactagcgtatgcagctggtagggctgggggaactatgactggagttttgtccgcagcaaatgaaaaggctgttgagatgttcattgacgaacagataagttatcttgacatcttcaagattgtcgaattgacctgtgataagcatagagctgaattggttacgtcaccttctctggaagagattatccattacgatctttgggcaagggaatatgccgccagcttacaaccttcatcagggttgtctccggttcttgcttaa

SEQ ID NO: 54 - protein, 467 aa, Ricinus communis
MALNLLSPAELKSVSFLDSTRSSQLPKLPGSFSLKRKEFGRKVQCSAQAPPPAWPGRAVAEPGRKTWDGPKPISIVGSTGSIGTQTLDIVAENPDKFRVVALAAGSNVTLLADQVKTFKPQLVSVRDESLVDELKEALADVDEKPEIIPGEQGIVEVARHPDAVSVVTGIVGCAGLKPTVAAIEAGKDICLANKETLIAGGPFVLPLAHKYNVKILPADSEHSAIFQCIQGLPEGALRRIILTASGGAFRDWPVEKLKDVKVADALKHPNWNMGKKITVDSATLFNKGLEVIEAHYLFGADYDNIEIVIHPQSIIHSMVETEDSSVLAQLGWPDMRIPILYTMSWPDRIYCSEITWPRLDLCKLGSLTFKAPDNVKYPSMELAYAAGRAGGTMTGVLSAANEKAVEMFIDEQISYLDIFKIVELTCDKHRAELVTSPSLEEIIHYDLWAREYAASLQPSSGLSPVLA

SEQ ID NO: 55 - DNA, 1401 bp, synthetic construct
  misc_feature 1..1401: note = Synthetic polynucleotide
  source 1..1401: mol_type = other DNA
atggcgatatctctacaactgtgtagacttagtttgcgtactgatttgtttagcagagataacaactcatcaccattgttgagaagaaaacctttcagcatcagatgctccgctgctgcggcttccgatgatgaatcctctagcgcatctgtcgcaatggatagcgactttgatgctaaggtctttagacataatttgactcgttcaaaaaactacaacagacgtggttttggtcataaggaagaaactttgcaactaatgagtcaggaatatacgtcagacatcatcaagactttgaaggaaaacggtaacgagtacacctggggaaatgtaaccgtcaagttagccgaagcttatggtttctgttggggtgtcgaaagagctgtccagatagcttatgaagcgagaaaacagttccctgacgaaaagatctggatcactaacgagatcatccataatccaacagtcaacaagagattagaggaaatgaatgtcgaaaacattcctcttgaagaaggtaggaaacagttcgaggttgtcaacaacggtgatgttgtgatccttcctgcttttggagccgctgttgatgaaatgctaacactgtcaaataagaatgtgcaaattgtcgatacaacttgtccttgggtgtcaaaagtgtggaataccgtcgagaagcacaagaagggagattacacgagcattatccacggcaagtattctcatgaagagactattgctacagcatcttttgctggtacatatattattgtgaagaacatgaaagaagcaatgtacgtatgtgactatattttaggaggtcagctaaatgggtcatccagtacgaaggaagagttccttaaaaagtttaagaacgcagtaagcaagggttttgatcccgatgtagatctggttaaagtcggtattgctaaccaaacgacgatgcttaaaggtgaaactgaagacataggcaaattagtcgaaaagacgatgatgcaaaggtatggcgtggaaaatgttaacgaccattttatatcctttaatacgatttgtgatgccactcaagaacgtcaagatgcgatgttcaacttagtggaagaaaagctggacctgatactggtaattggtggttggaatagctctaacaccagtcatctacaggaaattgcagaacttagaggtatcccttcctattggattgattctgaacaacgtattggtccaggtaatagaattgcgtacaaactgaaccacggtgaattggttgagaaggagaattttttaccggaaggtccgatcactattggggttacttcaggagcaagcaccccagataaagtcgtggaagatgcgttggttaaggtattcgatatcaagagagaagaagccttgcaattggcgtaa

SEQ ID NO: 56 - protein, 466 aa, Ricinus communis
MAISLQLCRLSLRTDLFSRDNNSSPLLRRKPFSIRCSAAAASDDESSSASVAMDSDFDAKVFRHNLTRSKNYNRRGFGHKEETLQLMSQEYTSDIIKTLKENGNEYTWGNVTVKLAEAYGFCWGVERAVQIAYEARKQFPDEKIWITNEIIHNPTVNKRLEEMNVENIPLEEGRKQFEVVNNGDVVILPAFGAAVDEMLTLSNKNVQIVDTTCPWVSKVWNTVEKHKKGDYTSIIHGKYSHEETIATASFAGTYIIVKNMKEAMYVCDYILGGQLNGSSSTKEEFLKKFKNAVSKGFDPDVDLVKVGIANQTTMLKGETEDIGKLVEKTMMQRYGVENVNDHFISFNTICDATQERQDAMFNLVEEKLDLILVIGGWNSSNTSHLQEIAELRGIPSYWIDSEQRIGPGNRIAYKLNHGELVEKENFLPEGPITIGVTSGASTPDKVVEDALVKVFDIKREEALQLA

SEQ ID NO: 57 - DNA, 1215 bp, synthetic construct
  misc_feature 1..1215: note = Synthetic polynucleotide
  source 1..1215: mol_type = other DNA
atggctccagcggctgccgactcaattaagccgagagatgtctgtatcgttggtgtatcaagaacaccaatgggagggtttctggggagcatatcttcactacctgctaccaagctgggttctatagcgatcgaaagtgctctaaaacgtgcaaatgttgacccttcacatgttcaggaagtttttttcggcaacgtgttatccgcgaaccttggacaggcaccggctcgtcaagctgctcttggagcaggcatcccaaacaccgtaatttgtactaccgtcaacaaggtatgtgcatctgggatgaaggctgctatgttagctgcgcaatccatacaattggggattaacgacgtggttgttgctggtggaatggaatctatgtccaacgtaccgaaatacctagcggaagcaagaaaaggttctagacttggtcacgattctcttgtcgatggtatgttgaaggacggtttgtgggacgtttacaatgactatggtatgggagtttgtgcggaattatgtgccgaacatcatagcgttactcgtgaagaacaggataactatgcagtacaatccttcgagagaggtatcgccgcaaaggactctggtgctttctcctgggaaatcgtcccagttgaagtcagcggcggtagaggtggcccatcaactatcgtggacaaagatgaaggcctggggaaattcgatgctgcgaaactgcgtaagttgaggcctagtttcaaggaaacaggtggttctgttacagcgggtaatgcttcttccatttcagatggcgctgcggctctggtgttggttagtggtgagaaggctcttaagctgaggttggaagttattgcgaagattaccggatacgcagatgctgctcaggctccagaattgttcacgacggcccctgctattgcaatccctaaggccatatccaatgcaggtttggaagcctcccaaatcgactattacgagattaacgaggcttttgcagtagttgctcttgccaaccagaagttgctgtcacttaatccagagaaagtcaatgtgcacggtggggctgtttctttgggtcatccactaggctgctctggcgccaggatactggtaacactattaggtgtccttagacagaagggaggaaaatatggtgtcggaggggtttgcaacggtggtggtggggcatcagctgtagtgatagagcttctataa

SEQ ID NO: 58 - protein, 404 aa, Picrorhiza kurrooa
MAPAAADSIKPRDVCIVGVSRTPMGGFLGSISSLPATKLGSIAIESALKRANVDPSHVQEVFFGNVLSANLGQAPARQAALGAGIPNTVICTTVNKVCASGMKAAMLAAQSIQLGINDVVVAGGMESMSNVPKYLAEARKGSRLGHDSLVDGMLKDGLWDVYNDYGMGVCAELCAEHHSVTREEQDNYAVQSFERGIAAKDSGAFSWEIVPVEVSGGRGGPSTIVDKDEGLGKFDAAKLRKLRPSFKETGGSVTAGNASSISDGAAALVLVSGEKALKLRLEVIAKITGYADAAQAPELFTTAPAIAIPKAISNAGLEASQIDYYEINEAFAVVALANQKLLSLNPEKVNVHGGAVSLGHPLGCSGARILVTLLGVLRQKGGKYGVGGVCNGGGGASAVVIELL

SEQ ID NO: 59 - DNA, 1197 bp, synthetic construct
  misc_feature 1..1197: note = Synthetic polynucleotide
  source 1..1197: mol_type = other DNA
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 60 - protein, 398 aa, Saccharomyces cerevisiae
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 61 - DNA, 900 bp, synthetic construct
  misc_feature 1..900: note = Synthetic polynucleotide
  source 1..900: mol_type = other DNA
atggccaatagaggagaaccggatccaaagaagagcaccgaatcaatctgctctctgactaaaccgcagctttattcattatatgatgatgacgtcgtgagatcggaagataacgaaatttacgaagaattgaaacgctctgtctctatagattctaccaaatatagtagagatcaaactattgattcgactttttacttggcccataaggttggcggctcgctgccaaggaacacagtatcttctaataatttggaaaggattttatcagcatcatccattcacgaaaactttccgtcaagaaccagacaaactcgtcaaaatattcttcattacctgcaagctgtattaattttgtcattgagcggtttcgcatatcatgaactgtccagaaacttgcacgataatcatctcttacatccagattttgcatcgagaccccttctactgggtgtcaaactttgtaactggctatccaatggggttctgccaaactggttaggctacggtgttgaaggactgttatttggttctgttgtaccaattctggataatatttttcaaacagaggttgttaagagctctgtccaccatgacagtttgacctctgttattagaagtatcaatgccatgttgggagtcacatttgggatcaggaaaatacagtggaactcctctttgcaggcggcaggggcatgggggcttctgaatattatactgtggttgttttttgacggctcaatatccatgttaatgagctgcatctgcattggggtgggttgttgtatctcttgctacaaagatattatcgacggttcccaatttttatatttcatggatttttatttccttggttctctaatgtttggaaaactaggaagatatctatattctcattaa

SEQ ID NO: 62 - protein, 299 aa, Saccharomyces cerevisiae
MANRGEPDPKKSTESICSLTKPQLYSLYDDDVVRSEDNEIYEELKRSVSIDSTKYSRDQTIDSTFYLAHKVGGSLPRNTVSSNNLERILSASSIHENFPSRTRQTRQNILHYLQAVLILSLSGFAYHELSRNLHDNHLLHPDFASRPLLLGVKLCNWLSNGVLPNWLGYGVEGLLFGSVVPILDNIFQTEVVKSSVHHDSLTSVIRSINAMLGVTFGIRKIQWNSSLQAAGAWGLLNIILWLFFDGSISMLMSCICIGVGCCISCYKDIIDGSQFLYFMDFYFLGSLMFGKLGRYLYSH

SEQ ID NO: 63 - DNA, 990 bp, synthetic construct
  misc_feature 1..990: note = Synthetic polynucleotide
  source 1..990: mol_type = other DNA
atgggcaagtaccttcttcttagattatactacttgcatggaggtcagagaaagtggctgtcaagctggcttcaaaccgtagccttcccttttctgctaatccctatttccgtttcctggtttaaaagcaagtccaagtcacatgattctaggtctatctctgctatagacgttaatccaacgacggataggaagctgagatttggtgggttctcacctaagctattcatctcctgtatcttcctgggtatcatcgtggggttggattccttcctgtacgcctatggtgtttcatacttaccagtatcaacttcttcactattgatgtctactcaattggcttttactgccgcatttgcgcttttactggtgaggcagaaattcactccatatagcataaactccgttgtcctgcttactctaggtgctgttgtgctggcatttcataccaacggtgataagcctattggagtctcaaaggaccagtactttctgggatttttcgtaacccttggtgcagccgcgctgttcggttttatgctgccgttcattgagctggtctataggaaagcatgtgaagctgttacctatgatctggtcatgagaatgcagttcatcatctcaatggtagcaacggtgttttgtacaatagctatgttaataaataaggatttccaggcaatttctagggaagcgaaaggattcgagttaggtgaaactaagtactacatagttctgatctttactgccgtcagcatgcaatgcgccgtggtaggcacacttggcgtcatacactgtgcttcatctttgttctccggcgttttaatgactcttttgttgcctatccaacagatatgcgctatcttctttttcaacgagaaattctcagccgaaaaagggatgtcattaggtcttagtatttggggatttgcttcctacttctatggtgaatacaagcaaacagagaaaaagaccaaccagcataaatga

SEQ ID NO: 64 - protein, 329 aa, Papaver somniferum
MGKYLLLRLYYLHGGQRKWLSSWLQTVAFPFLLIPISVSWFKSKSKSHDSRSISAIDVNPTTDRKLRFGGFSPKLFISCIFLGIIVGLDSFLYAYGVSYLPVSTSSLLMSTQLAFTAAFALLLVRQKFTPYSINSVVLLTLGAVVLAFHTNGDKPIGVSKDQYFLGFFVTLGAAALFGFMLPFIELVYRKACEAVTYDLVMRMQFIISMVATVFCTIAMLINKDFQAISREAKGFELGETKYYIVLIFTAVSMQCAVVGTLGVIHCASSLFSGVLMTLLLPIQQICAIFFFNEKFSAEKGMSLGLSIWGFASYFYGEYKQTEKKTNQHK

SEQ ID NO: 65 - DNA, 1047 bp, synthetic construct
  misc_feature 1..1047: note = Synthetic polynucleotide
  source 1..1047: mol_type = other DNA
atgaacacatacttgttgctattcaacggaatcctactggcagttagctctatcgcgggtccgctgctgctgagattgtacttcatacatggcggtaagaggatctggctgtcttcatgtctggaaactgcaggttttccagtccttatctttccactgtggctgagctacttcctgaggagaagaggattcatcaagggcgatgatgacgaaaaacctaagaagttgttcaccattaccctaccccttttcatagcaagtgctgtcatcggtctggttaccggtcttgacgactacctgtacacctacggagtttcactgcttcctatctctactgcgaccatcatcatgtcaacccacctggcatttactgccggttttgcattggtcatggttaaacagaagttcacttcattctcagttaatgctgttgtcttgcttacgattggtgcgattctgctgggtttgcatggtaatggcgacaaacctgtaaacgaaagcaaaaaggactattacctgggtttcttgatcactatagctgcttcagtcttcaacggcttaatgctgcctatggtggaattgatgtacatgaagtcaaagcagacaattacctactcacttgtgattgaattgcaaatggttatctctggtttcgccactctgttctgtacgattgggatgatagccaacaacgatttcaaggttattcctagagagggtagagagtacggattaggcgaaatcaactactacattgttcttgtagcttctgctatcacatggcagatgtacttcgtggggactgtcggagtcattttctgcagcacatccctgcatgcaggtgtgatttccgtagttgttcttcctttaactgaaatcctaagcgttgtcttctaccatgaaagcttcaaagcggagaaaggaatcgcactgttcctttcactttgggggtttatttcatacttctacttggagatcaaagcttcaagaaagccaaagaaacagtgctctgaattggaacaaggtggtctgactgtatcctcataa

SEQ ID NO: 66 - protein, 348 aa, Papaver somniferum
MNTYLLLFNGILLAVSSIAGPLLLRLYFIHGGKRIWLSSCLETAGFPVLIFPLWLSYFLRRRGFIKGDDDEKPKKLFTITLPLFIASAVIGLVTGLDDYLYTYGVSLLPISTATIIMSTHLAFTAGFALVMVKQKFTSFSVNAVVLLTIGAILLGLHGNGDKPVNESKKDYYLGFLITIAASVFNGLMLPMVELMYMKSKQTITYSLVIELQMVISGFATLFCTIGMIANNDFKVIPREGREYGLGEINYYIVLVASAITWQMYFVGTVGVIFCSTSLHAGVISVVVLPLTEILSVVFYHESFKAEKGIALFLSLWGFISYFYLEIKASRKPKKQCSELEQGGLTVSS

SEQ ID NO: 67 - DNA, 1554 bp, synthetic construct
  misc_feature 1..1554: note = Synthetic polynucleotide
  source 1..1554: mol_type = other DNA
atgcagttcaactgggacattaagactgtagcaagcattctttctgcgctaactttagctcaagcctccgatcaagaagcgattgcaccagaagatagccacgtagtgaagcttactgaagcaacttttgaatcattcatcacctctaatcctcatgtgttggcggagttttttgccccttggtgcggacattgcaagaagttaggtcctgaattggtctctgctgctgagatcctgaaagacaatgagcaagtgaagattgcccaaattgattgtacggaggaaaaagagttgtgccagggctacgagataaaaggctatccaacattaaaggtgttccatggcgaagttgaggtccctagtgactaccagggtcagagacaatctcaaagcatagtgagctatatgctgaagcagtcattgccacccgtgtctgagataaacgccaccaaggatttagacgatactatagctgaggcaaaagaaccagttatcgtgcaagtattgcctgaagatgcctcaaacttggaatccaatacgactttttacggcgttgctggaacattgcgtgaaaagttcacattcgtttcaaccaaatcaacggattacgccaagaaatatacgtctgacagtacaccagcatacttgttggttagaccaggagaggagccatccgtctattccggagaagagcttgatgaaacacatctggttcactggattgatattgaatccaagccgttatttggtgatattgacggttccaccttcaaatcctacgctgaagcaaacattccattggcctactacttctacgagaacgaagagcaaagagctgctgctgccgatattatcaaacctttcgctaaagaacaacgtggtaagatcaactttgtgggcttagatgccgttaagtttggaaaacatgctaaaaacctgaacatggatgaggaaaagcttcctttgtttgtcatacacgatttagtatctaacaagaagttcggtgttccacaagatcaagaattaactaataaagacgtcacagaattgatagagaagttcatcgctggcgaagcagaaccgattgttaagtcagaacctattcccgaaatacaggaggagaaagtcttcaaattggtgggaaaggctcacgatgaagttgtgttcgacgaatcaaaggacgtgcttgtgaagtattatgctccttggtgtggtcactgtaagaggatggcccctgcttatgaggaacttgcgacgctttacgcgaatgacgaggacgcctcctctaaggtggtcatagctaaactggaccacacgctaaacgatgtagacaatgtcgatatccaaggataccctaccttgatcctatatccagctggcgacaagagcaaccctcaattgtacgatgggtcacgtgacttggaatctcttgcggagtttgtcaaggaaaggggcacacataaggttgacgctttagctctaagacctgtggaagaggaaaaagaagctgaggaagaagccgaaagcgaggctgatgctcatgatgagctgtaa

SEQ ID NO: 68 - protein, 517 aa, Pichia pastoris
MQFNWDIKTVASILSALTLAQASDQEAIAPEDSHVVKLTEATFESFITSNPHVLAEFFAPWCGHCKKLGPELVSAAEILKDNEQVKIAQIDCTEEKELCQGYEIKGYPTLKVFHGEVEVPSDYQGQRQSQSIVSYMLKQSLPPVSEINATKDLDDTIAEAKEPVIVQVLPEDASNLESNTTFYGVAGTLREKFTFVSTKSTDYAKKYTSDSTPAYLLVRPGEEPSVYSGEELDETHLVHWIDIESKPLFGDIDGSTFKSYAEANIPLAYYFYENEEQRAAAADIIKPFAKEQRGKINFVGLDAVKFGKHAKNLNMDEEKLPLFVIHDLVSNKKFGVPQDQELTNKDVTELIEKFIAGEAEPIVKSEPIPEIQEEKVFKLVGKAHDEVVFDESKDVLVKYYAPWCGHCKRMAPAYEELATLYANDEDASSKVVIAKLDHTLNDVDNVDIQGYPTLILYPAGDKSNPQLYDGSRDLESLAEFVKERGTHKVDALALRPVEEEKEAEEEAESEADAHDEL

SEQ ID NO: 69 - DNA, 1584 bp, synthetic construct
  misc_feature 1..1584: note = Synthetic polynucleotide
  source 1..1584: mol_type = other DNA
atgagaatcgtcaggagtgttgcaatcgctattgcatgtcactgtataactgctctagccaatcctcaaatcccgtttgatgggaattatactgaaatcatcgtcccggatactgaggttaacataggacagattgtggacattaatcatgaaatcaagccaaaattagtggagttggttaacactgacttttttaagtactacaagctgaacttgtggaaaccatgccctttctggaacggagatgaagggttctgtaagtacaaggattgttctgtcgacttcatcacagattggtcccaagtccctgatatttggcaaccagatcaattgggtaagttaggcgataacactgtccataaggacaaaggtcaggatgagaacgaattaagctctaacgattactgtgccttagacaaggatgatgacgaagacctggtgtacgtcaacttgatcgataatccggagaggttcactggatacggtggtcaacaatcagaatccatatggacagcagtgtacgatgaaaactgctttcaacctaatgaaggctctcagttaggccaagtagaagacctgtgtcttgagaagcagatcttctacaggctggtttctggtttacactcatcaatttctacccatcttaccaacgaataccttaatttaaagaatggtgcctatgaaccaaacttgaagcaatttatgattaaggtcggttactttacggagaggattcagaacttacaccttaactatgtacttgttttaaagtccttaattaagcttcaggagtacaacgtcattgacaacttgcctttggatgattctctgaaagccggattatctggtttaatcagccaaggtgcacaaggtatcaaccaatcttccgatgactacctgttcaatgagaaggttctgttccaaaacgaccagaatgatgacctaaagaacgaatttcgtgataagttccgtaacgtgacgagactaatggattgtgtccattgtgaaagatgcaaactgtggggtaaactgcagaccactggatacggaactgctctgaagattctgtttgacttgaagaatccgaatgatagtattaacctgaaaagagtagagctggttgccttggtgaataccttccacagactttctaagtctgtcgaaagcatcgagaatttcgagaagttgtacaagattcaacctcctacacaggacagagcatctgcttcttctgaatccctgggtttatttgataatgaagacgagcagaacttattaaacagcttctctgttgatcaggcagttattagttcaaaggaggctcccgaagagatcaaaagcaaacctgttggtaaggctgcttacaagcaaaactcttgtcccagtctaggaagcaagtcaattaaggaggcctttcacgaagagcttcatgcctttatcgatgccataggtttcatcttaaactcctataggactttgccaaagctactgtatactctgtttttggtgaagtcatcagagttgtgggacatcttcattggaactcagagacacagggatacgacgtatagagtcgatctttaa

SEQ ID NO: 70 - protein, 527 aa, Pichia pastoris
MRIVRSVAIAIACHCITALANPQIPFDGNYTEIIVPDTEVNIGQIVDINHEIKPKLVELVNTDFFKYYKLNLWKPCPFWNGDEGFCKYKDCSVDFITDWSQVPDIWQPDQLGKLGDNTVHKDKGQDENELSSNDYCALDKDDDEDLVYVNLIDNPERFTGYGGQQSESIWTAVYDENCFQPNEGSQLGQVEDLCLEKQIFYRLVSGLHSSISTHLTNEYLNLKNGAYEPNLKQFMIKVGYFTERIQNLHLNYVLVLKSLIKLQEYNVIDNLPLDDSLKAGLSGLISQGAQGINQSSDDYLFNEKVLFQNDQNDDLKNEFRDKFRNVTRLMDCVHCERCKLWGKLQTTGYGTALKILFDLKNPNDSINLKRVELVALVNTFHRLSKSVESIENFEKLYKIQPPTQDRASASSESLGLFDNEDEQNLLNSFSVDQAVISSKEAPEEIKSKPVGKAAYKQNSCPSLGSKSIKEAFHEELHAFIDAIGFILNSYRTLPKLLYTLFLVKSSELWDIFIGTQRHRDTTYRVDL

SEQ ID NO: 71 - DNA, 996 bp, synthetic construct
  misc_feature 1..996: note = Synthetic polynucleotide
  source 1..996: mol_type = other DNA
atgcctgttgattcttctcataaaacagcatcaccgttgcctccaagaaaacgtgctaaaactgaagaagagaaagagcaaagaagggtcgaaagaatcctgcgtaacagaagagctgctcacgcctcaagggagaaaaaaagacgtcacgtagagtttctggaaaaccacgttgttgatcttgaatctgcattacaagagagcgctaaagcaactaacaagttgaaggaaattcaggacattatcgtctctagacttgaagcacttggtggtacagtgtctgatctggacctgacagtacctgaggtagacttcccaaaatcctctgacttggaaccgatgagtgacttgagcactagctctaaaagtgaaaaggcatctaccagcacaagaagatcattgaccgaggaccttgacgaggatgacgttgctgaatacgatgatgaagaagaagatgaagagctgccaagaaagatgaaggtgttaaacgataagaacaagtcaacttccattaagcaagagaagctgaacgagttgccatcacctttgtcatccgatttttctgatgttgacgaagagaaatccacgctaacccaccttaaactacagcagcagcaacagcaaccagttgacaactacgtctcaacacctctgagcttgcctgaagattcagtcgatttcattaatcctggtaacctgaagatcgaatctgacgagaattttttactgagttcaaacacattacagatcaaacacgaaaacgacacggactacatcacaacagctccatcaggcagcataaatgattttttcaactcatatgacatctcagaaagcaatcgtctacatcatcctgcagttatgacagatagcagtttgcatatcacagctggttctatcggctttttcagccttatcggcggcggtgaatcctctgttgcaggtagacgttcctctgtggggacttaccaactgacttgcatagccattcgttaa

SEQ ID NO: 72 - protein, 331 aa, Pichia pastoris
MPVDSSHKTASPLPPRKRAKTEEEKEQRRVERILRNRRAAHASREKKRRHVEFLENHVVDLESALQESAKATNKLKEIQDIIVSRLEALGGTVSDLDLTVPEVDFPKSSDLEPMSDLSTSSKSEKASTSTRRSLTEDLDEDDVAEYDDEEEDEELPRKMKVLNDKNKSTSIKQEKLNELPSPLSSDFSDVDEEKSTLTHLKLQQQQQQPVDNYVSTPLSLPEDSVDFINPGNLKIESDENFLLSSNTLQIKHENDTDYITTAPSGSINDFFNSYDISESNRLHHPAVMTDSSLHITAGSIGFFSLIGGGESSVAGRRSSVGTYQLTCIAIR

SEQ ID NO: 73 - DNA, 2598 bp, synthetic construct
  misc_feature 1..2598: note = Synthetic polynucleotide
  source 1..2598: mol_type = other DNA
atgttggggggaattataggtggtctgacaggaaacaaaaatgcaagattgaagggttcactagttttgatgagaaagaatgctttggatattaacgacttcggcgctaccgtgattgaacgtattagcgaatttcttggtagaggagttacgtgtcaattagtctcaagctccttagtagacccaaacaacggtaataggggtagagttgggactgaggcttccctagagcagtggttgacatctctaccttctttgacaacgggtgaatccaagttcggtgtaacgtttgaatgggaagtcgaaaagatggggattcctggtgcgatcatcgttaaaaataaccacgctgccgagttcttcctgaaaacgatcacactagacaacgtccctggacacggggcagttgtgtttgtagccaacagctggatctaccctgcgtctaaataccgttacaatagagttttcttttccaacgacacaagtttgccttcaaagatggccgcagcgttaaagccatatagagacgatgaactaagaaaccttagaggtgatgatcaacaaggtccgtatcaggaacatgatagagtgtacaggtatgatgtctacaacgacttgggtgaacctgactctggaaatccgagaccagtcttaggagggtcacctgatcgtccttaccctagacgtggtagaacaggtagaaagcctactaaaactgatcctactgccgaatctagactgtctttgttagaaaatatctacgtcccacgtgacgaaaggtttggccaccttaagatggccgattttttgggatactcaatcaaagccttagtggatggaatagttcctgcaatacgtacctatgtggatttgactccaggtgagtttgacagcttcaaggacatcttgaagttgtatgaaggtggtttaaagctgccatctatacctgcacttgaggaattaaggaagagattccctttgcagttggtcaaggatctaatacctgggggagattatctgctgaaacttcctatgccgcatgtaatcagagaggataagaaagcctggatgacggatgatgagtttgccagggaaatcttggctggtgttaatccgatggttatcgcaagattaacggaatttccgccagagccattagacccagcgagatacggtgatcaaacatcaactatcaccgcagctcatgttgaaagaggtttggaaggtttgactgtccagcaagcaattgatgggaaccttctatacgtcgtcgaccaccatgaccacttcatgccttatcttctggatattaactcactggacgataattttatctacgccacaagaacacttttgttccttagaggagacggtacacttgctcttgccatcgaattgtctttgcctcacttacaggatgatggcctgatcactgccagaagcaccgtttacacgccagctgccgccgcagcacctgctcctgcgctggaatggtgggtttggcaattagctaaggcgtacgtaaacgtaaacgattactgctggcaccagctgatcagtcactggttaaatacccatgcagtcatggagccatttgtcattgcgaccaatcgtcaactgtcagtggctcatccagtccataagctgttattgcctcattatagggacacaatgaccattaatggtttagctagacaaacccttattaacggtggtggcatattcgagatgacagtttttcctagaaacgacgctttggcaatgtcatctgccttttacaaggattggagcttcgccgaccaggccttaccagacgacttggttaagagaggtgttagaacagaccctgcttcaccttataaggtccgtctgcttatcgaggattacccttacgcgaacgacggattagctgtatgcacgcctattgagcaatgggctactgaataccttgccatttactacccaaacgatggtgtcttacaaggtgatgctgaattacaggcctggtggaaagaagtcagagaagtcgggcatggggacatcaaagacgctacgtggtggcctgagatgaaaacagtcgcagatgttgtgaaagcttgtgctactatcatctggataggttcagctctgcatgctgcagtaaacttcggccaatacccttatgcaggatatttgccaaatagaccaagcgtgagtagaagggctatgcctgaacctggaaccaaagaatacgatgaacttgcgagagatcccgaaaaagtattcgttagaaccatcactaaacagatgcaagccatcgttgggatttctttgttagaaatacttagcaaacactcttctgacgaggtttacttaggtcagagggacactcctgaatggacatctgatgccaaagccttggaggcgtttaagaggttcggtgctagactgaccgagatagaatcccgtgtcgttgcaatgaacaaagatccacacagaaagaatagagtcggcccaactaacttcccttacaccctgttgtatccgaatacatcagacctgaagggagatgctgccggactttcagctagagggattcctaattccatatcaatttaa

SEQ ID NO: 74 - protein, 865 aa, Oryza sativa
MLGGIIGGLTGNKNARLKGSLVLMRKNALDINDFGATVIERISEFLGRGVTCQLVSSSLVDPNNGNRGRVGTEASLEQWLTSLPSLTTGESKFGVTFEWEVEKMGIPGAIIVKNNHAAEFFLKTITLDNVPGHGAVVFVANSWIYPASKYRYNRVFFSNDTSLPSKMAAALKPYRDDELRNLRGDDQQGPYQEHDRVYRYDVYNDLGEPDSGNPRPVLGGSPDRPYPRRGRTGRKPTKTDPTAESRLSLLENIYVPRDERFGHLKMADFLGYSIKALVDGIVPAIRTYVDLTPGEFDSFKDILKLYEGGLKLPSIPALEELRKRFPLQLVKDLIPGGDYLLKLPMPHVIREDKKAWMTDDEFAREILAGVNPMVIARLTEFPPEPLDPARYGDQTSTITAAHVERGLEGLTVQQAIDGNLLYVVDHHDHFMPYLLDINSLDDNFIYATRTLLFLRGDGTLALAIELSLPHLQDDGLITARSTVYTPAAAAAPAPALEWWVWQLAKAYVNVNDYCWHQLISHWLNTHAVMEPFVIATNRQLSVAHPVHKLLLPHYRDTMTINGLARQTLINGGGIFEMTVFPRNDALAMSSAFYKDWSFADQALPDDLVKRGVRTDPASPYKVRLLIEDYPYANDGLAVCTPIEQWATEYLAIYYPNDGVLQGDAELQAWWKEVREVGHGDIKDATWWPEMKTVADVVKACATIIWIGSALHAAVNFGQYPYAGYLPNRPSVSRRAMPEPGTKEYDELARDPEKVFVRTITKQMQAIVGISLLEILSKHSSDEVYLGQRDTPEWTSDAKALEAFKRFGARLTEIESRVVAMNKDPHRKNRVGPTNFPYTLLYPNTSDLKGDAAGLSARGIPNSISI

SEQ ID NO: 75 - DNA, 1848 bp, synthetic construct
  misc_feature 1..1848: note = Synthetic polynucleotide
  source 1..1848: mol_type = other DNA
atgcattgtatggcggttagacacttcgctccttcttccagtctttccatctttagcagcaccaacattaacaaccatttcttcggaagagagatctttacgcctaaaacttctaacatcactaccaaaaagagcagatctcgtcctaattgtaatccgatccagtgcagccttgcaaagtcaccatcctcagatacgtcaactatagtccgtaggtcagctaactacgacccacctatatggtccttcgatttcatccaaagcttaccgtgtaagtacaagggtgaaccatacacaagcagatctaacaagttgaaagaagaagtgaagaagatgcttgtaggtatggaaaacagcttggtgcagttggaattgatcgacaccttgcagagattgggcatttcataccacttcgagaatgaaattatcagcatcctgaaagaatatttcacaaatatctcaacgaacaaaaacccgaaatacgacttatacgcaactgcgttggaatttagactgctgagggaatacggatacgcaatcccacaggagatctttaacgactttaaggatgagactgggaagttcaaagctagtatcaagaacgacgacataaagggagtgctggcgctatatgaggctagcttctacgtcaaaaatggcgaaaatatccttgaagaagccagagtctttactacggaatacttgaagagatatgtgatgatgatcgaccagaacattatattgaacgacaacatggcaatcctagtgagacatgccttggagatgccacttcattggagaacgattagagcagaagccaagtggttcattgaggaatacgaaaaaacgcaggacaagaacggtaccttgttggaatttgccaaattggacttcaatatgctgcaatccatatttcaagaggacctaaagcatgtcagcagatggtgggaacactcagaactaggcaagaacaagatggtttatgcgcgtgatagactggttgaagcattcctatggcaagttggcgtcagatttgagcctcaattctcacattttaggagaatatctgctagaatatatgctcttatcacgataatcgacgatatctacgatgtttatggtactttggaagagcttgagttatttactaaggcggtcgagaggtgggatgctaaaactatccacgagttaccagactacatgaagctgccattctttacattgttcaacacagtgaacgagatggcttacgatgtgttggaagagcacaattttgttacagtggaatatctgaagaactcatgggcggaactatgtagatgctacttagaagaagcaaagtggttctattccggctataagcctacgttgaagaagtacattgagaatgcatctttaagcattggcgggcagatcatcttcgtgtatgcctttttctctctgacaaaatctattactaatgaggcgttagaatcacttcaggaaggtcatcatgctgcgtgtagacaaggttcattgatgttgagacttgcagatgatttgggcactctgtctgacgaaatgaaacgtggagatgtgccgaaatctatacagtgctatatgcatgatacaggagctagcgaagacgaagcccgtgaacatattaagtttctgatctccgaaatctggaaggaaatgaacgatgaagacgaatacaactcaattttttccaaggaatttgttcaagcatgtaaaaacttgggcagaatgtctttatttatgtatcaacatggtgatggacacgcttctcaggactcacattccaggaaaagaatatccgatcttataattaaccccatacctctgtaa

SEQ ID NO: 76 - protein, 615 aa, Cannabis sativa
MHCMAVRHFAPSSSLSIFSSTNINNHFFGREIFTPKTSNITTKKSRSRPNCNPIQCSLAKSPSSDTSTIVRRSANYDPPIWSFDFIQSLPCKYKGEPYTSRSNKLKEEVKKMLVGMENSLVQLELIDTLQRLGISYHFENEIISILKEYFTNISTNKNPKYDLYATALEFRLLREYGYAIPQEIFNDFKDETGKFKASIKNDDIKGVLALYEASFYVKNGENILEEARVFTTEYLKRYVMMIDQNIILNDNMAILVRHALEMPLHWRTIRAEAKWFIEEYEKTQDKNGTLLEFAKLDFNMLQSIFQEDLKHVSRWWEHSELGKNKMVYARDRLVEAFLWQVGVRFEPQFSHFRRISARIYALITIIDDIYDVYGTLEELELFTKAVERWDAKTIHELPDYMKLPFFTLFNTVNEMAYDVLEEHNFVTVEYLKNSWAELCRCYLEEAKWFYSGYKPTLKKYIENASLSIGGQIIFVYAFFSLTKSITNEALESLQEGHHAACRQGSLMLRLADDLGTLSDEMKRGDVPKSIQCYMHDTGASEDEAREHIKFLISEIWKEMNDEDEYNSIFSKEFVQACKNLGRMSLFMYQHGDGHASQDSHSRKRISDLIINPIPL

SEQ ID NO: 77 - DNA, 6702 bp, Saccharomyces cerevisiae
  source 1..6702: mol_type = genomic DNA
atgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactatctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgtttctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataa

SEQ ID NO: 78 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 79 - DNA, 6702 bp, synthetic construct
  misc_feature 1..6702: note = Synthetic polynucleotide
  source 1..6702: mol_type = other DNA
atgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactagctgatggtggtcttttgattgccataggcggtaaatcgcataccatctattggaaagaagaagttgctgctacaagattagccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgttgctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataa

SEQ ID NO: 80 - protein, 2153 aa, synthetic construct
  REGION 1..2153: note = Synthetic polypeptide
WAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLADGGLLIAIGGKSHTIYWKEEVAATRLAVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVAVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 81 - DNA, 55 bp, synthetic construct
  misc_feature 1..55: note = Synthetic polynucleotide
  source 1..55: mol_type = other DNA
gtttcgaataaacacacataaacaaacaaaatgagcgaagaaagcttattcgagt

SEQ ID NO: 82 - DNA, 28 bp, synthetic construct
  misc_feature 1..28: note = Synthetic polynucleotide
  source 1..28: mol_type = other DNA
caatcaaaagaccaccatcagctagttg

SEQ ID NO: 83 - DNA, 29 bp, synthetic construct
  misc_feature 1..29: note = Synthetic polynucleotide
  source 1..29: mol_type = other DNA
gtgatatcatactgcgtcaactagctgat

SEQ ID NO: 84 - DNA, 30 bp, synthetic construct
  misc_feature 1..30: note = Synthetic polynucleotide
  source 1..30: mol_type = other DNA
acaaagtagtcatagagtcaacggctaatc

SEQ ID NO: 85 - DNA, 29 bp, synthetic construct
  misc_feature 1..29: note = Synthetic polynucleotide
  source 1..29: mol_type = other DNA
gctacaagattagccgttgactctatgac

SEQ ID NO: 86 - DNA, 25 bp, synthetic construct
  misc_feature 1..25: note = Synthetic polynucleotide
  source 1..25: mol_type = other DNA
aaatctgaaacagcaacagccctgt

SEQ ID NO: 87 - DNA, 25 bp, synthetic construct
  misc_feature 1..25: note = Synthetic polynucleotide
  source 1..25: mol_type = other DNA
tatgaacagggctgttgctgtttca

SEQ ID NO: 88 - DNA, 60 bp, synthetic construct
  misc_feature 1..60: note = Synthetic polynucleotide
  source 1..60: mol_type = other DNA
agaatatttaatttatgtatgtaagttattatttcaaagtcttcaacaatttttctttat

SEQ ID NO: 89 - DNA, 60 bp, synthetic construct
  misc_feature 1..60: note = Synthetic polynucleotide
  source 1..60: mol_type = other DNA
tttgtttgtttatgtgtgtttattcgaaactaagttcttggtgttttaaaactaaaaaaa

SEQ ID NO: 90 - DNA, 200 bp, synthetic construct
  misc_feature 1..200: note = Synthetic polynucleotide
  source 1..200: mol_type = other DNA
tacccccaaaatttcttctctaaacgatgtgcttggcctttgttttggtttttgatgtcggtcgtttgaggccccttgcggaaaatcgagatcgccgaatggcacgcgagggaagggaaataaggtttaaaggcactgaaacaataggcaagaagtaggcgagagccgacatacgagagaataaaaaacacgctttttca

SEQ ID NO: 91 - DNA, 60 bp, synthetic construct
  misc_feature 1..60: note = Synthetic polynucleotide
  source 1..60: mol_type = other DNA
cttacatacataaattaaatattctgtctttttgttatatcccgaccatgccatcacttt

SEQ ID NO: 92 - DNA, 200 bp, synthetic construct
  misc_feature 1..200: note = Synthetic polynucleotide
  source 1..200: mol_type = other DNA
aacctcgttaaattttcataaagtgcatatcgtgtaatgtaaaaagtaaaaagataaaaaagactcatatgaaatcttgctgcatggagacaagtgcactcgaattctttacttatattttcgctcttgagctcgtccttttactagcatatcaatatccgtttcattgaaaagtggtaatttagatagataacgagata

SEQ ID NO: 93 - DNA, 24 bp, synthetic construct
  misc_feature 1..24: note = Synthetic polynucleotide
  source 1..24: mol_type = other DNA
tctaaacgatgtgcttggcctttg

SEQ ID NO: 94 - DNA, 33 bp, synthetic construct
  misc_feature 1..33: note = Synthetic polynucleotide
  source 1..33: mol_type = other DNA
ataaagtgcatatcgtgtaatgtaaaaagtaaa

SEQ ID NO: 95 - DNA, 1638 bp, Cannabis sativa
  source 1..1638: mol_type = genomic DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 96 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 97 - DNA, 1557 bp, Cannabis sativa
  source 1..1557: mol_type = genomic DNA
atgaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 98 - protein, 518 aa, Cannabis sativa
MNPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

(19) United States
(12) Patent Application Publication
(10) Pub. No.: US 2023/0279449 A1
(43) Pub. Date: Sep. 7, 2023

(54) COMPOSITIONS AND METHODS FOR ENHANCING RECOMBINANT BIOSYNTHESIS OF CANNABINOIDS

(71) Applicant: Willow Biosciences, Inc., Calgary (CA)

(72) Inventors: Xueyang FENG, Fremont, CA (US); Trish CHOUDHARY, Belmont, CA (US); Sheela MULEY, Fremont, CA (US); Vincent KAM, Sunnyvale, CA (US)

(73) Assignee: Willow Biosciences, Inc., Calgary (CA)

(21) Appl. No.: 18/049,603

(22) Filed: Oct. 25, 2022

Related U.S. Application Data
(63) Continuation of application No. PCT/US2021/029447, filed on Apr. 27, 2021.
(60) Provisional application No. 63/017,594, filed on Apr. 29, 2020.

(51) Int. Cl. C12P 7/22; C12N 15/52

(52) U.S. Cl. CPC C12P 7/22; C12N 15/52

(57) ABSTRACT

The present disclosure provides recombinant host cells comprising a pathway capable of producing a cannabinoid and a heterologous nucleic acid that encodes a protein not in the pathway that enhances the host cells’ ability to produce the cannabinoid. The disclosure also provides methods of using host cells to produce cannabinoids.

### CROSS-REFERENCE TO RELATED APPLICATIONS

This application is a continuation of International Application No. PCT/US2021/029447, filed Apr. 27, 2021, which claims priority benefit to U.S. Provisional Application No. 63/017,594 filed Apr. 29, 2020, each of which is hereby incorporated by reference herein.

### FIELD

The present disclosure relates generally to recombinant host cells comprising a cannabinoid biosynthesis pathway and a heterologous nucleic acid that encodes an enhancer protein not in the pathway that acts to improve the ability of the host cell to produce the cannabinoids, and methods for using the recombinant host cells for cannabinoid production.

### REFERENCE TO SEQUENCE LISTING

The official copy of the Sequence Listing is submitted concurrently with the specification via USPTO Patent Center as an WIPO Standard ST.26 formatted XML file with file name “13421-002WO1.xml”, a creation date of Oct. 25, 2022, and a size of 200,955 bytes. This Sequence Listing filed via USPTO Patent Center is part of the specification and is incorporated in its entirety by reference herein. This sequence listing corresponds to the ST.26 formatted version of the ST.25 formatted with a file name “13421-002WO1_SeqList-ST25.txt” that was filed with the parent application International Appl. No. PCT/US2021/029447 on Apr. 27, 2021.

### BACKGROUND

Cannabinoids are a class of compounds that act on endocannabinoid receptors and include the phytocannabinoids naturally produced by Cannabis sativa. Cannabinoids include Δ⁹-tetrahydrocannabinol (THC), cannabidiol (CBD) and more than 80 related metabolites and synthetically produced compounds. Cannabinoids are increasingly used to treat a range of diseases and conditions such as multiple sclerosis and chronic pain. Current large-scale production of cannabinoids for pharmaceutical or other use is through extraction from plants. These plant-based production processes, however, have several challenges including susceptibility of the plants to inconsistent production caused by variance in biotic and abiotic factors, difficulty reproducing identical cannabinoid accumulation profiles, and difficulty in producing a single cannabinoid compound with purity high enough for pharmaceutical applications. While some cannabinoids can be produced as a single pure product via chemical synthesis, these processes have proven very costly and too costly for large-scale production.

More economical biosynthetic approaches to cannabinoid production are being developed using microbial hosts. These processes have the potential to be robust, scalable, and capable of producing single cannabinoid compound with higher purity compared to other current processes. Several biosynthetic systems for cannabinoid compound have been reported (see e.g., WO2019071000, WO2018200888, WO2018148849, WO2019014490, US20180073043, US20180334692, and WO2019046941). However, these biosynthetic systems still are not efficient in the biosynthesis of cannabinoids. The possible reasons for the low-yield conversion of these systems include poor protein expression, stressed cell growth, suboptimal metabolite transport, and lack of accessory genes required for optimal cannabinoid biosynthesis.

There exists a need for improved methods for the production of cannabinoid compounds. In particular, there is a need to improve the performance of microbial hosts in carrying out the recombinant biosynthesis of cannabinoid compounds.

### SUMMARY

The present disclosure relates generally to recombinant host cells comprising a pathway capable of producing a cannabinoid and a heterologous nucleic acid that encodes an off-pathway protein that enhances the host cells’ ability to produce the cannabinoids. The disclosure also relates to methods for using the recombinant host cells for cannabinoid production, and compositions comprising the heterologous nucleic acids. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides recombinant host cell comprising a pathway capable of producing a cannabinoid and a heterologous nucleic acid that encodes a protein not in the pathway.

In at least one embodiment of the recombinant host cell, production of the cannabinoid is increased at least 1.5-fold, at least 2-fold, at least 3-fold, at least 4-fold, at least 5-fold, or more, relative to a control recombinant host cell comprising the pathway and without the heterologous nucleic acid.

In at least one embodiment of the recombinant host cell, the encoded protein is of a type selected from: a chaperonin, an efflux pump protein, a fatty acid pathway protein, a lipid transfer protein, a mevalonate pathway protein, a methylerythritol 4-phosphate pathway protein, a permease, and a terpenoid biosynthesis pathway protein.

In at least one embodiment of the recombinant host cell, the encoded protein is derived from a source selected from: Arabidopsis thaliana, Bacillus subtilis, Bifidobacterium longum, Cannabis sativa, Corynebacterium glutamicum, Escherichia coli, Homo sapiens, Komagataella pastoris, Komagataella phaffii, Neisseria, Oryza sativa, Papaver somniferum, Picrorhiza kurrooa, Pseudomonas aeruginosa, Ricinus communis, Saccharomyces cerevisiae, Streptomyces cyanogenus, Streptomyces peucetius, and Vibrio cholerae.

In at least one embodiment of the recombinant host cell, the encoded protein encoded is derived from a source that is not Cannabis sativa.

In at least one embodiment of the recombinant host cell, the encoded protein is selected from: 1-deoxy-D-xylulose 5-phosphate reductoisomerase, chloroplastic [Ricinus communis]; 4-(cytidine 5′-phospho)-2-C-methyl-D-erithritol kinase [Arabidopsis thaliana]; 4-hydroxy-3-methylbut-2-enyl diphosphate reductase, chloroplastic [Ricinus communis]; 4-hydroxy-3-methylbut-2-enyl diphosphate synthase [Arabidopsis thaliana]; ABC transporter ATP-binding protein [Bifidobacterium longum]; Acetoacetyl-CoA thiolase [Picrorhiza kurrooa]; Acetyl-CoA C-acetyltransferase [Saccharomyces cerevisiae S288C]; ATP-binding cassette domain-containing protein [Streptomyces peucetius]; bZIP transcription factor (ATF/CREB1 homolog) that regulates the unfolded protein response [Komagataella phaffii GS115]; Chaperone of the endoplasmic reticulum lumen [Komagataella phaffii CBS 7435]; Chaperonin-containing T-complex subunit CCT4 [Saccharomyces cerevisiae S288C]; Chaperonin-containing T-complex subunit CCT5 [Saccharomyces cerevisiae S288C]; Extended synaptotagmin-2 [Homo sapiens]; Hsp90 family chaperone HSP82 [Saccharomyces cerevisiae S288C]; LanJ [Streptomyces cyanogenus]; Lipid A ABC transporter ATP-binding protein/permease MsbA [Escherichia coli]; Lipoxygenase [Oryza sativa Japonica Group]; Multidrug efflux MFS transporter Cmr [Corynebacterium glutamicum]; MULTISPECIES: multidrug efflux MATE transporter NorM [Neisseria]; Nsg2p [Saccharomyces cerevisiae S288C]; Pleckstrin homology domain-containing family A member 8 isoform 1 [Homo sapiens]; PRELI domain-containing protein 1, mitochondrial isoform 1 precursor [Homo sapiens]; PRELI domain-containing protein 2 isoform a [Homo sapiens]; Protein disulphide isomerase [Komagataella pastoris]; Purine permease 1-like [Papaver somniferum]; Purine permease 1-like [Papaver somniferum]; Small multidrug resistance efflux transporter EbrA [Bacillus subtilis subsp. subtilis str. BSP1]; Terpene synthase [Cannabis sativa]; Thiol oxidase required for oxidative protein folding in the endoplasmic reticulum [Komagataella phaffii GS115]; Type I secretion system permease/ATPase [Pseudomonas aeruginosa]; and VceA [Vibrio cholerae].

In at least one embodiment of the recombinant host cell, the encoded protein comprises an amino acid sequence having at least 90% identity to any one of even-numbered SEQ ID NOs: 16-80.

In at least one embodiment of the recombinant host cell, the heterologous nucleic acid comprises a nucleotide sequence having at least 90% identity to any one of odd-numbered SEQ ID NOs: 15-79.

In at least one embodiment of the recombinant host cell, the pathway comprises enzymes capable of converting hexanoic acid to CBGA.

In at least one embodiment of the recombinant host cell, the pathway comprises at least the enzyme PT4.

In at least one embodiment of the recombinant host cell, the pathway comprises enzymes capable of catalyzing reactions (i) - (iv):

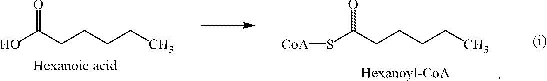

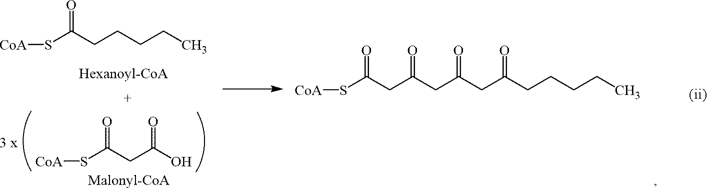

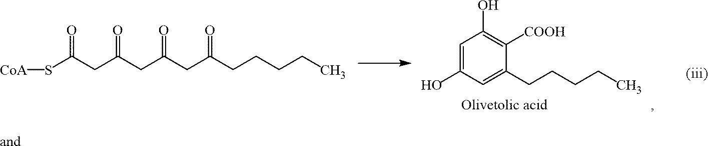

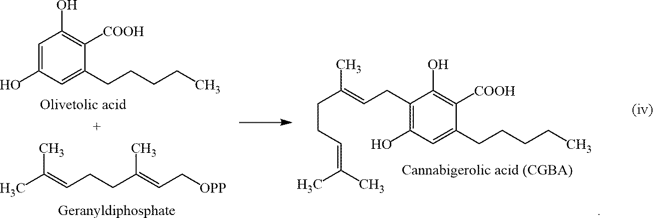

In at least one embodiment of the recombinant host cell, the pathway comprises at least the enzymes: AAE, OLS, OAC, and PT4; optionally, wherein the enzymes AAE, OLS, OAC, and PT4 have an amino acid sequence of at least 90% identity to SEQ ID NO: 2 (AAE), SEQ ID NO: 4 (OLS), SEQ ID NO: 6 (OAC), and SEQ ID NO: 8 or 10 (PT4), respectively.

In at least one embodiment of the recombinant host cell, the pathway comprises enzymes capable of converting hexanoic acid to CBGA and further comprises an enzyme capable of catalyzing the conversion of CBGA to Δ⁹-THCA, CBDA, and/or CBCA. In at least one embodiment, the pathway is capable of catalyzing the conversion of CBGA to Δ⁹-THCA, CBDA, and/or CBCA and further comprises: THCA synthase, CBDA synthase, and/or CBCA synthase. In at least one embodiment, the pathway comprises: (i) a CBDA synthase having an amino acid sequence of at least 90% identity to SEQ ID NO: 12 or 14; or (ii) a THCA synthase having an amino acid sequence of at least 90% identity to SEQ ID NO: 96 or 98..

In at least one embodiment of the recombinant host cell, the cannabinoid produced by the host cell is selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

In at least one embodiment, the present disclosure also provides methods of using the recombinant host cell of the disclosure for producing a cannabinoid, the methods comprising: (a) culturing in a suitable medium a recombinant host cell of the present disclosure (e.g., a host cell comprising a pathway capable of producing a cannabinoid and a heterologous nucleic acid that encodes a protein not in the pathway); and (b) recovering the produced cannabinoid. In at least one embodiment, the method further comprises contacting a cell-free extract of the culture with a biocatalytic reagent or chemical reagent.

In at least one embodiment of the method for producing a cannabinoid, the encoded protein is derived from a source selected from: Arabidopsis thaliana, Bacillus subtilis, Bifidobacterium longum, Cannabis sativa, Corynebacterium glutamicum, Escherichia coli, Homo sapiens, Komagataella pastoris, Komagataella phaffii, Neisseria, Oryza sativa, Papaver somniferum, Picrorhiza kurrooa, Pseudomonas aeruginosa, Ricinus communis, Saccharomyces cerevisiae, Streptomyces cyanogenus, Streptomyces peucetius, and Vibrio cholerae.

In at least one embodiment of the method for producing a cannabinoid, the encoded protein encoded is derived from a source that is not Cannabis sativa.

In at least one embodiment of the method for producing a cannabinoid, the encoded protein is selected from: 1-deoxy-D-xylulose 5-phosphate reductoisomerase, chloroplastic [Ricinus communis]; 4-(cytidine 5′-phospho)-2-C-methyl-D-erithritol kinase [Arabidopsis thaliana]; 4-hydroxy-3-methylbut-2-enyl diphosphate reductase, chloroplastic [Ricinus communis]; 4-hydroxy-3-methylbut-2-enyl diphosphate synthase [Arabidopsis thaliana]; ABC transporter ATP-binding protein [Bifidobacterium longum]; Acetoacetyl-CoA thiolase [Picrorhiza kurrooa]; Acetyl-CoA C-acetyltransferase [Saccharomyces cerevisiae S288C]; ATP-binding cassette domain-containing protein [Streptomyces peucetius]; bZIP transcription factor (ATF/CREB1 homolog) that regulates the unfolded protein response [Komagataella phaffii GS115]; Chaperone of the endoplasmic reticulum lumen [Komagataella phaffii CBS 7435]; Chaperonin-containing T-complex subunit CCT4 [Saccharomyces cerevisiae S288C]; Chaperonin-containing T-complex subunit CCT5 [Saccharomyces cerevisiae S288C]; Extended synaptotagmin-2 [Homo sapiens]; Hsp90 family chaperone HSP82 [Saccharomyces cerevisiae S288C]; LanJ [Streptomyces cyanogenus]; Lipid A ABC transporter ATP-binding protein/permease MsbA [Escherichia coli]; Lipoxygenase [Oryza sativa Japonica Group]; Multidrug efflux MFS transporter Cmr [Corynebacterium glutamicum]; MULTISPECIES: multidrug efflux MATE transporter NorM [Neisseria]; Nsg2p [Saccharomyces cerevisiae S288C]; Pleckstrin homology domain-containing family A member 8 isoform 1 [Homo sapiens]; PRELI domain-containing protein 1, mitochondrial isoform 1 precursor [Homo sapiens]; PRELI domain-containing protein 2 isoform a [Homo sapiens]; Protein disulphide isomerase [Komagataella pastoris]; Purine permease 1-like [Papaver somniferum]; Purine permease 1-like [Papaver somniferum]; Small multidrug resistance efflux transporter EbrA [Bacillus subtilis subsp. subtilis str. BSP1]; Terpene synthase [Cannabis sativa]; Thiol oxidase required for oxidative protein folding in the endoplasmic reticulum [Komagataella phaffii GS115]; Type I secretion system permease/ATPase [Pseudomonas aeruginosa]; and VceA [Vibrio cholerae].

In at least one embodiment of the method for producing a cannabinoid, the encoded protein comprises an amino acid sequence having at least 90% identity to any one of even-numbered SEQ ID NOs: 16-80.

In at least one embodiment of the method for producing a cannabinoid, the heterologous nucleic acid comprises a nucleotide sequence having at least 90% identity to any one of odd-numbered SEQ ID NOs: 15-79.

In at least one embodiment, the present disclosure also provides method for making a recombinant host cell for producing a cannabinoid comprising introducing into a host cell: (a) a first set of nucleic acids that encode a pathway capable of producing a cannabinoid; and (b) a heterologous nucleic acid that encodes a protein not in the pathway.

In at least one embodiment of the method for making the recombinant host cell, the encoded protein is of a type selected from: a chaperonin, an efflux pump protein, a fatty acid pathway protein, a lipid transfer protein, a mevalonate pathway protein, a methylerythritol 4-phosphate pathway protein, a permease, and a terpenoid biosynthesis pathway protein.

In at least one embodiment of the method for making the recombinant host cell, the encoded protein comprises an amino acid sequence having at least 90% identity to any one of even-numbered SEQ ID NOs: 16-80. In at least one embodiment, the heterologous nucleic acid comprises a nucleotide sequence having at least 90% identity to any one of odd-numbered SEQ ID NOs: 15-79.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts an exemplary pathway capable of converting hexanoic acid to CBGA.

FIG. 2 depicts an exemplary pathway capable of catalyzing the conversion of CBGA to Δ⁹-THCA, CBDA, and/or CBCA.

FIG. 3 depicts a yeast expression vector with auxotrophic marker (LEU2 gene) and constitutive TDH3 promoter into which heterologous nucleic acids encoding proteins providing enhanced cannabinoid biosynthesis were sub-cloned for screening as described in Example 1.

FIG. 4 depicts a schematic representation of the Acc1 and Acc1_3p expression cassettes used to target the X-3 locus of the Saccharomyces cerevisiae genomic DNA as described in Example 2.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in Sambrook et al., Molecular Cloning-A Laboratory Manual (2nd Ed.), Vols. 1-3, Cold Spring Harbor Laboratory, Cold Spring Harbor, N.Y., 1989 (hereinafter “Sambrook”); Current Protocols in Molecular Biology, F. M. Ausubel et al., eds., Current Protocols, a joint venture between Greene Publishing Associates, Inc. and John Wiley & Sons, Inc. (supplemented through 2011) (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Definitions

“Cannabinoid” refers to a compound that acts on cannabinoid receptor, and is intended to include the endocannabinoid compounds that are produced naturally in animals, the phytocannabinoid compounds produced naturally in cannabis plants, and the synthetic cannabinoids compounds. Exemplary cannabinoids of the present disclosure include those compounds listed in Table 3 (below).

“Pathway” refers an ordered sequence of enzymes that act in a linked series to convert an initial substrate molecule into final product molecule. As used herein, “pathway” is intended to encompass naturally-occurring pathways and non-naturally occurring, recombinant pathways. Accordingly, a pathway of the present disclosure can include a series of enzymes that are naturally-occurring and/or non-naturally occurring, and can include a series of enzymes that act in vivo or in vitro.

“Pathway capable of producing a cannabinoid” refers to a pathway that can convert an initial substrate molecule, such as hexanoic acid, into a final product molecule that is a cannabinoid, such as cannabigerolic acid (CBGA). For example, the four enzymes AAE, OLS, OAC, and PT4 which convert hexanoic acid to CBGA, form a pathway capable of producing a cannabinoid.

“Conversion” as used herein refers to the enzymatic conversion of the substrate(s) to the corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Substrate” as used herein in the context of an enzyme mediated process refers to the compound or molecule acted on by the enzyme.

“Product” as used herein in the context of an enzyme mediated process refers to the compound or molecule resulting from the activity of the enzyme.

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism’s genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; McInerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length, but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95 % sequence identity, or at least 99% sequence identity, as compared to a reference sequence over a comparison window of at least 20 nucleoside or amino acid residue positions, frequently over a window of at least 30-50 positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition), and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

### Recombinant Host Cells With Enhanced Cannabinoid Production

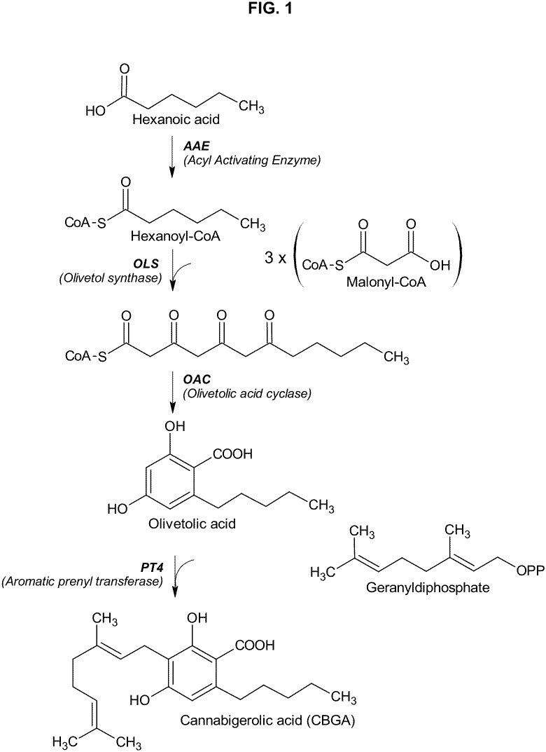

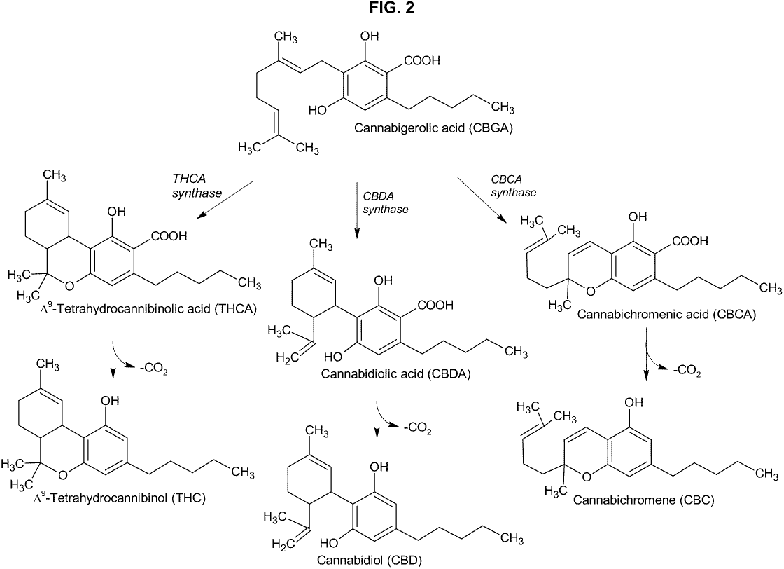

The present disclosure provides recombinant host cells (e.g., S. cerevisiae) already transformed with a cannabinoid biosynthesis pathway, which further comprise a heterologous nucleic acid that encodes a protein that is not a part of the enzymatic pathway to the cannabinoid product. For example, the additional heterologous nucleic acid does not encode an enzyme that acts on any of the substrate, intermediate, and/or product compounds in the cannabinoid biosynthesis pathway already present in the recombinant host cell. In the context of the recombinant host cells already transformed with a cannabinoid biosynthesis pathway, an exemplary cannabinoid biosynthesis pathway is one capable of converting hexanoic acid (HA) cannabigerolic acid (CBGA) as depicted in FIG. 1. The biosynthetic conversion of HA to CBGA is carried out by the sequence of enzymes, Acyl Activating Enzyme (AAE), Olivetol Synthase (OLS), Olivetolic Acid Cyclase (OAC), and a prenyltransferase (PT4). Although FIG. 1 depicts a four enzyme cannabinoid pathway from HA to CBGA, it is contemplated that shorter pathways comprising only the three enzyme, AAE, OLS, and OAC, could be incorporated into a host cell for the biosynthetic production of the cannabinoid precursor olivetolic acid (OA) from HA, or a pathway of PT4 and a cannabinoid synthase could be incorporated in a host cell for biosynthetic production of a cannabinoid from OA. As shown in FIG. 2, an extension of the four enzyme exemplary pathway of FIG. 1 with a cannabinoid synthase (e.g., CBDAS, THCAS, and/or CBCAS) allows for the biosynthetic production of one or more of the cannabinoids, Δ⁹-THCA (or “THCA”), CBDA, and/or CBCA. These cannabinoids are capable of further conversion by decarboxylation to provide the cannabinoids, Δ⁹-THC (or “THC”), CBD, and/or CBC. It is contemplated, that in some embodiments this further decarboxylation reaction can be carried out under in vitro reaction conditions using the cannabinoid acids separated and/or isolated from the recombinant host cells.

The presence of this additional heterologous nucleic acid and/or the “off-pathway” protein that it encodes, however, results in a recombinant host cell that produces the cannabinoid product in substantially enhanced yields (e.g., 1.5-fold or more increased) relative to a recombinant host cell comprising the same cannabinoid biosynthesis pathway but without the heterologous nucleic acid. As disclosed elsewhere herein, this enhancement of cannabinoid production is provided by heterologous nucleic acids encoding a protein of a type selected from: a chaperonin, an efflux pump protein, a fatty acid pathway protein, a lipid transfer protein, a mevalonate pathway protein, a methylerythritol 4-phosphate pathway protein, a permease, and a terpenoid biosynthesis pathway protein.

The unexpected and surprising technical effect of improved cannabinoid biosynthesis associated with the introduction of one or more heterologous nucleic acids that encode the expression of such “enhancer” or “helper” proteins in a recombinant host cell already capable of biosynthesizing a cannabinoid provides for an improved recombinant host cell, and associated improved methods of cannabinoid production. Accordingly, in at least one embodiment, the present disclosure provides a recombinant host cell comprising a pathway capable of producing a cannabinoid and a heterologous nucleic acid that encodes an enhancer protein that is not an enzyme in the pathway. As described further below, an exemplary pathway capable of biosynthesizing a cannabinoid in a recombinant host cell (e.g., yeast) can include four enzymes that convert hexanoic acid to cannabigerolic acid (CBGA).

The enhancer proteins provided by the present disclosure are not limited to proteins that are derived from Cannabis or from the host cell (e.g., Saccharomyces cerevisiae). Indeed, in at least one embodiment, the enhancer protein encoded by the heterologous nucleic acid is derived from a source selected from: Arabidopsis thaliana, Bacillus subtilis, Bifidobacterium longum, Cannabis sativa, Corynebacterium glutamicum, Escherichia coli, Homo sapiens, Komagataella pastoris, Komagataella phaffii, Neisseria, Oryza sativa, Papaver somniferum, Picrorhiza kurrooa, Pseudomonas aeruginosa, Ricinus communis, Saccharomyces cerevisiae, Streptomyces cyanogenus, Streptomyces peucetius, and Vibrio cholerae. In at least one embodiment of the recombinant host cells the encoded protein encoded is derived from a source that is not Cannabis sativa.

In at least one embodiment, the recombinant host cell comprising a heterologous nucleic acid that encodes such an enhancer protein is capable of producing the cannabinoid with a titer that is increased relative to a control recombinant host cell comprising the same cannabinoid biosynthesis pathway but without the heterologous nucleic acid. In at least one embodiment, the titer of cannabinoid produced is increased by at least 1.1-fold. 1.2-fold, 1.3-fold, 1.4-fold, 1.5-fold, 2-fold, 3-fold, 4-fold, 5-fold, 6-fold, 10-fold, or more relative to a control recombinant host cell that comprises the pathway without the heterologous nucleic acid encoding the enhancer protein.

Without intending to be bound by theory, it believed that the enhancer proteins encoded by the heterologous nucleic acids as described herein facilitate a range of metabolic mechanisms that help increase the output of a cannabinoid product from recombinant host cell engineered with a cannabinoid pathway. For example, it is believed that the following off-pathway functions of types of enhancer proteins can contribute to increased cannabinoid biosynthesis in recombinant host cell systems: (a) a chaperonins can provide favorable conditions for correct folding and prevent aggregation of proteins involved in cannabinoid synthesis in microbial hosts; (b) an efflux pump protein can assist the recombinant host in pumping out biosynthesized cannabinoids and thereby reduce their toxicity to the host; (c) a fatty acid pathway enzyme can help improve the biosynthesis of cannabinoid pathway precursor compounds in microbial hosts; (d) lipid transfer proteins (LTPs) can assist in the transfer of cannabinoid compounds from the cytoplasm to the cell membrane in microbial hosts; (e) methylerythritol 4-phosphate pathway (MEP) enzymes can provide an improved metabolic pathway for GPP biosynthesis that bypasses microbial host’s regulation of mevalonate pathway; (f) mevalonate pathway (MVA) enzymes can provide an improved MVA pathway that enhances GPP biosynthesis in microbial hosts; (g) permeases can potentially act to transport cannabinoids in microbial hosts; (h) protein folding proteins can provide increased levels of correctly folded recombinant proteins in microbial hosts; and (i) terpenoid biosynthesis pathway enzymes, such as terpenoid synthases, can potentially act as catalysts in the biosynthesis of cannabinoids in microbial hosts. As described further below (and in the Examples) specific enhancer proteins from each of the above-listed protein types and derived from variety of sources, have been identified through screening as capable of providing at least a 1.5-fold improvement of cannabinoid production in a recombinant host cell system.

As is described throughout the present disclosure, the recombinant host cells comprise a recombinant biosynthetic pathway for producing a cannabinoid. Generally, the pathway comprises a sequence of linked enzymes that convert a cannabinoid precursor substrate to a final cannabinoid compound product. As described elsewhere herein (e.g., Table 3), there are a wide range of cannabinoid products that are contemplated for such biosynthetic production in a recombinant host cell. In at least one embodiment, the recombinant pathway comprises at least an aromatic prenyl transferase (e.g., PT4), which catalyzes the final enzymatic conversion to produce the cannabinoid acid intermediate (e.g.,cannabigerolic acid (CBGA), cannabidivarinic acid (CBDVA), cannabinolic acid (CBNA), etc), that is a common intermediate in the biosynthesis of a wide range of cannabinoid products.

As described elsewhere herein, the recombinant cannabinoid biosynthesis pathway can comprise at least enzymes capable of converting hexanoic acid to cannabigerolic acid (CBGA). One such pathway capable of converting hexanoic acid to CBGA is illustrated in FIG. 1. Accordingly, in at least one embodiment of the recombinant host cell, the pathway capable of producing a cannabinoid comprises enzymes capable of catalyzing reactions (i) - (iv):

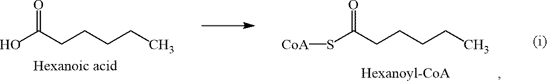

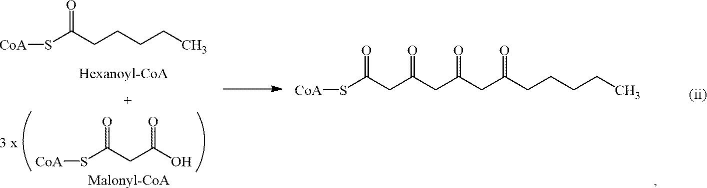

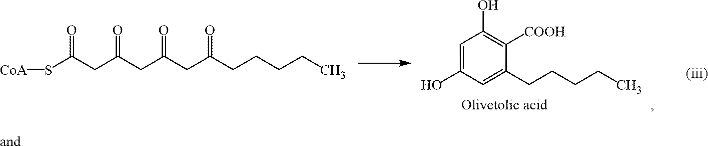

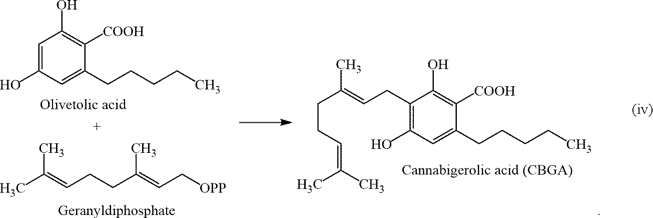

As shown in FIG. 1, exemplary enzymes capable of catalyzing reactions (i) - (iv) are: (i) acyl activating enzyme (AAE); (ii) olivetol synthase (OLS); (iii) olivetolic acid cyclase (OLA); and (iv) aromatic prenyl transferase (PT4).

As shown in FIG. 2, the cannabinoid compound, CBGA, that is produced by the pathway of FIG. 1, can be further converted to at least three other different cannabinoid compounds, Δ⁹-tetrahydrocannabinolic acid (THCA), cannabidiolic acid (CBDA), and/or cannabichromenic acid (CBCA). Accordingly, in at least one embodiment, the present disclosure provides a recombinant host cell comprising a pathway capable of converting hexanoic acid to CBGA and further comprising an enzyme capable of catalyzing the conversion of (v) CBGA to Δ⁹-THCA; (vi) CBGA to CBDA; and/or (vii) CBGA to CBCA. Thus, in at least one embodiment, the recombinant host cell comprises pathway capable of converting hexanoic acid to CBGA further comprises further comprises enzymes capable of catalyzing a reaction (v), (vi), and/or (vii):

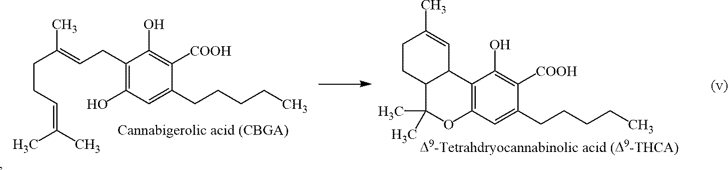

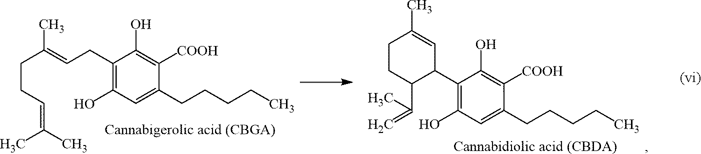

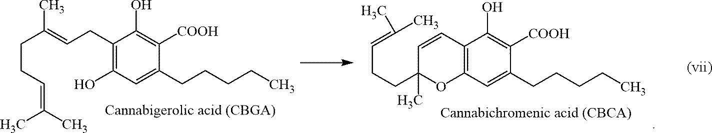

As shown in FIG. 2, exemplary enzymes capable of catalyzing reaction (v)-(vii) are: (v) THCA synthase (THCAS); (vi) CBDA synthase (CBDAS); and (vii) CBCA synthase (CBCAS).

Cannabinoid pathway enzymes that can be introduced into a recombinant host cell to provide the pathways illustrated in FIGS. 1 and 2 include, but are not limited to, the cannabinoid pathway enzymes from Cannabis sativa described in Table 1 (below).

TABLE 1

Cannabinoid pathway enzymes

| Enzyme Name (abbreviation) | GenBank Identifier | SEQ ID NO: (nt) | SEQ ID NO: (aa) |
|---|---|---|---|
| Acyl activating enzyme (AAE) | >AFD33345.1 acyl-activating enzyme 1 [Cannabis sativa] | 1 | 2 |
| Olivetol synthase (OLS) | >BAG14339.1 olivetol synthase [Cannabis sativa] | 3 | 4 |
| Olivetolic acid cyclase (OAC) | >AFN42527.1 olivetolic acid cyclase [Cannabis sativa] | 5 | 6 |
| Aromatic prenyltransferase (PT4) | > DAC76710.1 prenyltransferase 4, [Cannabis sativa] | 7 | 8 |
| CBDA synthase (CBDAS) | >BAF65033.1 cannabidiolic acid synthase [Cannabis sativa] | 11 | 12 |
| THCA synthase (THCAS) | >BAC41356.1 tetrahydrocannabinolic acid synthase [Cannabis sativa] | 95 | 96 |

In at least one embodiment, the recombinant host cell the pathway capable of producing a cannabinoid comprises at least the exemplary enzymes, wherein the enzymes have the amino acid sequences of SEQ ID NO: 2 (AAE), SEQ ID NO: 4 (OLS), SEQ ID NO: 6 (OAC), and SEQ ID NO: 8 (PT4). In at least one embodiment, the recombinant host cell the pathway capable of producing a cannabinoid further comprises: (i) the enzyme of SEQ ID NO: 12 (CBDAS), or (ii) the enzyme of SEQ ID NO: 96 (THCAS).

The exemplary cannabinoid pathway enzymes listed in Table 1 are the naturally occurring sequences from C. sativa, it also is contemplated, however, that cannabinoid pathway enzymes used in the recombinant host can include naturally occurring sequence homologs of these enzymes and/or enzymes having non-naturally occurring sequences. For example, enzymes with amino acid sequences engineered to function optimally in a particular enzyme pathway, and/or optimally for production of particular cannabinoid, and/or optimally in a particular host. Methods for preparing such non-naturally occurring enzyme sequences are known in the art and include methods for enzyme engineering such as directed evolution. In at least one embodiment, the amino acid sequence of non-naturally occurring enzyme can be modified at either its N- or C- terminus by truncation, or fusion. For example, in at least one embodiment of the pathway of producing a cannabinoid, the naturally occurring amino acid sequence of the PT4 enzyme of SEQ ID NO: 8 can be truncated at the N-terminus by up to 82 amino acids to provide the PT4 of SEQ ID NO: 10 (also referred to herein as “d82_PT4”), which is capable of functioning to produce the cannabinoid CBGA in a recombinant host cell. Accordingly, in at least one embodiment of the recombinant host cell, the pathway capable of producing a cannabinoid comprises at least enzymes having an amino acid sequence at least 90% identity to SEQ ID NO: 2 (AAE), SEQ ID NO: 4 (OLS), SEQ ID NO: 6 (OAC), and SEQ ID NOs: 10 (PT4). Similarly, it is contemplated that engineered versions of the AAE, OLS, OAC, and CBDAS enzymes can be prepared using methods known in the art, and used in the compositions and methods of the present disclosure. For example, the CBDAS enzyme of SEQ ID NO: 12 or the THCAS enzyme of SEQ ID NO: 96 can be truncated at the N-terminus by up to 28 amino acids to provide the d28_CBDAS enzyme of SEQ ID NO: 14 and the d28_THCAS enzyme of SEQ ID NO: 98. Accordingly, in at least one embodiment of the recombinant host cell, the pathway capable of producing a cannabinoid comprises at least enzymes having an amino acid sequence at least 90% identity to SEQ ID NO: 2 (AAE), SEQ ID NO: 4 (OLS), SEQ ID NO: 6 (OAC), SEQ ID NOs: 10 (d82_PT4), and SEQ ID NO: 14 (d28_CBDAS). Or in at least one embodiment of the recombinant host cell, the pathway capable of producing a cannabinoid comprises at least enzymes having an amino acid sequence at least 90% identity to SEQ ID NO: 2 (AAE), SEQ ID NO: 4 (OLS), SEQ ID NO: 6 (OAC), SEQ ID NOs: 10 (d82_PT4), and SEQ ID NO: 98 (d28_THCAS).

Other cannabinoid pathway enzymes useful in the recombinant host cells and associated methods of the present disclosure are known in the art, and can include naturally occurring enzymes obtained or derived from cannabis plants, (e.g., Cannabis sativa, and its sub-species, sativa, indica, and ruderalis), or non-naturally occurring enzymes that have been engineered based on the naturally occurring cannabis plant sequences. It is also contemplated that enzymes obtained or derived from other organisms (e.g., microorganisms) having a catalytic activity related to a desired conversion activity useful in a cannabinoid pathway can be engineered for use in a recombinant host cell of the present disclosure.

The heterologous nucleic acids encoding “helper” or “enhancer” proteins of the present disclosure can be incorporated (e.g., by recombinant transformation, or Cas9 integration) into a range of host cells already comprising a cannabinoid biosynthesis pathway to provide a system for enhanced production of cannabinoids (e.g., CBGA, CBDA, THCA, CBCA) or cannabinoid precursor compounds. Generally, the host cell used for the recombinant host cells of the present disclosure can be any cell that can be recombinantly modified with nucleic acids and then cultured to express the recombinant products of those nucleic acids, including polypeptides and metabolites produced by the activity of the recombinant polypeptides. A wide range of suitable sources of host cells are known in the art, and exemplary host cell sources useful as recombinant host cells of the present disclosure include, but are not limited to, Saccharomyces cerevisiae, Yarrowia lipolytica, Pichia pastoris, and Escherichia coli. It is also contemplated that the host cell source for a recombinant host cell of the present disclosure can include a non-naturally occurring cell source, e.g., an engineered host cell. For example, a non-naturally occurring source host cell, such as a yeast cell previously engineered for improved production of recombinant genes, may be used to prepare the recombinant host cell of the present disclosure. Accordingly, in at least one embodiment, the present disclosure provides a recombinant host cell previously engineered with an integrated functional cannabinoid biosynthesis pathway and a heterologous nucleic acid encoding an enhancer protein that is not part of the pathway, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli.

The recombinant host cells of the present disclosure comprise heterologous nucleic acids encoding a pathway of enzymes capable of producing a cannabinoid, such as CBGA, and a heterologous nucleic acid encoding an enhancer protein that is “off-pathway” (does not encode a pathway enzyme). As described elsewhere herein, cannabinoid pathway enzymes and the nucleic acid sequences encoding them are known in the art and provided herein, and can readily be used in accordance with the present disclosure. Typically, the nucleic acid sequence encoding enzymes in the cannabinoid pathway further include one or more nucleic acid sequences controlling expression of these cannabinoid pathway enzymes (e.g., promoter sequences). These one or more additional nucleic acid sequences together with the nucleic acid sequences encoding the enzymes which form a part of an cannabinoid biosynthetic pathway can be considered a heterologous nucleic acid sequence. A variety of techniques and methodologies are available and well known in the art for introducing such heterologous nucleic acid sequences encoding the cannabinoid pathway enzymes into a host cell so as to attain expression in the host cell. Techniques well known to the skilled artisan include, for example, those techniques found in the well-known Sambrook and Ausubel references cited elsewhere herein.

One of ordinary skill will recognize that the heterologous nucleic acids encoding the cannabinoid pathway enzymes and/or the nucleic acids encoding enhancer proteins as described herein can further comprise transcriptional promoters capable of controlling expression of the enzymes in the recombinant host cell. Generally, the transcriptional promoters are selected to be compatible with the host cell, so that promoters obtained from bacterial cells are used when a bacterial host cell is selected in accordance herewith, while a fungal promoter is used when a fungal host cell is selected, a plant promoter is used when a plant cell is selected, and so on.

Promoters useful in the recombinant host cells of the present disclosure may be constitutive or inducible, provided such promoters are operable in the host cells. Promoters that may be used to control expression in fungal host cells, such as Saccharomyces cerevisiae, are well known in the art and include, but are not limited to: inducible promoters, such as a Gal1 promoter or Gal10 promoter, a constitutive promoter, such as an alcohol dehydrogenase (ADH) promoter, a glyceraldehyde-3-phosphate dehydrogenase (GPD) promoter, or an S. pombe Nmt, or ADH promoter. Exemplary promoters that may be used to control expression in bacterial cells can include the Escherichia coli promoters lac, tac, trc, trp or the T7 promoter. Exemplary promoters that may be used to control expression in plant cells include, for example, a Cauliflower Mosaic Virus 35S promoter (Odell et al. (1985) Nature 313:810-812), a ubiquitin promoter (U.S. Pat. No. 5,510,474; Christensen et al. (1989)), or a rice actin promoter (McElroy et al. (1990) Plant Cell 2:163-171). Exemplary promoters that can be used in mammalian cells include, a viral promoter such as an SV40 promoter or a metallothionine promoter. All of these host cell promoters are well known by and readily available to one of ordinary skill in the art. Further nucleic acid control elements useful for controlling expression in a recombinant host cell can include transcriptional terminators, enhancers, and the like, all of which may be used with the heterologous nucleic acids incorporate in the recombinant host cells of the present disclosure.

A wide variety of techniques are well known in the art for linking transcriptional promoters and other control elements to heterologous nucleic acid sequences encoding pathway genes or other heterologous off-pathway nucleic acids. Such techniques are described in e.g., the Sambrook and Ausubel references cited elsewhere herein. Accordingly, in at least one embodiment, the heterologous nucleic acid sequences of the present disclosure comprise a promoter capable of controlling expression in a host cell, wherein the promoter is linked to a heterologous nucleic acid encoding an enhancer protein, and/or an enzyme of a cannabinoid pathway (e.g., AAE, OLS, OAC, PT4, or CBDAS). Such heterologous nucleic acid sequences can be integrated into a recombinant expression vector which ensures good expression in the desired host cell, wherein the expression vector is suitable for expression in a host cell, meaning that the recombinant expression vector comprises the heterologous nucleic acid sequence linked to any genetic elements required to achieve expression in the host cell. Genetic elements that may be included in the expression vector in this regard include a transcriptional termination region, one or more nucleic acid sequences encoding marker genes, one or more origins of replication, and the like. In some embodiments, the expression vector further comprises genetic elements required for the integration of the vector or a portion thereof in the host cell’s genome.

It is also contemplated that in some embodiments an expression vector comprising a heterologous nucleic acid encoding an enhancer protein of the present disclosure may further contain a marker gene. Marker genes useful in accordance with the present disclosure include any genes that allow the distinction of transformed cells from non-transformed cells, including all selectable and screenable marker genes. A marker gene may be a resistance marker such as an antibiotic resistance marker against, for example, kanamycin or ampicillin. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403).

Heterologous Nucleic Acids Encoding “Off Pathway” Enhancer Proteins that Improve Recombinant Cannabinoid Biosynthesis

As described elsewhere herein (including the Examples), screening of yeast transformed with heterologous nucleic acids that encode candidate enhancer proteins in a recombinant host system containing a biosynthetic pathway that converts hexanoic acid to the cannabinoid, CBGA, has identified numerous off-pathway proteins that substantially enhance production of CBGA. Table 2 below provides a summary description of exemplary off-pathway heterologous nucleic acids and encoded enhancer proteins that result in at least 1.5-fold increased CBGA yield in a recombinant host cell system, and their associated sequence identifiers. The nucleotide and amino acid sequences are included in the accompanying Sequence Listing.

TABLE 2

Enhancer Proteins for Recombinant Cannabinoid Biosynthesis

| Protein Type | GenBank Accession Annotation [Source Organism] | FIOPC¹ | SEQ ID NO: (nt) | SEQ ID NO: (aa) |
|---|---|---|---|---|
| Chaperonin | CCA36228.1 Chaperone of the endoplasmic reticulum lumen [Komagataella phaffii CBS 7435] | + | 15 | 16 |
| Chaperonin | NP_015084.1 Hsp90 family chaperone HSP82 [Saccharomyces cerevisiae S288C] | + | 17 | 18 |
| Chaperonin | NP_010138.1 Chaperonin-containing T-complex subunit CCT4 [Saccharomyces cerevisiae S288C] | +++ | 19 | 20 |
| Chaperonin | NP_012598.1 Chaperonin-containing T-complex subunit CCT5 [Saccharomyces cerevisiae S288C] | ++ | 21 | 22 |
| Efflux Pump | AGA23967.1 Small multidrug resistance efflux transporter EbrA [Bacillus subtilis subsp. subtilis str. BSP1] | +++ | 23 | 24 |
| Efflux Pump | WP_100109122.1 ATP-binding cassette domain-containing protein [Streptomyces peucetius] | + | 25 | 26 |
| Efflux Pump | AAC38085.1 VceA [Vibrio cholerae] | + | 27 | 28 |
| Efflux Pump | WP_132899240.1 Type I secretion system permease/ATPase [Pseudomonas aeruginosa] | + | 29 | 30 |
| Efflux Pump | WP_011015368.1 Multidrug efflux MFS transporter Cmr [Corynebacterium glutamicum] | +++ | 31 | 32 |
| Efflux Pump | WP_002246103.1 MULTISPECIES: multidrug efflux MATE transporter NorM [Neisseria] | + | 33 | 34 |
| Efflux Pump | AAD13557.1 LanJ [Streptomyces cyanogenus] | +++ | 35 | 36 |
| Efflux Pump | WP_047663068.1 Lipid A ABC transporter ATP-binding protein/permease MsbA [Escherichia coli] | + | 37 | 38 |
| Efflux Pump | WP_007055047.1, ABC transporter ATP-binding protein [Bifidobacterium longum] | + | 39 | 40 |
| LTPs | A0FGR8.1 Extended synaptotagmin-2 [Homo sapiens] | + | 41 | 42 |
| LTPs | NP_001183955.1 Pleckstrin homology domain-containing family A member 8 isoform 1 [Homo sapiens] | +++ | 43 | 44 |
| LTPs | NP_037369.1 | + | 45 | 46 |
| | PRELI domain-containing protein 1, mitochondrial isoform 1 precursor [Homo sapiens] | | | |
| LTPs | NP_892005.1 PRELI domain-containing protein 2 isoform a [Homo sapiens] | +++ | 47 | 48 |
| MEP | NP_180261.1 4-(cytidine 5′-phospho)-2-C-methyl-D-erithritol kinase [Arabidopsis thaliana] | ++ | 49 | 50 |
| MEP | NP_851233.1 4-hydroxy-3-methylbut-2-enyl diphosphate synthase [Arabidopsis thaliana] | + | 51 | 52 |
| MEP | XP_002511399.1 1-deoxy-D-xylulose 5-phosphate reductoisomerase, chloroplastic [Ricinus communis] | ++ | 53 | 54 |
| MEP | XP_002519102.1 4-hydroxy-3-methylbut-2-enyl diphosphate reductase, chloroplastic [Ricinus communis] | + | 55 | 56 |
| MEP | ABC74567.1 Acetoacetyl-CoA thiolase [Picrorhiza kurrooa] | + | 57 | 58 |
| MVA | NP_015297.1 Acetyl-CoA C-acetyltransferase [Saccharomyces cerevisiae S288C] | + | 59 | 60 |
| MVA | NP_014243.3 Nsg2p [Saccharomyces cerevisiae S288C] | + | 61 | 62 |
| Permease | QBG82552.1 Purine permease 1-like [Papaver somniferum] | + | 63 | 64 |
| Permease | XP_026416029.1 Purine permease 1-like [Papaver somniferum] | + | 65 | 66 |
| Protein Folding | CAC33587.1 Protein disulphide isomerase [Komagataella pastoris] | ++ | 67 | 68 |
| Protein Folding | CAY67364.1 Thiol oxidase required for oxidative protein folding in the endoplasmic reticulum [Komagataella phaffii GS115] | ++ | 69 | 70 |
| Protein Folding | CAY67758.1 bZIP transcription factor (ATF/CREB1 homolog) that regulates the unfolded protein response [Komagataella phaffii GS115] | + | 71 | 72 |
| Fatty acid pathway | CAA45738.1 Lipoxygenase [Oryza sativa Japonica Group] | + | 73 | 74 |
| Terpenoid Biosynthesis Pathway | ARE72261.1 Terpene synthase [Cannabis sativa] | + | 75 | 76 |
| Fatty acid pathway | CCA36228.1 Acetyl-CoA carboxylase (Acc1) [Saccharomyces cerevisiae S288C] | ++² | 77 | 78 |
| Fatty acid pathway | Synthetic Acc1 triple-mutant Acetyl-CoA carboxylase (Acc1) [Artificial] | +++² | 79 | 80 |

¹ “FIOPC” is “fold-improvement over to positive control” as determined by screening as described in Example 1. FIOPC ranges are indicated as follows: “+” >= 1.5-fold; “++” >= 2.0-fold; “+++” >= 2.5-fold ² FIOPC determined as described in Example 2.

All of the enhancer proteins encoded by heterologous nucleic acids shown in Table 2 were screened and observed to provide at least 1.5-fold improvement of CBGA production in a recombinant host cell system that converts hexanoic acid to CBGA via a pathway comprising the enzymes AAE, OLS, OAC and PT4 (see Table 1). These enhancer proteins are derived from a wide range of sources and carry out a range of functions that are not part of the four enzyme pathway for converting hexanoic acid to CBGA in the recombinant yeast cells. Accordingly, in at least one embodiment, the present disclosure provides a recombinant host cell comprising a pathway capable of producing a cannabinoid and a heterologous nucleic acid that encodes a protein not in the pathway, wherein the encoded protein is selected from: 1-deoxy-D-xylulose 5-phosphate reductoisomerase, chloroplastic [Ricinus communis]; 4-(cytidine 5′-phospho)-2-C-methyl-D-erithritol kinase [Arabidopsis thaliana]; 4-hydroxy-3-methylbut-2-enyl diphosphate reductase, chloroplastic [Ricinus communis]; 4-hydroxy-3-methylbut-2-enyl diphosphate synthase [Arabidopsis thaliana]; ABC transporter ATP-binding protein [Bifidobacterium longum]; Acetoacetyl-CoA thiolase [Picrorhiza kurrooa]; Acetyl-CoA C-acetyltransferase [Saccharomyces cerevisiae S288C]; ATP-binding cassette domain-containing protein [Streptomyces peucetius]; bZIP transcription factor (ATF/CREB1 homolog) that regulates the unfolded protein response [Komagataella phaffii GS115]; Chaperone of the endoplasmic reticulum lumen [Komagataella phaffii CBS 7435]; Chaperonin-containing T-complex subunit CCT4 [Saccharomyces cerevisiae S288C]; Chaperonin-containing T-complex subunit CCT5 [Saccharomyces cerevisiae S288C]; Extended synaptotagmin-2 [Homo sapiens]; Hsp90 family chaperone HSP82 [Saccharomyces cerevisiae S288C]; LanJ [Streptomyces cyanogenus]; Lipid A ABC transporter ATP-binding protein/permease MsbA [Escherichia coli]; Lipoxygenase [Oryza sativa Japonica Group]; Multidrug efflux MFS transporter Cmr [Corynebacterium glutamicum]; MULTISPECIES: multidrug efflux MATE transporter NorM [Neisseria]; Nsg2p [Saccharomyces cerevisiae S288C]; Pleckstrin homology domain-containing family A member 8 isoform 1 [Homo sapiens]; PRELI domain-containing protein 1, mitochondrial isoform 1 precursor [Homo sapiens]; PRELI domain-containing protein 2 isoform a [Homo sapiens]; Protein disulphide isomerase [Komagataella pastoris]; Purine permease 1-like [Papaver somniferum]; Purine permease 1-like [Papaver somniferum]; Small multidrug resistance efflux transporter EbrA [Bacillus subtilis subsp. subtilis str. BSP1]; Terpene synthase [Cannabis sativa]; Thiol oxidase required for oxidative protein folding in the endoplasmic reticulum [Komagataella phaffii GS115]; Type I secretion system permease/ATPase [Pseudomonas aeruginosa]; and VceA [Vibrio cholerae].

In at least one embodiment, the present disclosure provides a recombinant host cell comprising a pathway capable of producing a cannabinoid and a heterologous nucleic acid that encodes an enhancer protein that is not part of the pathway, wherein the nucleic acid comprises a nucleotide sequence having at least 90%, at least 95%, at least 96%, at least 97%, at least 98%, or at least 99% identity to any one of odd-numbered SEQ ID NOs: 15-79. In at least one embodiment, the nucleic acid comprises a nucleotide sequence of any one of odd-numbered SEQ ID NOs: 15-79.

In at least one embodiment, the present disclosure provides an isolated nucleic acid, wherein the nucleic acid encodes a polypeptide comprising an amino acid sequence having at least 90% identity to any one of even-numbered SEQ ID NOs: 16-80. In at least one embodiment, the nucleotide sequence of the isolated nucleic acid is codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli. In at least one embodiment, the isolated nucleic acid comprises a nucleotide sequence having at least 90% identity to any one of odd-numbered SEQ ID NOs: 15-79.

In at least one embodiment, the present disclosure provides a vector comprising a heterologous nucleic acid encoding a protein, wherein the nucleic acid encodes a polypeptide comprising an amino acid sequence having at least 90% identity to any one of even-numbered SEQ ID NOs: 16-80. In at least one embodiment, the vector comprises nucleic acid that is codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli. In at least one embodiment, the vector comprising the heterologous nucleic acid comprises a nucleotide sequence having at least 90% identity to any one of odd-numbered SEQ ID NOs: 15-79.

In at least one embodiment, the present disclosure provides a vector comprising a nucleic acid encoding a pathway of enzymes capable of producing a cannabinoid and a heterologous nucleic acid that encodes an enhancer protein not in the pathway, wherein the nucleic acid encodes a polypeptide comprising an amino acid sequence having at least 90% identity to any one of even-numbered SEQ ID NOs: 16-80. In at least one embodiment, the pathway capable of producing a cannabinoid comprises at least the exemplary enzymes AAE, OLS, OAC, and PT4, wherein the enzymes have the amino acid sequences of SEQ ID NO: 2 (AAE), SEQ ID NO: 4 (OLS), SEQ ID NO: 6 (OAC), and SEQ ID NO: 8 (PT4). In at least one embodiment, the vector comprises nucleic acid sequences encoding the pathway of enzymes that are codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli.

In at least one embodiment, the present disclosure provides a recombinant host cell comprising a pathway capable of producing a cannabinoid and a heterologous nucleic acid encoding an enhancer protein that is not in the pathway, wherein the nucleic acid encodes a polypeptide comprising an amino acid sequence having at least 80% identity to any one of even-numbered SEQ ID NOs: 16-80. In at least one embodiment, the nucleic acid encodes a polypeptide comprising an amino acid sequence having at least 85%, at least 90%, at least 95%, at least 96%, at least 97%, at least 98%, or at least 99% identity to any one of even-numbered SEQ ID NOs: 16-80.

The amino acid sequences of even-numbered SEQ ID NOs: 16-80 provided in the present disclosure begin with initiating methionine (M) residue at position 1, although it will be understood by the skilled artisan that this initiating methionine residue may be removed by biological processing machinery, such as in a host cell or in vitro translation system, to generate a mature protein lacking the initiating methionine residue. Accordingly, it is contemplated that any embodiment of the present disclosure comprising a protein of Table 2 can comprise an amino acid sequence of an even-numbered SEQ ID NOs: 16-80 wherein the methionine residue at position 1 is deleted.

As further described in the Examples, the off-pathway heterologous genes of Table 2 are capable of providing at least 1.5-fold, and up to 2-fold or greater increased cannabinoid production when introduced into a recombinant host cell comprising a heterologous cannabinoid biosynthesis pathway. Accordingly, in at least one embodiment, the present disclosure provides a recombinant host cell comprising a pathway capable of producing a cannabinoid and a heterologous nucleic acid encoding an enhancer protein that is not part of the pathway, wherein the recombinant host cell produces the cannabinoid with a titer that is increased at least 1.2-fold, at least 2-fold, at least 3-fold, at least 4-fold, at least 5-fold, or more relative to a control recombinant host cell comprising the pathway without the heterologous nucleic acid encoding the enhancer protein. For example, a control recombinant yeast cell that can convert hexanoic acid to CBGA but that has not been transformed with a heterologous nucleic acid encoding a protein of Table 2.

The nucleic acids identified in Table 2 encode a range of protein types that are not directly involved in the enzymatic pathway that converts hexanoic acid to CBGA in the recombinant host cell. The exemplary encoded proteins of Table 2 are of types including: a chaperonin, an efflux pump protein, a fatty acid pathway protein, a lipid transfer protein, a mevalonate pathway protein, a methylerythritol 4-phosphate pathway protein, a permease, and a terpenoid biosynthesis pathway protein. Accordingly, in at least one embodiment, the present disclosure provides a recombinant host cell comprising a pathway capable of producing a cannabinoid and a heterologous nucleic acid that encodes a protein not in the pathway, wherein the encoded protein is of a type selected from: a chaperonin, an efflux pump protein, a fatty acid pathway protein, a lipid transfer protein, a mevalonate pathway protein, a methylerythritol 4-phosphate pathway protein, a permease, and a terpenoid biosynthesis pathway protein.

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is a chaperonin; optionally, wherein the chaperonin is selected from: CCA36228.1 - Chaperone of the endoplasmic reticulum lumen [Komagataella phaffii CBS 7435] (SEQ ID NO: 16); NP_015084.1 - Hsp90 family chaperone HSP82 [Saccharomyces cerevisiae S288C] (SEQ ID NO: 18); NP_010138.1 - Chaperonin-containing T-complex subunit CCT4 [Saccharomyces cerevisiae S288C] (SEQ ID NO: 20); and NP_012598.1 - Chaperonin-containing T-complex subunit CCT5 [Saccharomyces cerevisiae S288C] (SEQ ID NO: 22).

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is an efflux pump protein; optionally, wherein the efflux pump protein is selected from: WP_100109122.1 - ATP-binding cassette domain-containing protein [Streptomyces peucetius] (SEQ ID NO: 26); AAC38085.1 - VceA [Vibrio cholerae] (SEQ ID NO: 28); WP_132899240.1 - Type I secretion system permease/ATPase [Pseudomonas aeruginosa] (SEQ ID NO: 30); WP_011015368.1 - Multidrug efflux MFS transporter Cmr [Corynebacterium glutamicum] (SEQ ID NO: 32); WP_002246103.1 - MULTISPECIES: multidrug efflux MATE transporter NorM [Neisseria] (SEQ ID NO: 34); AAD13557.1 - LanJ [Streptomyces cyanogenus] (SEQ ID NO: 36); WP_047663068.1 - Lipid A ABC transporter ATP-binding protein/permease MsbA [Escherichia coli] (SEQ ID NO: 38); and WP_007055047.1 - ABC transporter ATP-binding protein [Bifidobacterium longum] (SEQ ID NO: 40).

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is a lipid transfer protein (LTP); optionally, wherein the LTP is selected from: A0FGR8.1 - Extended synaptotagmin-2 [Homo sapiens] (SEQ ID NO: 42); NP_001183955.1 - Pleckstrin homology domain-containing family A member 8 isoform 1 [Homo sapiens] (SEQ ID NO: 44); NP_037369.1 - PRELI domain-containing protein 1, mitochondrial isoform 1 precursor [Homo sapiens] (SEQ ID NO: 46); and NP_892005.1 - PRELI domain-containing protein 2 isoform a [Homo sapiens] (SEQ ID NO: 48).

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is a methylerythritol 4-phosphate pathway (MEP) protein; optionally, wherein the MEP protein is selected from: NP_180261.1 - 4-(cytidine 5′-phospho)-2-C-methyl-D-erithritol kinase [Arabidopsis thaliana] (SEQ ID NO: 50); NP_851233.1 - 4-hydroxy-3-methylbut-2-enyl diphosphate synthase [Arabidopsis thaliana] (SEQ ID NO: 52); XP_002511399.1 - 1-deoxy-D-xylulose 5-phosphate reductoisomerase, chloroplastic [Ricinus communis] (SEQ ID NO: 54); XP_002519102.1 - 4-hydroxy-3-methylbut-2-enyl diphosphate reductase, chloroplastic [Ricinus communis] (SEQ ID NO: 56); and ABC74567.1 - Acetoacetyl-CoA thiolase [Picrorhiza kurrooa] (SEQ ID NO: 58).

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is a mevalonate pathway (MVA) protein; optionally, wherein the MVA protein is selected from: NP_015297.1 - Acetyl-CoA C-acetyltransferase [Saccharomyces cerevisiae S288C] (SEQ ID NO: 60); and NP_014243.3 - Nsg2p [Saccharomyces cerevisiae S288C] (SEQ ID NO: 62).

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is a permease; optionally, wherein the permease is selected from: QBG82552.1 - Purine permease 1-like [Papaver somniferum] (SEQ ID NO: 64); and XP_026416029.1 - Purine permease 1-like [Papaver somniferum] (SEQ ID NO: 66).

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is a protein folding protein; optionally, wherein the protein folding protein is selected from: CAC33587.1 - Protein disulphide isomerase [Komagataella pastoris] (SEQ ID NO: 68); CAY67364.1 - Thiol oxidase required for oxidative protein folding in the endoplasmic reticulum [Komagataella phaffii GS115] (SEQ ID NO: 70); and CAY67758.1 - bZIP transcription factor (ATF/CREB1 homolog) that regulates the unfolded protein response [Komagataella phaffii GS115] (SEQ ID NO: 72).

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is a fatty acid pathway protein; optionally, wherein the fatty acid pathway protein is selected from: CAA45738.1 - lipoxygenase [Oryza sativa Japonica Group] (SEQ ID NO: 74).

In at least one embodiment, the present disclosure provides a recombinant host cell comprising a pathway capable of producing a cannabinoid and a heterologous nucleic acid encoding a protein that is not part of the pathway, wherein the protein encoded by the heterologous nucleic acid is a terpenoid biosynthesis protein; optionally, wherein the terpenoid biosynthesis protein is selected from: ARE72261.1 - Terpene synthase [Cannabis sativa] (SEQ ID NO: 76).

Generally, the host cell source used in the recombinant host cell of the present disclosure can be any cell that can be recombinantly modified with nucleic acids and express the recombinant products of those nucleic acids, including polypeptides and metabolites produced by the activity of the recombinant polypeptides. A wide range of suitable sources of host cells are known in the art, and exemplary host cell sources useful as recombinant host cells of the present disclosure include, but are not limited to, Saccharomyces cerevisiae, Yarrowia lipolytica, Pichia pastoris, and Escherichia coli. It is also contemplated that the host cell source for a recombinant host cell of the present disclosure can include a non-naturally occurring cell source, e.g., an engineered host cell. For example, a non-naturally occurring source host cell, such as a yeast cell previously engineered for improved production of recombinant genes, may be used to prepare the recombinant host cell of the present disclosure. Accordingly, in at least one embodiment, the present disclosure provides a recombinant host cell transformed with a cannabinoid biosynthesis pathway and a heterologous nucleic acid encoding a protein that is not part of the pathway, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli.

### Use of Recombinant Host Cells in Preparation of Cannabinoids

As described elsewhere herein, the recombinant host cells provided by the present disclosure comprise a pathway capable of producing a cannabinoid and a heterologous nucleic acid encoding a protein not in the pathway. Furthermore, the recombinant host cells are capable of producing the cannabinoid with a titer that is increased (e.g., 1.5-fold or more) relative to a control recombinant host cell comprising the same pathway but without the heterologous nucleic acid encoding off-pathway protein. As described elsewhere herein, the heterologous nucleic acid can encode an exemplary enhancer protein as described in Table 2 and the associated Sequence Listing.

Although the cannabinoid pathways of FIGS. 1-2 depict the production of the more common naturally occurring cannabinoids, CBGA, THCA, CBDA, and CBCA, it is also contemplated that the recombinant host cells comprising cannabinoid pathways and a heterologous nucleic acid encoding an “off-pathway” enhancer protein, and associated methods of the present disclosure can also be used to biosynthesize a range of naturally occurring rare and/or synthetic cannabinoid compounds. Table 3 (below) depicts the names and structures of a wide range of exemplary cannabinoid compounds that are contemplated for production using the recombinant host cells and methods of the present disclosure.

TABLE 3

Cannabinoid compounds

| | | |
|---|---|---|
| Compound Name | Abbrev. Name | Chemical Structure |
| cannabigerolic acid | CBGA | 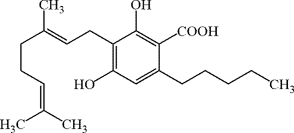 [SMILES: CCCCCc1cc(O)c(C/C=C(\C)CCC=C(C)C)c(O)c1C(=O)O] |
| cannabigerol | CBG | 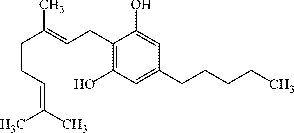 [SMILES: CCCCCc1cc(O)c(C/C=C(\C)CCC=C(C)C)c(O)c1] |
| Δ⁹-tetrahydrocannabinolic acid | Δ⁹-THCA | 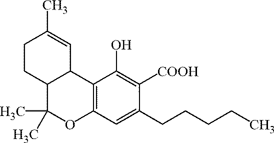 [SMILES: CCCCCc1cc2c(c(O)c1C(=O)O)C1C=C(C)CCC1C(C)(C)O2] |
| Δ⁹-tetrahydrocannabinol | Δ⁹-THC | 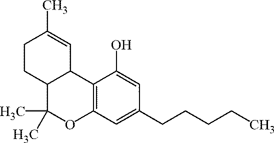 [SMILES: CCCCCc1cc(O)c2c(c1)OC(C)(C)C1CCC(C)=CC21] |
| Δ⁸-tetrahydrocannabinolic acid | Δ⁸-THCA | 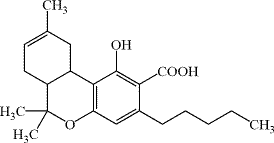 [SMILES: CCCCCc1cc2c(c(O)c1C(=O)O)C1CC(C)=CCC1C(C)(C)O2] |
| Δ⁸-tetrahydrocannabinol | Δ⁸-THC | 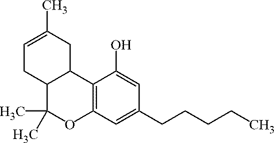 [SMILES: CCCCCc1cc(O)c2c(c1)OC(C)(C)C1CC=C(C)CC21] |
| cannabidiolic acid | CBDA | 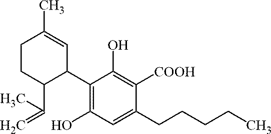 [SMILES: C=C(C)C1CCC(C)=CC1c1c(O)cc(CCCCC)c(C(=O)O)c1O] |
| cannabidiol | CBD | 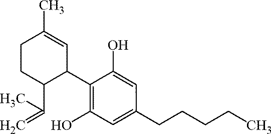 [SMILES: C=C(C)C1CCC(C)=CC1c1c(O)cc(CCCCC)cc1O] |
| cannabichromenic acid | CBCA | 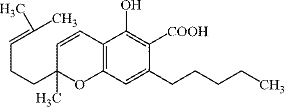 [SMILES: CCCCCc1cc2c(c(O)c1C(=O)O)C=CC(C)(CCC=C(C)C)O2] |
| cannabichromene | CBC | 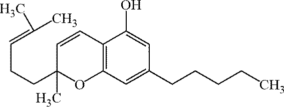 [SMILES: CCCCCc1cc(O)c2c(c1)OC(C)(CCC=C(C)C)C=C2] |
| cannabinolic acid | CBNA | 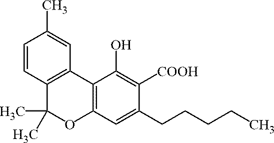 [SMILES: CCCCCc1cc2c(c(O)c1C(=O)O)-c1cc(C)ccc1C(C)(C)O2] |
| cannabinol | CBN | 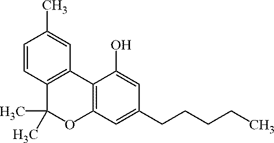 [SMILES: CCCCCc1cc(O)c2c(c1)OC(C)(C)c1ccc(C)cc1-2] |
| cannabidivarinic acid | CBDVA | 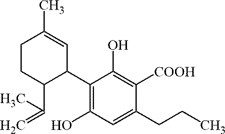 [SMILES: C=C(C)C1CCC(C)=CC1c1c(O)cc(CCC)c(C(=O)O)c1O] |
| cannabidivarin | CBDV | 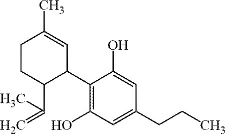 [SMILES: C=C(C)C1CCC(C)=CC1c1c(O)cc(CCC)cc1O] |
| Δ⁹- tetrahydrocannabivarinic acid | Δ⁹-THCVA | 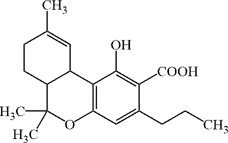 [SMILES: CCCc1cc2c(c(O)c1C(=O)O)C1C=C(C)CCC1C(C)(C)O2] |
| Δ⁹- tetrahydrocannabivarin | Δ⁹-THCV | 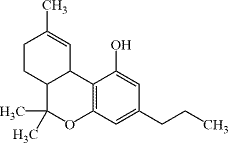 [SMILES: CCCc1cc(O)c2c(c1)OC(C)(C)C1CCC(C)=CC21] |
| Cannabidibutolic acid | CBDBA | 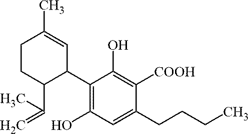 [SMILES: C=C(C)C1CCC(C)=CC1c1c(O)cc(CCCC)c(C(=O)O)c1O] |
| Cannabidibutol | CBDB | 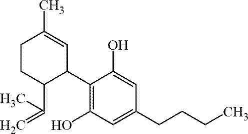 [SMILES: C=C(C)C1CCC(C)=CC1c1c(O)cc(CCCC)cc1O] |
| Δ⁹- tetrahydrocannabutolic acid | Δ⁹-THCBA | 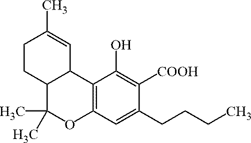 [SMILES: CCCCc1cc2c(c(O)c1C(=O)O)C1C=C(C)CCC1C(C)(C)O2] |
| Δ⁹- tetrahydrocannabutol | Δ⁹-THCB | 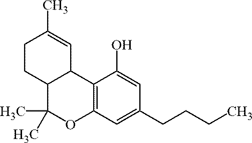 [SMILES: CCCCc1cc(O)c2c(c1)OC(C)(C)C1CCC(C)=CC21] |
| Cannabidiphorolic acid | CBDPA | 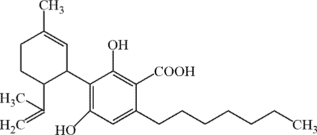 [SMILES: C=C(C)C1CCC(C)=CC1c1c(O)cc(CCCCCCC)c(C(=O)O)c1O] |
| Cannabidiphorol | CBDP | 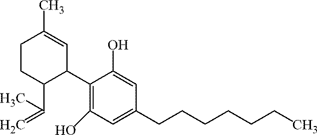 [SMILES: C=C(C)C1CCC(C)=CC1c1c(O)cc(CCCCCCC)cc1O] |
| Δ⁹- tetrahydrocannabiphorolic acid | Δ⁹-THCPA | 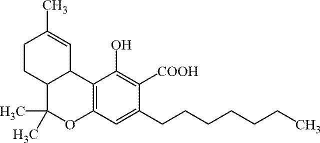 [SMILES: CCCCCCCc1cc2c(c(O)c1C(=O)O)C1C=C(C)CCC1C(C)(C)O2] |
| Δ⁹- tetrahydrocannabiphorol | Δ⁹-THCP | 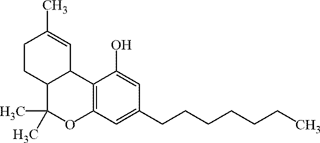 [SMILES: CCCCCCCc1cc(O)c2c(c1)OC(C)(C)C1CCC(C)=CC21] |
| cannabichromevarinic acid | CBCVA | 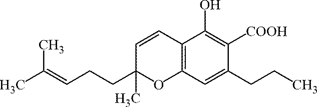 [SMILES: CCCc1cc2c(c(O)c1C(=O)O)C=CC(C)(CCC=C(C)C)O2] |
| cannabichromevarin | CBCV | 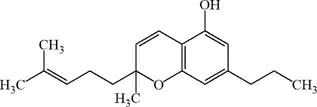 [SMILES: CCCc1cc(O)c2c(c1)OC(C)(CCC=C(C)C)C=C2] |
| cannabigerovarinic acid | CBGVA | 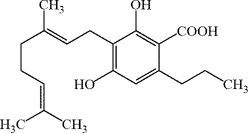 [SMILES: CCCc1cc(O)c(C/C=C(\C)CCC=C(C)C)c(O)c1C(=O)O] |
| cannabigerovarin | CBGV | 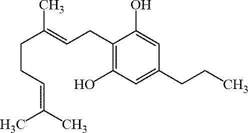 [SMILES: CCCc1cc(O)c(C/C=C(\C)CCC=C(C)C)c(O)c1] |
| cannabicyclolic acid | CBLA | 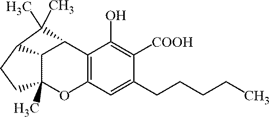 |
| cannabicyclol | CBL | 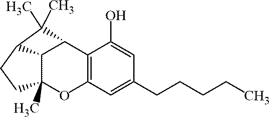 |
| cannabielsoinic acid | CBEA | 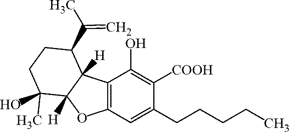 [SMILES: C=C(C)[C@@H]1CC[C@](C)(O)[C@H]2Oc3cc(CCCCC)c(C(=O)O)c(O)c3[C@@H]12] |
| cannabielsoin | CBE | 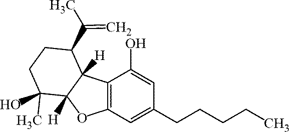 [SMILES: C=C(C)[C@@H]1CC[C@](C)(O)[C@H]2Oc3cc(CCCCC)cc(O)c3[C@@H]12] |
| cannabicitranic acid | CBTA | 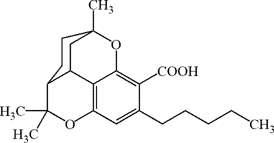 [SMILES: CCCCCc1cc2c3c(c1C(=O)O)OC1(C)CCC(C3C1)C(C)(C)O2] |
| cannabicitran | CBT | 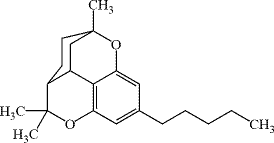 [SMILES: CCCCCc1cc2c3c(c1)OC(C)(C)C1CCC(C)(CC31)O2] |

In at least one embodiment, it is also contemplated that the heterologous nucleic acids encoding an enhancer protein of the present disclosure (e.g., as in Table 2) can be introduced into a recombinant host cell to provide a method for the improved biosynthesis of cannabinoid precursor compounds or cannabinoid precursor derivatives in terms of titer, yield, and production rate. Cannabinoid precursors, or cannabinoid precursor derivatives can include, but are not limited to olivetolic acid, olivetol, divarin, PDAL, HTAL, GPP, polyketides, polyketide derivatives, and others known in the art (see e.g., Elsohly and Slade, Life Sci. 2005 Dec. 22; 78(5):539-48. Epub 2005 Sep. 30; Bow, E. W. and Rimoldi, J. M., “The Structure-Function Relationships of Classical Cannabinoids: CB1/CB2 Modulation,” Perspectives in Medicinal Chemistry 2016:8 17-39 doi: 10.4137/PMC.S32171.) Such precursor compounds can be useful products, and/or can be used to prepare other derivative compounds, either synthetically or biosynthetically. In at least one embodiment, a cannabinoid precursor compound, such as OA or divarinic acid (DA), can be produced, and then further modified or derivatized using an in vitro enzymatic biosynthesis, using e.g., a cannabinoid synthase.

In at least one embodiment, the present disclosure provides a method for producing a cannabinoid or cannabinoid precursor comprising: (a) culturing in a suitable medium a recombinant host cell of the present disclosure; and (b) recovering the produced cannabinoid or cannabinoid precursor.

In at least one embodiment of the method for producing a cannabinoid, a heterologous nucleic acid encoding an enhancer protein of Table 2, can be introduced into a recombinant host cell comprising a pathway capable of producing a cannabinoid (e.g., CBGA) to provide an recombinant host cell that has improved biosynthesis of the cannabinoid in terms of titer, yield, and production rate.

In at least one embodiment, a recombinant host cell of the present disclosure can produce a cannabinoid compound, or a composition comprising a cannabinoid compound, wherein the cannabinoid is selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof. In at least one embodiment, a recombinant host cell of the present disclosure can be used to produce a cannabinoid selected from cannabigerolic acid (CBGA), cannabidiolic acid (CBDA), cannabichromenic acid (CBCA), and any combination thereof.

In at least one embodiment of the method for producing a cannabinoid, the method can further comprise contacting a cell-free extract of the culture containing the produced cannabinoid with a biocatalytic reagent or chemical reagent.

In at least one embodiment, the biocatalytic reagent is an enzyme capable of converting the produced cannabinoid to a different cannabinoid or a cannabinoid derivative compound. In at least one embodiment, the chemical reagent is capable of chemically modifying the produced cannabinoid to produce a different cannabinoid or a cannabinoid derivative compound.

Accordingly, in at least one embodiment of the method, the recombinant host cell with improved cannabinoid production in terms of titer, yield, and production rate can be used in the production of a cannabinoid (see e.g., compounds of Table 3), or a cannabinoid derivative compound. Cannabinoid derivative compounds can include a wide range of naturally-occurring and non-naturally occurring compounds.

Cannabinoid derivative compounds produced using the recombinant host cells of the present disclosure can include any compound structurally related to a cannabinoid compound (e.g., compounds of Table 3) but which lacks one or more of the chemical moieties present in the cannabinoid compound from which it derives. Exemplary chemical moieties that may be lacking in a cannabinoid derivative include, but are not limited to, methyl, alkyl, alkenyl, methoxy, alkoxy, acetyl, carboxyl, carbonyl, oxo, ester, hydroxyl, aryl, heteroaryl, cycloalkyl, cycloalkenyl, cycloalkylalkenyl, cycloalkenylalkyl, cycloalkenylalkenyl, heterocyclylalkenyl, heteroarylalkenyl, arylalkenyl, heterocyclyl, aralkyl, cycloalkylalkyl, heterocyclylalkyl, heteroarylalkyl, and the like.

Alternatively, cannabinoid derivative compounds using the recombinant host cells of the present disclosure can include one or more additional chemical moieties not present in the cannabinoid compound from which it derives. Exemplary chemical moieties that may be added in a cannabinoid derivative include, but are not limited to azido, halo (e.g., chloride, bromide, iodide, fluorine), methyl, alkyl, alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, spirocyclyl, heterospirocyclyl, heterocyclyl, thioalkyl, sulfone, sulfonyl, sulfoxide, amino, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

Accordingly, in at least one embodiment, the present disclosure provides a method of producing a cannabinoid derivative, wherein the method comprises: (a) culturing in a suitable medium a recombinant host cell of the present disclosure; and (b) recovering the produced cannabinoid derivative. In at least one embodiment, the method of producing a cannabinoid derivative further contacting a cell-free extract of the culture containing the produced cannabinoid with a biocatalytic reagent or chemical reagent capable of converting the cannabinoid to a cannabinoid derivative. In at least one embodiment, the biocatalytic reagent is an enzyme capable of converting the produced cannabinoid to a different cannabinoid or a cannabinoid derivative compound. In at least one embodiment, the chemical reagent is capable of chemically modifying the produced cannabinoid to produce a different cannabinoid or a cannabinoid derivative compound.

Cannabinoid derivatives that can be produced with improved yield using a recombinant host cell of the present disclosure can include cannabinoid derivatives modified (e.g., biocatalytically or synthetically) to provide improved properties of pharmaceutical metabolism and/or pharmacokinetics (e.g. solubility, bioavailability, absorption, distribution, plasma half-life and metabolic clearance). Modifications typically providing such improved pharmaceutical properties can include, but are not limited to, halogenation, acetylation and methylation.

It is also contemplated that the cannabinoids and cannabinoid derivatives produced by the methods disclosed herein can include pharmaceutically acceptable isotopically labeled cannabinoid and cannabinoid derivative compounds. For example, cannabinoid and cannabinoid derivative compounds wherein the hydrogen atoms are replaced or substituted by one or more deuterium or tritium atoms. Such isotopically labeled cannabinoids and derivatives can be useful in studies of in vivo pharmacokinetics and tissue distribution.

Upon production by the host cells or in the cell-free mixture of the cannabinoid precursors or cannabinoids in accordance with the recombinant host cells, and methods of the present disclosure, the desired compounds may be recovered from the host cell suspension or cell-free mixture and separated from other constituents, such as media constituents, cellular debris, etc. Techniques for separation and recovery of the desired compounds are known to those of skill in the art and can include, for example, solvent extraction (e.g. butane, chloroform, ethanol), column chromatography-based techniques, high-performance liquid chromatography (HPLC), for example, and/or countercurrent separation (CCS) based systems. The recovered cannabinoid compounds may be obtained in a more or less pure form, for example, the desired cannabinoid compound of purity of at least about 60% (w/v), about 70% (w/v), about 80% (w/v), about 90% (w/v), about 95% (w/v) or about 99% (w/v).

It is contemplated that the cannabinoid, cannabinoid precursor, cannabinoid precursor derivative, or cannabinoid derivative recovered using the methods of the present disclosure can be in the form of a salt. In at least one embodiment, the recovered salt of the cannabinoid, cannabinoid precursor, cannabinoid precursor derivative, or cannabinoid derivative is a pharmaceutically acceptable salt. Such pharmaceutically acceptable salts retain the biological effectiveness and properties of the free base compound.

As described elsewhere herein, the rare or synthetic derivatives of cannabinoid compounds that can be produced by the recombinant host cells and methods of the present disclosure are contemplated to exhibit biological and pharmacological properties like those of the more well-studied cannabinoids such as THC and CBD. Accordingly, in at least one embodiment, the present disclosure also provides a composition comprising a rare or synthetic cannabinoid, such as a varin cannabinoid, prepared using the recombinant host cells and methods disclosed herein. It is contemplated that the rare cannabinoid compositions provided by the recombinant host cells and methods of the present disclosure can include pharmaceutical compositions, food compositions, and beverage compositions, containing a rare cannabinoid. Generally, compositions comprising rare cannabinoid compounds can further comprise any of the well-known vehicles, excipients, and auxiliary substances, used in the art of formulating pharmaceutical, food, or beverage compositions, such as wetting or emulsifying agents, pH buffering substances and the like. For example, pharmaceutical compositions can contain any of the typical pharmaceutically acceptable excipients including, but not limited to, liquids such as water, saline, polyethylene glycol, hyaluronic acid, glycerol, and ethanol. Pharmaceutically acceptable salts can also be included therein, for example, mineral acid salts such as hydrochlorides, phosphates, sulfates, and the like; and the salts of organic acids such as acetates, propionates, benzoates, and the like. In at least one embodiment, a pharmaceutical composition can comprise a pharmaceutically acceptable excipient that serves as a stabilizer of the rare cannabinoid composition. Examples of suitable excipients that also act as stabilizers include, without limitation, pharmaceutical grades of dextrose, sucrose, lactose, sorbitol, inositol, dextran, and the like. Other suitable pharmaceutical excipients can include, without limitation, starch, cellulose, sodium or calcium phosphates, citric acid, glycine, polyethylene glycols (PEGs), and combinations thereof.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Generation and Screening of Proteins That Enhance CBGA Biosynthesis in Saccharomyces Cerevisiae

This example illustrates the generation and screening of a library heterologous nucleic encoding proteins that can enhance biosynthesis of the cannabinoid, CBGA. As described herein, the heterologous nucleic acids were transformed into a strain of Saccharomyces cerevisiae capable of biosynthesizing CBGA via a pathway comprising genes encoding the enzymes AAE (SEQ ID NO: 2), OLS (SEQ ID NO: 4), OAC (SEQ ID NO: 6), and PT4 (SEQ ID NO: 10). The transformants were screened for enhanced CBGA production. The library of “enhancer” or “helper” proteins identified as providing enhanced CBGA biosynthesis provides a useful tool for enhancing cannabinoid biosynthesis in yeast and other recombinant host cells.

### Materials and Methods

### A. Enhancer Protein Library Creation

Candidate proteins for improving biosynthesis of CBGA from hexanoic acid in the recombinant yeast host already transformed with the pathway comprising the enzymes AAE (SEQ ID NO: 2), OLS (SEQ ID NO: 4), OAC (SEQ ID NO: 6), and PT4 (SEQ ID NO: 10) were selected based on a variety of potential mechanistic rationales. A summary of the Categories of Candidate Enhancer Proteins and their Predicted Mechanisms for Improving CBGA Production are provided in Table 4.

TABLE 4

| Protein Type | Mechanistic Rationale for Improved CBGA Production | No. of Candidate Proteins Selected |
|---|---|---|
| Chaperonin | Provide favorable conditions for correct folding and prevent aggregation of proteins involved in cannabinoid synthesis in microbial hosts. | 10 |
| Efflux Pump | Assist microbial host in pumping out biosynthesized cannabinoids and thereby reduce their toxicity to the host. | 40 |
| Fatty Acid Pathway | Help improve biosynthesis of cannabinoid precursors (e.g., hexanoic acid) in microbial hosts. | 3 |
| Lipid Transfer Proteins (LTPs) | Assist transfer of cannabinoids from cytoplasm to cell membrane in microbial hosts. | 12 |
| Methylerythritol 4-Phosphate Pathway (MEP) | Provide an improved metabolic pathway for GPP biosynthesis that bypasses microbial host’s regulation of mevalonate pathway. | 6 |
| Mevalonate Pathway (MVA) | Provide an improved MVA pathway in the microbial that enhances GPP biosynthesis. | 9 |
| Permease | Permeases are known to transport adenine and cytokinin hormones, and non-purine alkaloids, and could potentially act as cannabinoid transporters in microbial hosts. | 16 |
| Protein Folding | Provide increased levels of correctly folded recombinant proteins in microbial hosts. | 11 |
| Terpenoid Biosynthesis Pathway | Terpenoid synthases are known to catalyze a wide range of reactions involved in terpenoid biosynthesis, and could potentially act as catalysts in the biosynthesis of cannabinoids in microbial hosts. | 6 |

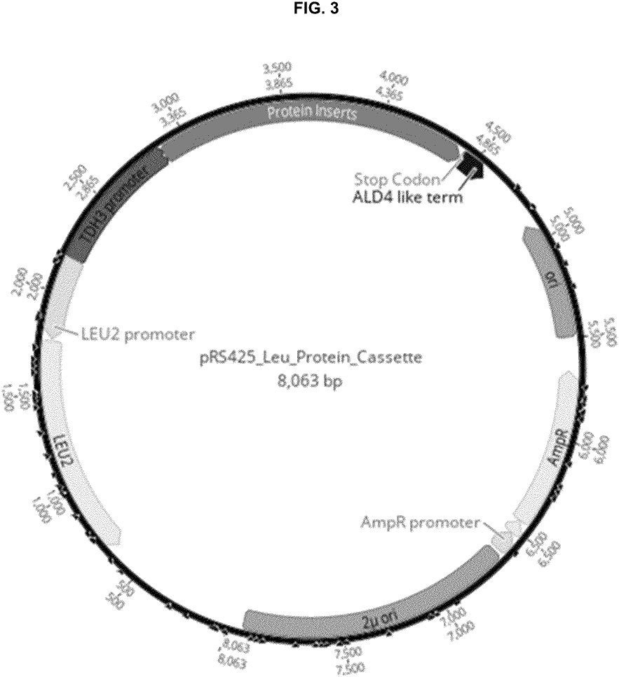

From the 9 categories of proteins listed in Table 4, a total of 113 candidate enhancer proteins were identified from various organisms. The nucleic acid sequence encoding each these candidate enhancer proteins was codon-optimized and synthesized with a 5′ flanking region to TDH3 promoter and a 3′ flanking region to ALD4 terminator. Each of the 113 codon-optimized sequences were cloned as a “Protein Insert” into the yeast expression vector with auxotrophic marker (LEU2 gene) shown in FIG. 3. The vector also included the constitutive TDH3 promoter to drive expression of the candidate enhancer protein.

### B. Expression of Enhancer Proteins in Yeast

Each enhancer protein was individually transformed into a Saccharomyces cerevisiae strain that had a cannabinoid pathway for biosynthesis of CBGA from hexanoic acid which included genes encoding the following enzymes derived from Cannabis sativa expressed under constitutive promoters: AAE (SEQ ID NO: 2), OLS (SEQ ID NO: 4), OAC (SEQ ID NO: 6), and PT4 (SEQ ID NO: 10). The TDH3 promoter of the transformed yeast expression vector was used to express the enhancer protein. Transformed yeast strains were plated on selective SC-LEU plates and 1-4 colonies were picked for each of the 113 enhancer protein transformants.

### C. Screening for Enhanced CBGA Biosynthesis

Transformed yeast strains were grown in 0.3 mL SC-LEU selective media for 48 h at 30 C. The yeast strain with the CBGA pathway but an empty vector (without enhancer protein) was used as the control strain. The cultures were then inoculated to 0.3 mL SC-LEU selective media with 0.2 mM hexanoic acid to grow for 48 h at 30 C. The whole broth of the culture was then extracted with 0.26 mL acetonitrile and measured by mass spectrometry. The concentration of CBGA was quantified by referring to a standard curve. The variants with over 1.5-fold improvement of CBGA production compared to the control strain were selected as hits.

### Results

Of the 113 selected candidate enhancer proteins, 27% (30 variants) showed at least 1.5-fold improvement and 11% (12 variants) showed at least 2-fold improvement in terms of CBGA production relative to the control strain expressing an empty vector. The distribution of candidate enhancer proteins identified across the 9 different protein types considered is summarized in Table 4. The specific source, protein type, and sequence of the 30 enhancer proteins identified as capable of at least 1.5-fold improved CBGA titer production are summarized in Table 2.

### Example 2: Acc1 Protein That Enhances CBGA Biosynthesis in Saccharomyces Cerevisiae

This example illustrates the generation of a yeast strain with a heterologous nucleic encoding the fatty acid pathway protein, acetyl-CoA carboxylase (Acc1) of SEQ ID NO: 78, and a Acc1 triple-mutant, Acc1_3p of SEQ ID NO: 80, and screening the strains for enhanced biosynthesis of the cannabinoid, CBGA. The heterologous nucleic acids encoding wild-type Acc1 and the triple mutant (S659A/S686A/S1157A) variant of Acc1, Acc1_3p were transformed into a strain of Saccharomyces cerevisiae capable of biosynthesizing CBGA via a pathway comprising genes encoding the enzymes AAE (SEQ ID NO: 2), OLS (SEQ ID NO: 4), OAC (SEQ ID NO: 6), and PT4 (SEQ ID NO: 10). The transformants were screened for enhanced CBGA production.

### Materials and Methods

### A. Acc1 and Acc1 3p Integration Constructs

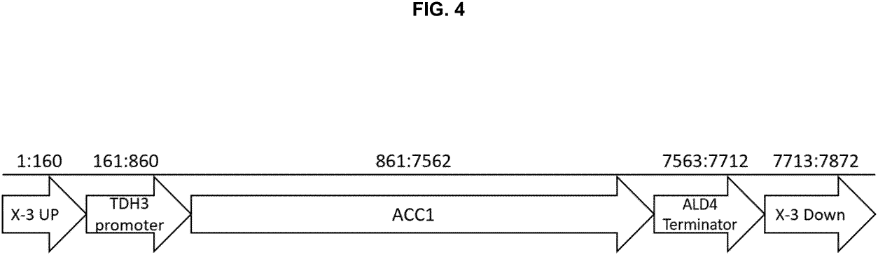

Wild-type Acc1 protein was amplified from Saccharomyces cerevisiae genomic DNA with 5′ flanking region to TDH3 promoter (FP-TDH3p-ACC1; SEQ ID NO: 81) and a 3′ flanking region to ALD4 terminator (RP-ACC1-ALD4t; SEQ ID NO: 88). Overlapping primers encoding serine to alanine mutations were used to amplified Acc1 to create fragments containing mutations (FP-TDH3p-ACC1, SEQ ID NO:81; RP-ACC1-S659A, SEQ ID NO: 82; FP-ACC1-S659A, SEQ ID NO: 83; RP-ACC1-S686A, SEQ ID NO: 84; FP-ACC1-S686A, SEQ ID NO: 85; RP-ACC1-S1157A, SEQ ID NO: 86; FP-ACC1-S1157A, SEQ ID NO: 87; RP-ACC1-ALD4t, SEQ ID NO: 88). The fragments were assembled together by OE-PCR (FP-TDH3p-ACC1, SEQ ID NO: 81; RP-ACC1-ALD4t, SEQ ID NO: 88) to generate ACC1_3p gene variant. Saccharomyces cerevisiae TDH3 promoter and ALD4 terminator were amplified from genomic DNA using Ultramer® primers containing the X-3 locus of chromosome 10 (6000000888_RP_TDH3p, SEQ ID NO: 89; 6000001376_FP_Ultramer_X3UP_HELPER, SEQ ID NO: 90; 6000000889_FP_ALD4t, SEQ ID NO: 91; 6000001377_RP_Ultramer_HELPER_X3DN, SEQ ID NO: 92). The promoter and terminator containing the X-3 locus were added onto the Acc1 and Acc1_3p by OE-PCR (6000001374_FP_Rescue_X3UP, SEQ ID NO: 93; 6000001375_RP_Rescue_X3DN, SEQ ID NO: 94). A schematic representation of the resulting expression cassette used to target the X-3 locus of chromosome 10 (Chr X: 223616..224744) is shown in FIG. 4.

### B. Acc1 and Acc1 3p Integration in Yeast

Each integration constructs were transformed into a Saccharomyces cerevisiae strain that already had integrated a cannabinoid biosynthesis pathway, which included the following genes from Cannabis sativa expressed under constitutive promoters: AAE1, PKS, OAC, and PT.

### C. Screening Transformants for Cannabinoid Biosynthesis

The resulting transformed yeast strains were grown in 0.3 mL YP + 2% glucose media for 48 h at 30° C. An untransformed yeast strain was used as the control strain. The cultures were then inoculated with YP + 2% glucose and 0.2 mM hexanoic acid to grow for 48 h at 30° C. The whole broth of the culture was then extracted with 0.26 mL acetonitrile and measured by mass spectrometry. The OA and CBGA concentrations were quantified based on a standard curve.

### Results

As shown by the results summarized in Table 5, exemplary yeast strains integrated with the heterologous nucleic acid encoding the wild-type Acc1 of SEQ ID NO: 78 showed up to a 5-fold improvement in OA production and up to a 2-fold improvement in CBGA production compared to that of the untransformed control yeast strain. However, yeast strains integrated with the heterologous nucleic acid encoding the triple-mutant variant, Acc1_3p of SEQ ID NO: 80 showed up to 24-fold improvement in OA production and up to a 2.8-fold improvement in CBGA production compared to that of the untransformed control strain.

TABLE 5

| Strain No. | OA Production FIOPC | CBGA Production FIOPC |
|---|---|---|
| Acc1 3p | | |
| Build248_31 | 6.976 | 2.838 |
| Build248_33 | 7.408 | 2.459 |
| Build248_9 | 23.899 | 1.696 |
| Build248_10 | 18.187 | 1.694 |
| Build248_11 | 17.407 | 1.647 |
| Build248_7 | 17.395 | 1.598 |
| Build248_29 | 14.809 | 1.552 |
| Build248_27 | 13.579 | 1.499 |
| Build248_18 | 10.545 | 1.363 |
| Build248_3 | 16.509 | 1.361 |
| Build248_1 | 6.172 | 1.198 |
| Build248_15 | 1.949 | 1.166 |
| Build248_32 | 1.927 | 1.110 |
| Build248_2 | 0.855 | 1.059 |
| Build246_10 | 5.411 | 2.085 |
| Build246_18 | 4.045 | 2.043 |
| Build246_15 | 2.425 | 1.198 |
| Build246_34 | 1.584 | 1.109 |
| Build246_7 | 1.228 | 1.108 |
| Build246_19 | 1.721 | 0.927 |
| Build246_40 | 0.334 | 0.826 |
| Build246_11 | 0.489 | 0.822 |
| Build246_6 | 0.312 | 0.814 |
| Build246_27 | 0.396 | 0.813 |
| Build246_35 | 0.793 | 0.805 |
| Build246_23 | 0.555 | 0.800 |
| Build246_16 | 0.634 | 0.799 |
| Build246_5 | 0.273 | 0.789 |

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A recombinant host cell comprising a pathway of enzymes capable of converting hexanoic acid to CBGA and a heterologous nucleic acid that encodes a protein not in the pathway, wherein
the encoded protein is derived from a source that is not Cannabis sativa or Saccharomyces cerevisiae and is of a type selected from: a chaperonin, an efflux pump protein, a fatty acid pathway protein, a lipid transfer protein, a mevalonate pathway protein, a methylerythritol 4-phosphate pathway protein, a permease, and a terpenoid biosynthesis pathway protein; and
production of the cannabinoid is increased at least 1.5-fold relative to a control recombinant host cell comprising the pathway and without the heterologous nucleic acid.

2. (canceled)

3. (canceled)

4. The cell of claim 1, wherein the encoded protein is derived from a source selected from: Arabidopsis thaliana, Bacillus subtilis, Bifidobacterium longum, Cannabis sativa, Corynebacterium glutamicum, Escherichia coli, Homo sapiens, Komagataella pastoris, Komagataella phaffii, Neisseria, Oryza sativa, Papaver somniferum, Picrorhiza kurrooa, Pseudomonas aeruginosa, Ricinus communis, Saccharomyces cerevisiae, Streptomyces cyanogenus, Streptomyces peucetius, and Vibrio cholerae.

5. The cell of claim 1, wherein the encoded protein comprises an amino acid sequence having at least 90% identity to any one of even numbered SEQ ID NOs: 16-80.

6. The cell of claim 1, wherein the heterologous nucleic acid comprises a nucleotide sequence having at least 90% identity to any one of odd-numbered SEQ ID NOs: 15-79.

7. The cell of claim 1, wherein recombinant host cell source is selected from Saccharomyces cerevisiae, Yarrowia lipolytica, Pichia pastoris, and Escherichia coli.

8. The cell of claim 1, wherein the pathway comprises enzymes capable of catalyzing reactions (i) - (iv): 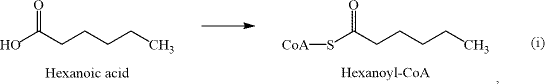 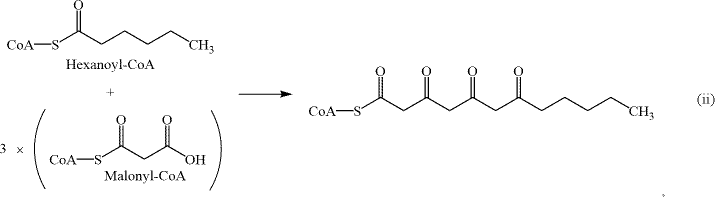 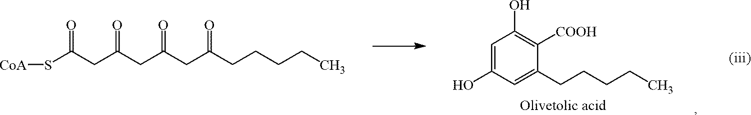
and 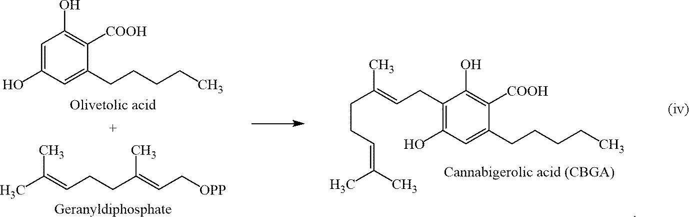
.

9. The cell of claim 1, wherein the pathway comprises at least the enzymes AAE, OLS, OAC, and PT4; optionally, wherein the enzymes AAE, OLS, OAC, and PT4 have an amino acid sequence of at least 90% identity to SEQ ID NO: 2 (AAE), SEQ ID NO: 4 (OLS), SEQ ID NO: 6 (OAC), and SEQ ID NO: 8 or 10 (PT4), respectively.

10. The cell of claim 1, wherein the pathway further comprises an enzyme capable of catalyzing the conversion of CBGA to Δ⁹-THCA, CBDA, and/or CBCA.

11. The cell of claim 8, wherein the pathway comprises: (i) a CBDA synthase having an amino acid sequence of at least 90% identity to SEQ ID NO: 12 or 14; or (ii) a THCA synthase having an amino acid sequence of at least 90% identity to SEQ ID NO: 96 or 98.

12. A method for producing a cannabinoid comprising:
(a) culturing in a suitable medium a recombinant host cell comprising a pathway of enzymes capable of converting hexanoic acid to CBGA and a heterologous nucleic acid that encodes a protein not in the pathway, wherein the encoded protein is derived from a source that is not Cannabis sativa or Saccharomyces cerevisiae and is of a type selected from: a chaperonin, an efflux pump protein, a fatty acid pathway protein, a lipid transfer protein, a mevalonate pathway protein, a methylerythritol 4-phosphate pathway protein, a permease, and a terpenoid biosynthesis pathway protein; and
(b) recovering the produced cannabinoid.

13. The method of claim 10, wherein production of the cannabinoid is increased at least 1.5-fold relative to a control recombinant host cell comprising the pathway and without the heterologous nucleic acid.

14. The method of claim 10, wherein the encoded protein is derived from a source selected from: Arabidopsis thaliana, Bacillus subtilis, Bifidobacterium longum, Cannabis sativa, Corynebacterium glutamicum, Escherichia coli, Homo sapiens, Komagataella pastoris, Komagataella phaffii, Neisseria, Oryza sativa, Papaver somniferum, Picrorhiza kurrooa, Pseudomonas aeruginosa, Ricinus communis, Saccharomyces cerevisiae, Streptomyces cyanogenus, Streptomyces peucetius, and Vibrio cholerae.

15. The method of claim 10, wherein the encoded protein comprises an amino acid sequence having at least 90% identity to any one of even numbered SEQ ID NOs: 16-80.

16. The method of claim 10, wherein the heterologous nucleic acid comprises a nucleotide sequence having at least 90% identity to any one of odd-numbered SEQ ID NOs: 15-79.

17. The method of claim 10, wherein the recombinant host cell is a cell derived from a source selected from: Saccharomyces cerevisiae, Escherichia coli, Yarrowia lipolytica, and Pichia pastoris.

18. The method of claim 10, wherein the pathway comprises enzymes capable of catalyzing reactions (i) - (iv): 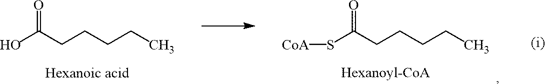 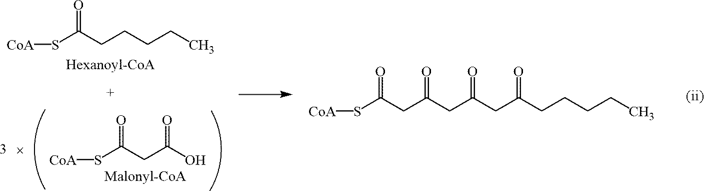 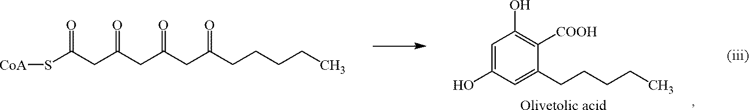
and 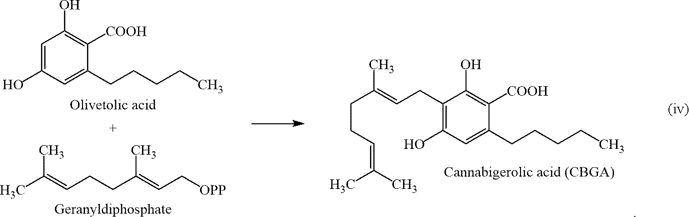
.

19. The method of claim 10, wherein the pathway comprises at least the enzymes AAE, OLS, OAC, and PT4; optionally, wherein the enzymes AAE, OLS, OAC, and PT4 have an amino acid sequence of at least 90% identity to SEQ ID NO: 2 (AAE), SEQ ID NO: 4 (OLS), SEQ ID NO: 6 (OAC), and SEQ ID NO: 8 or 10 (PT4), respectively.

20. The method of claim 10, wherein the pathway further comprises an enzyme capable of converting CBGA to Δ⁹-THCA, CBDA, and/or CBCA.

21. The method of claim 18, wherein the pathway comprises: (i) a CBDA synthase having an amino acid sequence of at least 90% identity to SEQ ID NO: 12 or 14; or (ii) a THCA synthase having an amino acid sequence of at least 90% identity to SEQ ID NO: 96 or 98.

22. A method for making a recombinant host cell for producing a cannabinoid comprising introducing into a host cell:
(a) a first set of nucleic acids that encode a pathway of enzymes capable of converting hexanoic acid to CBGA; and
(b) a heterologous nucleic acid that encodes a protein derived from a source that is not Cannabis sativa or Saccharomyces cerevisiae and is of a type selected from: a chaperonin, an efflux pump protein, a fatty acid pathway protein, a lipid transfer protein, a mevalonate pathway protein, a methylerythritol 4-phosphate pathway protein, a permease, and a terpenoid biosynthesis pathway protein.

23. (canceled)

24. (canceled)
